SEQUENCE LISTING (13 sequences)

SEQ ID NO: 1 - DNA, 1311 bp, Artificial Sequence
  Synthetic
atgtttgtgagggtgaaactgaacaagcccgtgaagtggtatagattctacagcacactggactcccactccctcaaactgcagagcggctccaagttcgtcaagatcaagcccgtgaacaatctgaggagctcctccagcgccgatttcgtgagccctcccaattccaagctccaatctctgatctggcagaatcccctccagaacgtgtacatcaccaagaagccttggacccccagcaccagagaagccatggtggagtttatcacccatctgcacgagagctatcccgaggtgaacgtcatcgtccagcccgacgtggctgaggagatcagccaagatttcaagagccccctcgaaaacgaccccaatagaccccatattctgtataccggccccgagcaagacatcgtcaataggaccgatctgctggtgacactgggaggagacggcaccattctgcatggcgtgtccatgtttggcaatacccaagtgcctcccgtgctggcctttgctctcggaacactgggctttctgctgcccttcgacttcaaggagcacaagaaggtgttccaagaggtgatcagcagcagagccaagtgcctccacagaacaagactggagtgccacctcaaaaagaaggacagcaactccagcatcgtgacccacgccatgaacgacatttttctgcatagaggcaatagcccccatctgaccaatctggacatcttcatcgatggcgaatttctgacaaggaccaccgctgacggcgtggctctggctacacctaccggctccaccgcctattctctgtccgccggcggatccattgtgagccctctggtccccgccattctgatgacccctatctgccctaggtctctgtcctttagacctctgattctgccccactcctcccacattagaatcaagatcggcagcaagctcaaccagaaacccgtgaactccgtggtcaagctgtccgtggacggcatcccccaacaagatctggacgtgggcgacgagatttacgtgatcaacgaggtgggcaccatctacatcgatggcacccaactgcccaccacaagaaaaaccgagaacgatttcaacaactccaagaagcctaagaggtccggcatttactgcgtggctaagacagagaacgactggatcagaggcatcaacgaactgctgggctttaacagctccttcagactgaccaagaggcagaccgacaacgatgattacaaggaccacgacggcgactacaaggatcacgacattgattataaggatgacgacgacaagtga

SEQ ID NO: 2 - DNA, 20 bp, Artificial Sequence
  Synthetic
gacaaagaagtcgcagacga

SEQ ID NO: 3 - DNA, 20 bp, Artificial Sequence
  Synthetic
ttgaggttcgtctagtaaag

SEQ ID NO: 4 - DNA, 20 bp, Artificial Sequence
  Synthetic
tgatgaagagactgttcgat

SEQ ID NO: 5 - DNA, 20 bp, Artificial Sequence
  Synthetic
gaagatgggcgggagtcttc

SEQ ID NO: 6 - DNA, 20 bp, Artificial Sequence
  Synthetic
cagacttaaacctgtcattg

SEQ ID NO: 7 - DNA, 20 bp, Artificial Sequence
  Synthetic
ctgctcgaactcgtagcggg

SEQ ID NO: 8 - DNA, 19 bp, Artificial Sequence
  Synthetic
ccagtgtggatgtcgacgt

SEQ ID NO: 9 - DNA, 20 bp, Artificial Sequence
  Synthetic
aggcaacaagatggcagctg

SEQ ID NO: 10 - DNA, 20 bp, Artificial Sequence
  Synthetic
tgcggcaggaggtagaagag

SEQ ID NO: 11 - DNA, 20 bp, Artificial Sequence
  Synthetic
tctgaacaacaagtactcgg

SEQ ID NO: 12 - DNA, 20 bp, Artificial Sequence
  Synthetic
aactccaggtctcatcgccg

SEQ ID NO: 13 - DNA, 20 bp, Artificial Sequence
  Synthetic
cagcagtaagcagccgcgtc

(19) United States
(12) Patent Application Publication
(10) Pub. No.: US 2024/0209353 A1
(43) Pub. Date: Jun. 27, 2024

(54) NADK2 INHIBITION IN CANCER AND FIBROTIC DISORDERS

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Craig B. Thompson, New York, NY (US); Simon Schwoerer, New York, NY (US); Jiajun Zhu, New York, NY (US)

(73) Assignees: Memorial Sloan-Kettering Cancer Center, New York, NY (US); Memorial Hospital for Cancer and Allied Diseases, New York, NY (US); Sloan-Kettering Institute for Cancer Research, New York, NY (US)

(21) Appl. No.: 18/285,925

(22) PCT Filed: Apr. 7, 2022
(86) PCT No.: PCT/US2022/023788

§ 371 Date: Oct. 6, 2023

Related U.S. Application Data
(60) Provisional application No. 63/172,598, filed on Apr. 8, 2021.

(51) Int. Cl. C12N 15/11; C12N 9/22

(52) U.S. Cl. CPC C12N 15/11; C12N 9/22; C12N 2310/20; C12N 2800/80

(57) ABSTRACT

Aspects of the disclosure provide methods for inhibiting cell proliferation and protein synthesis utilizing an antagonist of nicotinamide adenine dinucleotide kinase 2 (NADK2). In some aspects, these methods are used to treat a disease such as cancer or a disorder such as a fibrotic disorder. Further provided herein are compositions comprising a nutrient-deficient cell culture medium and an antagonist of NADK2.

### CROSS-REFERENCE TO RELATED APPLICATIONS

This application claims priority under 35 U.S.C. § 119(e) to U.S. Provisional Patent Application No. 63/172,598, filed Apr. 8, 2021, which is hereby incorporated by reference in its entirety.

### GOVERNMENT SUPPORT

This invention was made with government support under CA248711 and CA008748 awarded by the National Institutes of Health. The government has certain rights in this invention.

### REFERENCE TO A SEQUENCE LISTING SUBMITTED AS A TEXT FILE

The instant application contains a Sequence Listing which has been submitted in ASCII format and is hereby incorporated by reference in its entirety. Said ASCII copy, created on Apr. 6, 2022, is named S171570052WO00-SEQ-JIB and is 4510 bytes in size.

### BACKGROUND

Mammalian cells depend on the inter-conversion of nicotinamide adenine dinucleotide phosphate (NADP) molecules between the oxidized (NADP⁺) and reduced (NADPH) forms to support reductive biosynthesis and to maintain cellular antioxidant defense. NADP⁺ and NADPH molecules (also referred to as “NADP(H)”) are unable to cross subcellular membranes. As a result, cellular pools of NADP(H) are compartmentalized. In the cytosol, NADP(H) is derived from nicotinamide adenine dinucleotide [(NAD)H] by NAD kinase 1 (NADK1). Cytosolic NADPH acts as a substrate in fatty acid biosynthesis, and as the reducing equivalent required to regenerate reduced glutathione (GSH) and thioredoxin for antioxidant defense. Mitochondria host a number of biosynthetic activities critical for cellular metabolism but are also major sites for reactive oxygen species (ROS) generation. Mammalian mitochondrial NAD kinase 2 (NADK2) converts NAD(H) to NADP(H) through phosphorylation.

### SUMMARY

The present disclosure is based on the surprising discovery that mitochondrial nicotinamide adenine dinucleotide phosphate (NADPH) produced by nicotinamide adenine dinucleotide kinase 2 (NADK2) is critical to proline synthesis, protein synthesis, and maintaining cell proliferation in a nutrient-deficient environment. Inhibiting the activity of NADK2 inhibits protein synthesis and cell proliferation in vitro and in vivo. Thus, antagonists of NADK2 may be used to treat diseases or disorders characterized by increased protein synthesis (e.g., fibrosis) and/or increased cell proliferation (e.g., cancer).

In some aspects, the present disclosure provides a method of treating a cancer characterized as having an isocitrate dehydrogenase 2 (IDH2) mutation, the method comprising administering to a subject in need thereof an antagonist of nicotinamide adenine dinucleotide kinase 2 (NADK2) in an amount effective to treat the cancer.

In some aspects, the present disclosure provides a method for inhibiting cancer cell proliferation, the method comprising contacting cancer cells expressing a mutant IDH2 protein with an antagonist of NADK2, wherein the mutant IDH2 protein has neomorphic enzymatic activity.

In further aspects, the present disclosure provides a method for inhibiting cell proliferation comprising: providing a population of cells in a nutrient-deficient environment; and contacting a cell of the population of cells with an antagonist of NADK2, wherein the cell contacted with the antagonist has decreased proliferation compared to a cell not contacted with the antagonist of NADK2.

In further aspects, the present disclosure provides a composition comprising (i) a nutrient-deficient cell culture medium; and (ii) an antagonist of NADK2. In some embodiments, the nutrient-deficient cell culture medium is deficient in one or more amino acids. In some embodiments, the composition further comprises (iii) a population of cells. In some embodiments, the population of cells comprises cancer cells. In some embodiments, the cancer cells express a mutant IDH2 protein. In some embodiments, the nutrient-deficient cell culture medium comprises 10% serum, 100 units/mL penicillin, and/or 100 μg/mL streptomycin.

Accordingly, in some aspects, the present disclosure provides compositions and methods for use in treating a cancer and/or inhibiting proliferation of a cancer cell. In some embodiments, the cancer is characterized as having an isocitrate dehydrogenase 2 (IDH2) mutation. In some embodiments, the cancer is characterized as having increased levels of 2-hydroxyglutrarate (2HG) relative to a known reference value. In some embodiments, the cancer is characterized as having decreased levels of alpha-ketoglutarate (αKG) relative to a known reference value. In some embodiments, the known reference value is from a cell characterized as not having the IDH2 mutation. In some embodiments, the known reference value is from a non-cancerous cell and/or a cell that does not express a mutant IDH2 protein. In some embodiments, the cell is a non-cancerous cell of the subject. In some embodiments, the cancer is characterized as not having an isocitrate dehydrogenase 1 (IDH1) mutation.

In some embodiments, the IDH2 mutation produces a mutant IDH2 protein having a neomorphic enzymatic activity. In some embodiments, the neomorphic enzymatic activity is a reduction of αKG to 2HG. In some embodiments, the IDH2 mutation is selected from R172S, exon 4 mutation, a codon 140 missense mutation, R140Q, a codon 172 missense mutation, R172K, an amplification of IDH2, a loss of IDH2, R172W, R172M, R140W, R172G, V305M, H384Q, T350P. R172T, V355I, K155N, A416V, W21S, X39 splice, R159H, A347T, D390Y, D259N, A370T. A174T, or a combination thereof.

In some embodiments, the mutant IDH2 protein comprises one or more IDH2 mutations selected from R172S, exon 4 mutation, a codon 140 missense mutation, R140Q, a codon 172 missense mutation, R172K, an amplification of IDH2, a loss of IDH2, R172W, R172M, R140W, R172G, V305M, H384Q, T350P, R172T, V355I, K155N, A416V, W21S, X39 splice, R159H, A347T, D390Y, D259N, A370T, and A174T.

In some embodiments, the cancer is an adenocarcinoma. In some embodiments, the adenocarcinoma is selected from colon adenocarcinoma, lung adenocarcinoma, high grade ovarian serous adenocarcinoma, colorectal adenocarcinoma, rectal adenocarcinoma, prostate adenocarcinoma, or a combination thereof.

In some embodiments, the cancer is a carcinoma. In some embodiments, the carcinoma is selected from breast invasive ductal carcinoma, intrahepatic cholangiocarcinoma, endometrial endometrioid carcinoma, bladder urothelial carcinoma, endometrial carcinoma, squamous cell lung carcinoma, or a combination thereof.

In some embodiments, the cancer is selected from acute myeloid leukemia, oligodendroglioma, myelodysplastic syndrome, cutaneous melanoma, gliobastoma multiforme, angioimmunoblastic T-cell lymphoma, acute monoblastic and monocytic leukemia, or a combination thereof.

In further aspects, the present disclosure provides a method of treating a fibrotic disorder, the method comprising administering to a subject in need thereof an antagonist of NADK2 in an amount effective to treat the fibrotic disorder.

In some embodiments, the fibrotic disorder is characterized by increased levels of NADK2 relative to a known reference value. In some embodiments, the fibrotic disorder is characterized by increased levels of pyrroline-5-carboxylate synthase (P5CS) relative to a known reference value. In some embodiments, the known reference value is from a normal cell of the subject.

In some embodiments, the fibrotic disorder is characterized by increased levels of an extracellular matrix protein. In some embodiments, the extracellular matrix protein is collagen, elastin, fibronectin, and/or laminin. In some embodiments, the fibrotic disorder is pulmonary fibrosis or liver fibrosis.

In further aspects, the present disclosure provides a method for inhibiting protein synthesis, the method comprising contacting a cell from a population of cells with an antagonist of NADK2.

In some embodiments, the protein synthesis is decreased as compared to a cell that has not been contacted with the NADK2 antagonist. In some embodiments, the cell that has not been contacted with the antagonist is from the population of cells.

In some embodiments, the cell from the population of cells is contacted with the antagonist in a nutrient-deficient environment. In some embodiments, the nutrient-deficient environment has reduced levels of one or more amino acids compared to a nutrient-replete environment. In some embodiments, the nutrient-deficient environment contains a maximum of 300 μM of proline.

In some embodiments, the cytosolic protein is collagen, elastin, fibronectin, and/or laminin. In some embodiments, the cytosolic protein is collagen, and collagen synthesis is decreased in the cell contacted with the NADK2 antagonist as measured by staining collagen protein. In some embodiments, the collagen protein is stained by Picrosirius red staining.

In some embodiments, proline biosynthesis is decreased in the cell contacted with the NADK2 antagonist as measured by gas chromatography-mass spectrometry (GC-MS) and/or liquid chromatography-mass spectrometry (LC-MS). In some embodiments, proline is labeled with an isotopologue.

In some aspects, the present disclosure provides a method for decreasing protein synthesis, the method comprising: providing a cell expressing nicotinamide adenine dinucleotide kinase 2 (NADK2) in a nutrient-deficient environment; and contacting the cell with an antagonist of NADK2, wherein the cell contacted with the antagonist has decreased protein synthesis compared to a control cell not contacted with the antagonist.

In some embodiments, the protein (e.g., the protein having decreased synthesis) is collagen, elastin, fibronectin, and/or laminin. Accordingly, in some embodiments, the method is a method for decreasing synthesis of collagen, elastin, fibronectin, and/or laminin.

In some embodiments, the nutrient-deficient environment is deficient in one or more amino acids. In some embodiments, the nutrient-deficient environment is in vitro. In some embodiments, the nutrient-deficient environment is in vivo. In some embodiments, the nutrient-deficient environment comprises a subject on a restrictive diet.

In some embodiments, the cell contacted with the antagonist has reduced survival and/or proliferation compared to the control cell not contacted with the antagonist. In some embodiments, the cell contacted with the antagonist expresses pyrroline-5-carboxylate synthase (P5CS). In some embodiments, the cell contacted with the antagonist is associated with a fibrotic disorder. In some embodiments, the cell contacted with the antagonist expresses increased levels of NADK2 compared to a cell not associated with a fibrotic disorder. In some embodiments, the fibrotic disorder is pulmonary fibrosis or liver fibrosis. In some embodiments, the cell contacted with the antagonist expresses increased levels of P5CS compared to a cell not associated with a fibrotic disorder.

The details of certain embodiments of the invention are set forth in the Detailed Description, as described below. Other features, objects, and advantages of the invention will be apparent from the Examples, Drawings, and Claims.

### BRIEF DESCRIPTION OF THE DRAWINGS

The accompanying drawings, which constitute a part of this specification, illustrate several embodiments of the invention and together with the description, serve to explain the principles of the invention.

FIGS. 1A-1G show that NAKD2 is required to maintain the mitochondrial NADP(H) pool. FIG. 1A shows DLD1 cells expressing hemagglutinin-tagged (HA-tagged) OMP25 protein (DLD1-OMP25HA) engineered to express control guide RNA (sgCtrl) or two independent guide RNA sequences targeting NADK2 (sgNADK2-1 and sgNADK2-2), and subjected to Western blot of whole cell or anti-HA immunopurified mitochondria (Mito-IP). FIGS. 1B-1C show colorimetric enzyme-based measurement of total NADP(H) abundance in whole cell (FIG. 1B) or immunopurified mitochondria (FIG. 1C) of DLD1-OMP25HA cells treated with sgCtrl, sgNADK2-1, or sgNADK2-2, cultured in Dulbecco's Modified Eagle Medium/F12 medium (DMEM/F12 medium). FIG. 1D shows Western blot analysis of JJ012 cells expressing mutant isocitrate dehydrogenase 1 (IDH1) and CS1 cells expressing mutant isocitrate dehydrogenase 2 (IDH2) treated with sgCtrl, sgNADK2-1, or sgNADK2-2. FIGS. 1E-1F show 2-hydroxyglutarate (2HG) abundance measured by gas chromatography-mass spectrometry (GC-MS) in JJ012 (FIG. 1E) and CS1 (FIG. 1F) cells treated with sgCtrl, sgNADK2-1, or sgNADK2-2. FIG. 1G shows 2HG abundance measured by GC-MS in xenograft tumors formed by CS1 cells treated with sgCtrl or sgNADK2-2. The error bars in FIG. 1B represent mean+SD, n=6. The error bars in FIGS. 1C, 1E, and 1F represent mean+SD, n=3. The error bars in FIG. 1G represent mean+SD, n=10. In FIG. 1C, a one-way ANOVA was performed with matched measures. In FIG. 1F, a one-way ANOVA was performed. In FIG. 1G, a two-sided t-test was performed with Welch's correction. *** P<0.001.

FIGS. 2A-2L show that mitochondrial NADP(H) depletion does not significantly affect the folate pathway, tricarboxylic acid cycle (TCA cycle) activity, or measures of oxidative stress. FIG. 2A shows a scheme of the tracing strategy. Catabolismof [2.3.3-²H3]serine in the mitochondrial or cytosolic folate pathway produces singly or doubly deuterated thymidine triphosphate (TTP M+1 or TTP M+2), respectively. FIG. 2B shows a Western blot of DLD1 cells treated with sgCtrl, sgNADK2-1, sgNADK2-2, sgMTHFD2, or sgSHMT2. FIG. 2C shows isotopologue distribution of TTP measured by liquid chromatography-mass spectrometry (LC-MS) in DLD1 cells denoted in FIG. 2B cultured in [2.3.3-2H3]serine-containing medium for 8 hours. FIGS. 2D-2G show isotopologue distribution of citrate (FIG. 2D), alpha-ketoglutarate (αKG, FIG. 2E), fumarate (FIG. 2F), and malate (FIG. 2G) measured by GC-MS in DLD1 cells treated with sgCtrl, sgNADK2-1, or sgNADK2-2, cultured in [U-¹³C]glutamine-containing medium for 6 hours. FIG. 2H shows cellular reactive oxygen species (ROS) measured by CM-H₂DCFDA (5-(and-6)-chloromethyl-2′,7′-dichlorodihydrofluorescein diacetate, acetyl ester) in the indicated DLD1 cells, mock treated or treated with 150 μM H₂O₂ for 4 hours. FIG. 2I shows DLD1 cells expressing Mito-Orp1-roGFP2 and the indicated sgRNA were treated with vehicle (DMSO) or 100 μM MitoParaquat (MitoPQ) for 24 hours. Oxidation status was expressed as percentage of maximal oxidation which was determined by treating cells with 5 mM H₂O₂ for 5 min before harvest. FIG. 2J shows Western blot analysis of whole cell or immunopurified mitochondria of DLD1-OMP25HA cells expressing the indicated sgRNA. FIG. 2K shows Western blot of the indicated DLD1 cells mock treated or treated with 500 μM H₂O₂ for 6 hours. “SE” means short exposure and “LE” means long exposure. FIG. 2L shows ferroptosis sensitivity of the indicated DLD1 cells, measured as percentage cell death upon mock. Erastin (5 μM) or RSL3 (0.5 μM) treatment for 24 hours. All error bars represent mean+SD, n=3.

FIGS. 3A-3I show that mitochondrial NADP(H) depletion results in proline auxotrophy. FIGS. 3A-3B show cell proliferation measured as cell number fold change (Day 4/Day 0) of T47D cells treated with sgCtrl, sgNADK2-1, or sgNADK2-2, cultured in the indicated medium and supplementation. “LA” is lipoic acid. “Pyr” is pyruvate. “Cu” is cupric sulfate. “Zn” is zinc sulfate. “B12” is vitamin B12. “A” is alanine. “D” is aspartate. “N” is asparagine. “E” is glutamate, and “P” is proline. All supplements were added at the concentrations present in DMEM/F12. FIG. 3C shows proline abundance measured by GC-MS in the indicated T47D cells cultured in DMEM. FIGS. 3D-3F show a Western blot (FIG. 3D), proline abundance measured by GC-MS (FIG. 3E), and cell proliferation (FIG. 3F) of DMEM-cultured T47D cells treated with sgCtrl or sgNADK2-2 and ectopically expressing vector or NADK2 cDNA resistant to sgNADK2-2 mediated CRISPR-Cas9 genome editing. FIGS. 3G-3I show a Western blot (FIG. 3G), proline abundance measured by GC-MS (FIG. 3H), and cell proliferation (FIG. 3I) of DMEM-cultured T47D cells treated with sgCtrl, sgNADK2-1, or sgNADK2-2 and ectopically expressing vector or the POS5 cDNA. All error bars represent mean+SD, n=3. In FIGS. 3A-3C, 3H, and 3I, one-way ANOVA was performed. In FIGS. 3E and 3F, a two-sided t-test was performed with Welch's correction. ** P<0.01; *** P<0.001; n.s., P>0.05.

FIGS. 4A-4O show that the mitochondrial NADP(H) pool is required to support proline biosynthesis and collagen production. FIG. 4A shows a heatmap representing changes of metabolite levels measured by GC-MS in T47D cells treated with sgCtrl, sgNADK2-1, or sgNADK2-2 cultured in DMEM for 48 hours. The average of 3 biological replicates is shown. For each metabolite, values of sgNADK2-1 and sgNADK2-2 cells are shown as log 2 (fold change) relative to the value of sgCtrl cells. FIG. 4B shows changes of metabolite levels measured by GC-MS in DMEM/F12 medium used to culture T47D cells treated with sgCtrl, sgNADK2-1, or sgNADK2-2 for 48 hours. FIGS. 4C-4D show the proline (FIG. 4C) and glutamate (FIG. 4D) data from FIG. 4B re-plotted as normalized values to sgCtrl cells. FIG. 4E shows proline abundance measured by GC-MS in xenograft tumors formed by CS1 cells with sgCtrl or sgNADK2-2. FIG. 4F shows a scheme of proline biosynthesis pathway in the mitochondria. FIGS. 4G-4J shows relative total level and isotopologue distribution of glutamate (FIG. 4G), proline (FIG. 4H), ornithine (FIG. 4I), and putrescine (FIG. 4J) measured by LC-MS in mouse embryonic fibroblast cells (MEFs) treated with sgCtrl, sgNADK2-1, or sgNADK2-2 and cultured in DMEM containing [U-¹³C]glutamine for 8 hours. FIG. 4K shows a Western blot of the indicated MEFs, cultured in DMEM or DMEM supplemented with 300 UM proline. FIG. 4L shows a scheme of extracellular matrix (ECM) extraction and collagen staining in cells and under conditions described in FIG. 4M. FIG. 4M shows secreted collagen levels quantified by Picro sirius red staining in ECM derived from MEFs treated with sgCtrl, sgNADK2-1, or sgNADK2-2, cultured for 48 hours in DMEM or DMEM supplemented with 300 μM proline, in the presence of 50 μM ascorbate. FIG. 4N shows a Pearson correlation of NADK2 mRNA level and forced vital capacity (FVC) before bronchodilator (pre-BD) as percentage of what was predicted for each patient. Data from the GSE32537 accession data set. FIG. 4O shows a Pearson correlation of NADK2 mRNA level and diffusing capacity for carbon monoxide (DLCO) as a percentage of what was predicted for each patient. Data from GSE32537. Error bars in FIG. 4E represent mean+SD, n=10. All other error bars represent mean+SD, n=3. In FIGS. 4B-4D, one-way ANOVA was performed. In FIG. 4E and FIG. 4M, a two-sided t-test was performed with Welch's correction. * P<0.05; ** P<0.01; *** P<0.001.

FIGS. 5A-5O show that NAKD2 is required to maintain the mitochondrial NADP(H) pool. FIGS. 5A-5C show Western blot analysis of subcellular fractionation samples from DLD1 cells (FIG. 5A), 293T cells (FIG. 5B), and U2OS cells (FIG. 5C). FIG. 5D shows Western blot analysis in DLD1 cells. FIG. 5E shows Western blot analysis of whole cell or anti-HA immunopurified mitochondria (Mito-IP) of DLD1 cells expressing HA-tagged OMP25 or the Myc-tagged OMP25 as control. FIGS. 5F-5G show peak areas of ribose-5-phosphate, dihydroxyacetone phosphate (DHAP), glucosamine, alpha-ketoglutarate (αKG), succinate, and malate as measured by LC-MS in whole cell (FIG. 5F) or mitochondrial immunoprecipitation (Mito-IP) (FIG. 5G) samples of DLD1-OMP25HA cells treated with sgCtrl, sgNADK2-1, or sgNADK2-2. Ribose-5-phosphate. DHAP, and glucosamine are known to be excluded from the mitochondrial compartment. A full list of all detected metabolites was annotated and included in Tables 1A-1G. FIGS. 5H-5J show colorimetric enzyme-based measurement of total NADP(H) abundance in whole cell (FIG. 5H), NADP+ to NADPH ratio in whole cell (FIG. 5I), and total NADP(H) abundance in immunopurified mitochondria (FIG. 5J) of DLD1 cells treated with sgCtrl, sgNADK2-1, or sgNADK2-2, cultured in DMEM medium. FIGS. 5K-5N show colorimetric enzyme-based measurement of total NAD(H) abundance in whole cell (FIG. 5K). NAD+ to NADH ratio in whole cell (FIG. 5L), total NAD(H) abundance in immunopurified mitochondria (FIG. 5M), and NAD+ to NADH ratio in immunopurified mitochondria (FIG. 5N) of DLD1 cells treated with sgCtrl, sgNADK2-1, or sgNADK2-2, cultured in DMEM/F12 medium. The NAD phosphoribosyltransferase (NAMPT) inhibitor FK866 is used at 50 nM for 24 hours in FIG. 5K. FIG. 5O shows a scheme of NADPH-dependent 2HG production by mutant IDH1 and mutant IDH2, in cytosol and in mitochondria, respectively. Error bars represent mean+SD, n=3. In FIG. 5J, one-way ANOVA was performed with matched measures. * P<0.05.

FIGS. 6A-6Y show that mitochondrial NADP(H) depletion does not significantly affect the folate pathway. TCA cycle activity, or measures of oxidative stress. FIG. 6A shows Western blot analysis of HaCaT cells treated with sgCtrl, sgNADK2-1, sgNADK2-2, sgMTHFD2, or sgSHMT2. FIG. 6B shows isotopologue distribution of thymidine triphosphate (TTP) measured by LC-MS in HaCaT cells denoted in FIG. 6A, cultured in [2.3.3-2H3]serine-containing medium for 8 hours. FIGS. 6C-6F shows isotopologue distribution of citrate (FIG. 6C), alpha-ketoglutarate (αKG) (FIG. 6D), fumarate (FIG. 6E), and malate (FIG. 6F) measured by GC-MS in DLD1 cells cultured in [U-¹³C]glucose-containing medium for 6 hours. FIGS. 6G-6J show citrate (FIG. 6G), αKG (FIG. 6H), fumarate (FIG. 6I), and malate (FIG. 6J) measured by GC-MS in HaCaT cells cultured in [U-¹³C]glutamine-containing medium for 6 hours. FIGS. 6K-6N show citrate (FIG. 6K), αKG (FIG. 6L), fumarate (FIG. 6M), and malate (FIG. 6N) measured by GC-MS in HaCaT cells cultured in [U-¹³C]glucose-containing medium for 6 hours. FIGS. 6O-6R show citrate (FIG. 6O), αKG (FIG. 6P), fumarate (FIG. 6Q), and malate (FIG. 6R) measured by GC-MS in MEF cells cultured in [U-¹³C]glutamine-containing medium for 6 hours. FIGS. 6S-6V show citrate (FIG. 6S), αKG (FIG. 6T), fumarate (FIG. 6U), and malate (FIG. 6V) measured by GC-MS in MEF cells cultured in [U-¹³C]glucose-containing medium for 6 hours. FIGS. 6W-6Y show oxygen consumption rate (OCR) measured using the Seahorse bioanalyzer in DLD1 cells (FIG. 6W). HaCaT cells (FIG. 6X), and MEFs (FIG. 6Y) cultured in DMEM/F12 media. “Oligo” is oligomycin. “Rot/Anti-A” is rotenone/antimycin, and “PCV” is packed cell volume. Error bars in FIGS. 6B-6V represent mean+SD, n=3. Error bars in FIGS. 6W-6Y represent mean+SD, n=8.

FIGS. 7A-7W show that mitochondrial NADP(H) depletion does not significantly affect the folate pathway. TCA cycle activity, or measures of oxidative stress. FIGS. 7A-7C show cellular reactive oxygen species (ROS) measured by CM-H2DCFDA (5-(and-6)-chloromethyl-2′,7′-dichlorodihydrofluorescein diacetate, acetyl ester) in DLD1 cells (FIG. 7A). T47D cells (FIG. 7B), and HaCaT cells (FIG. 7C) treated with sgCtrl, sgNADK2-1, or sgNADK2-2. FIG. 7D shows cellular ROS measured by CM-H2DCFDA in the indicated T47D cells that were mock treated or treated with 200 μM H₂O₂ for 4 hours. FIGS. 7E-7G show mitochondrial superoxide measured by mitochondrial superoxide (MitoSox) in DLD1 cells (FIG. 7E), T47D cells (FIG. 7F), and HaCaT cells (FIG. 7G) with sgCtrl, sgNADK2-1, or sgNADK2-2, mock treated or treated with Rotenone (0.5 μM) for 4 hours. FIG. 7H shows HaCaT cells with sgCtrl, sgNADK2-1, or sgNADK2-2 engineered to express Mito-Orp1-roGFP2 and treated with vehicle (DMSO) or 100 μM MitoPQ for 24 hours. Oxidation status was expressed as percentage of maximal oxidation which was determined by treating cells with 5 mM H₂O₂ for 5 min before harvest. FIGS. 7I-7J shows DLD1 cells (FIG. 7I) and T47D cells (FIG. 7J) treated with sgCtrl, sgNADK2-1, or sgNADK2-2 engineered to express Mito-Grx1-roGFP2 and mock treated or treated with 100 μM H₂O₂ for 4 hours. Oxidation status was expressed as percentage of maximal oxidation which was determined by treating cells with 5 mM H₂O₂ for 5 min before harvest. FIGS. 7K-7L show Western blot analysis of the indicated DLD1 cells (FIG. 7K) and T47D cells (FIG. 7L) treated with vehicle (DMSO) or 100 UM MitoPQ for 24 hours. FIGS. 7M-7P show the results of a luminescent-based GSH/GSSG-Glo assay of total GSH abundance in whole cell (FIG. 7M), GSH to GSSG ratio in whole cell (FIG. 7N), total GSH abundance in immunopurified mitochondria (FIG. 7O), and GSH to GSSG ratio in immunopurified mitochondria (FIG. 7P) of DLD1-OMP25HA cells treated with sgCtrl, sgNADK2-1, or sgNADK2-2. “BSO” is buthionine sulfoximine, used at 100 μM for 24 hours in FIG. 7M. FIGS. 7Q-7R show isotopologue distribution of GSH (FIG. 7Q) and GSSG (FIG. 7R) measured by LC-MS of the indicated T47D cells, cultured in [U-¹³C]glutamine-containing medium for 8 hours. These results in FIGS. 7Q-7R are from the same experiment as FIGS. 12A-12B. FIGS. 7S-7T show Western blot analysis of T47D cells (FIG. 7S) and HaCaT cells (FIG. 7T) treated with sgCtrl, sgNADK2-1, or sgNADK2-2 that were mock treated or treated with 100 μM H₂O₂ (FIG. 7S) and 500 μM H₂O₂ (FIG. 7T) for 6 hours. “SE” is short exposure and “LE” is long exposure. FIG. 7U shows ferroptosis sensitivity of T47D cells treated with sgCtrl, sgNADK2-1, or sgNADK2-2, measured as percentage cell death upon mock. Erastin (10 μM) or RSL3 (5 μM) treatment for 48 hours. FIG. 7V shows Western blot analysis of proliferative MEFs or contact-inhibited MEFs treated with sgCtrl, sgNADK2-1, or sgNADK2-2. FIG. 7W shows ferroptosis sensitivity of contact-inhibited MEFs treated with sgCtrl, sgNADK2-1, or sgNADK2-2, measured as percentage cell death upon mock or Erastin (10 μM) treatment for 24 hours. Error bars in FIG. 7W represent mean+SD, n=4. All other error bars represent mean+SD, n=3.

FIGS. 8A-8M show that mitochondrial NADP(H) depletion results in proline auxotrophy. FIG. 8A shows Western blot analysis in T47D cells. FIG. 8B shows cell proliferation measured as cell number fold change (Day 4/Day 0) of T47D cells treated with sgCtrl, sgNADK2-1, or sgNADK2-2, cultured in DMEM or DMEM/F12 based medium. FIG. 8C shows Western blot analysis in MCF10A cells. FIG. 8D shows cell proliferation measured as cell number fold change (Day 2/Day 0) of MCF10A cells treated with sgCtrl, sgNADK2-1, or sgNADK2-2, cultured in DMEM or DMEM/F12 based medium. FIGS. 8D-8H show cell proliferation measured as cell number fold change of the indicated cells with sgCtrl, sgNADK2-1, or sgNADK2-2, cultured in the indicated medium and supplementation. “LA” is lipoic acid, “Pyr” is pyruvate. “Cu” is cupric sulfate. “Zn” is zinc sulfate, “B12” is vitamin B12. “A” is alanine. “D” is aspartate. “N” is asparagine. “E” is glutamate, and “P” is proline. All the supplements were added at the concentrations present in the DMEM/F12 medium. FIG. 8I shows Western blot analysis in HaCaT cells. FIG. 8J shows cell proliferation measured as cell number fold change (Day 2/Day 0) of HaCaT cells with sgCtrl, sgNADK2-1, orsgNADK2-2, cultured in DMEM or DMEM supplemented with 150 μM proline. FIG. 8K shows proline abundance measured by GC-MS in DLD1 cells with sgCtrl, sgNADK2-1, or sgNADK2-2, cultured under normoxia (20% O₂) or hypoxia (0.5% O₂) for 48 hours. FIG. 8L shows cell proliferation measured as cell number fold change (Day 3/Day 0) of DLD1 cells treated with sgCtrl, sgNADK2-1, or sgNADK2-2, cultured in DMEM or DMEM supplemented with 150 μM proline. FIG. 8M shows cell proliferation measured as cell number fold change (Day 4/Day 0) of T47D cells treated with sgCtrl, sgNADK2-1, or sgNADK2-2, cultured in DMEM/F12 or proline-deficient DMEM/F12. All error bars represent mean+SD, n=3. In FIGS. 8B, 8D-8H, and 8J-8M, one-way ANOVA was performed. *** P<0.001.

FIGS. 9A-9F show that mitochondrial NADP(H) depletion results in proline auxotrophy. FIGS. 9A-9C show Western blot analysis (FIG. 9A), proline abundance measured by GC-MS (FIG. 9B), and cell proliferation (FIG. 9C) of DMEM-cultured T47D cells treated with sgCtrl or sgNADK2-1 and ectopically expressing vector or NADK2 cDNA resistant to sgNADK2-1 mediated CRISPR-Cas9 genome editing. FIGS. 9D-9F show Western blot analysis (FIG. 9D), proline abundance measured by GC-MS (FIG. 9E), and cell proliferation (FIG. 9F) of DMEM-cultured MEFs treated with sgCtrl, sgNADK2-1, or sgNADK2-2 and ectopically expressing vector or the POS5 cDNA. All error bars represent mean+SD, n=3. In FIGS. 9E-9F, one-way ANOVA was performed. In FIGS. 9B-9C, a two-sided t-test was performed with Welch's correction. * P<0.05; *** P<0.001.

FIGS. 10A-10J show that the mitochondrial NADP(H) pool is required to support proline biosynthesis and collagen production. FIGS. 10A-10B show heatmaps representing changes of metabolite measured by GC-MS in DLD1 (FIG. 10A) and HaCaT cells (FIG. 10B) treated with sgCtrl, sgNADK2-1, or sgNADK2-2 and cultured in DMEM for 48 hours. The average of 3 biological replicates was shown. For each metabolite, values of sgNADK2-1 and sgNADK2-2 cells are shown as log 2 (fold change) relative to the value of sgCtrl cells. FIGS. 10C-10D show proline abundance measured by GC-MS in proliferative (FIG. 10C) and contact-inhibited MEFs (FIG. 10D) treated with sgCtrl, sgNADK2-1, or sgNADK2-2. FIG. 10E shows Western blot analysis in HaCaT cells. FIG. 10F shows proline abundance measured by GC-MS in HaCaT cells treated with sgCtrl, sgNADK1-1, or sgNADK1-2. FIG. 10G shows Western blot analysis in U2OS cells ectopically expressing GFP control, or FLAG-tagged cytosol oxygen-dependent NADPH oxidase (cytoTPNOX) or mitochondrial oxygen-dependent NADPH oxidase (mitoTPNOX). FIG. 10H shows a heatmap representing changes of metabolite measured by GC-MS in U2OS cells denoted in FIG. 10G. The average of 3 biological replicates is shown. For each metabolite, values of cytoTPNOX- and mitoTPNOX-expressing cells were shown as log 2 (fold change) relative to the value of GFP-expressing cells. FIG. 10I shows Western blot analysis in MEFs ectopically expressing control vector, or FLAG-tagged cytoTPNOX or mitoTPNOX. FIG. 10J shows a heatmap representing changes of metabolite measured by GC-MS in MEFs denoted in FIG. 10I. The average of 3 biological replicates was shown. For each metabolite, values of cytoTPNOX- and mitoTPNOX-expressing cells were shown as log 2 (fold change) relative to the value of control vector-expressing cells. All error bars represent mean+SD, n=3. In FIGS. 10C-10D, one-way ANOVA was performed. *** P<0.001.

FIGS. 11A-11I show that the mitochondrial NADP(H) pool is required to support proline biosynthesis and collagen production. FIGS. 11A-11B show changes of metabolite measured by GC-MS in DMEM/F12 medium used to culture DLD1 cells (FIG. 11A) and HaCaT cells (FIG. 11B) with sgCtrl, sgNADK2-1, or sgNADK2-2 for 48 hours. In FIGS. 11C-11F, proline levels in DLD1 cells (FIG. 11C), proline levels in HaCaT cells (FIG. 11D), glutamate levels in DLD1 cells (FIG. 11E), and glutamate levels in HaCaT cells (FIG. 11F) (data from FIGS. 11A-11B and re-plotted as normalized values to the corresponding sgCtrl cells). FIG. 11G shows abundance of the indicated amino acids measured by GC-MS in xenograft tumors formed by CS1 cells treated with sgCtrl or sgNADK2-2. FIG. 11H shows growth of xenograft tumors formed by CS1 cells treated with sgCtrl or sgNADK2-2. FIG. 11I shows abundance of the indicated amino acids measured by GC-MS in the plasma of tumor-xenografted mice, assayed at the time of tumor resection. Error bars in FIGS. 11A-11F represent mean+SD, n=3. Error bars in FIG. 11G represent mean+SD, n=10. Error bars in FIG. 11H represent mean±SEM, n=10. Error bars in FIG. 11I represent mean+SD, n=5. In FIGS. 11A-11F, one-way ANOVA was performed. In FIG. 11G, a two-sided t-test was performed and adjusted for multiple comparisons using the Holm-Sidak method. In FIG. 11H, two-way ANOVA was performed with matched measures. ** P<0.01; *** P<0.001.

FIGS. 12A-12K show that the mitochondrial NADP(H) pool is required to support proline biosynthesis and collagen production. FIGS. 12A-12B show relative total level and isotopologue distribution of the indicated metabolites in T47D cells with sgCtrl, sgNADK2-1, or sgNADK2-2, cultured in DMEM containing [U-¹³C]glutamine for 8 hours. FIG. 12C shows Western blot analysis of T47D cells. FIG. 12D shows proline abundance measured by GC-MS in T47D cells with sgCtrl, sgPYCRL-1, or sgPYCRL-2 cultured in DMEM for 48 hours. FIG. 12E shows a scheme of potential metabolites traced by [U-¹³C]glutamine (filled circles) and [U-¹³C]arginine (open circles). FIGS. 12F-12K show percentage of ornithine labeled with [U-¹³C]glutamine (FIG. 12F), putrescine labeled with [U-¹³C]glutamine (FIG. 12G), ornithine labeled with [U-¹³C]arginine (FIG. 12H), putrescine labeled with [U-¹³C]arginine (FIG. 12I), citrulline labeled with [U-¹³C]glutamine (FIG. 12J), and citrulline labeled with [U-¹³C]arginine (FIG. 12K) isotopologues in MEFs with sgCtrl, sgNADK2-1, or sgNADK2-2. FIGS. 12F-12G are data from FIGS. 4I-4J replotted as percentages of isotopologue distributions. All cells were cultured in DMEM containing the corresponding [U-¹³C]-labeled reagents for 8 hours before the metabolite measurement. All error bars in this Figure represent mean+SD, n=3.

FIGS. 13A-13F show that the mitochondrial NADP(H) pool is required to support proline biosynthesis and collagen production. FIG. 13A shows Western blot analysis of NIH-3T3 cells cultured in DMEM or DMEM supplemented with 300 μM proline. FIG. 13B shows Western blot analysis of MEFs cultured in DMEM or DMEM supplemented with the indicated amino acids. “A” is alanine. “D” is aspartate. “N” is asparagine. “E” is glutamate, and “P” is proline. All amino acid supplements were added at the concentrations present in DMEM/F12 medium. FIGS. 13C-13D show Saos2 cells (FIG. 13C) and CS1 cells (FIG. 13D) cultured in DMEM or DMEM supplemented with 300 μM proline. FIGS. 13E-13F show idiopathic pulmonary fibrosis (IPF) patients from the GSE32537 accession data set were assigned into NADK2^{low}/P5CS^{low} and NADK2^{high}/P5CS^{high} groups based on the expression level of NADK2 and P5CS. “High” represents patients with NADK2 or P5CS expression values being above the 75% percentile of the respective gene expression; “low” represents patients with expression values being below the 25% percentile of gene expression. FIG. 13E shows the forced vital capacity (FVC) before bronchodilator (pre-BD) as percentage of what was predicted for each patient, and FIG. 13F shows the diffusing capacity for carbon monoxide (DLCO) as percentage of what was predicted for each patient were compared between the groups. In FIGS. 13E-13F, a two-sided t-test was performed with Welch's correction, the number of samples in each group was indicated on the plot.

FIGS. 14A-14H show that proline biosynthesis is required for collagen production by fibroblasts in vitro. FIG. 14A shows a Western blot of NIH-3T3 cells expressing sgCtrl or sgP5CS-2 and treated with TGFβ or mock for 48 hours in the presence or absence of 0.15 mM proline. FIG. 14B shows collagen abundance in extracellular matrix (ECM) produced by NIH-3T3 cells expressing sgCtrl or sgP5CS-2 grown in the presence of absence of TGFβ and 0.15 mM proline, measured by Picrosirius red staining, and normalized to the packed cell volume of cells grown on a parallel plate under identical conditions. Values are relative to mock-treated sgCtrl-expressing cells. FIG. 14C shows proline abundance in NIH-3T3 cells expressing empty vector or HA-P5CS cDNA, measured by gas chromatography-mass spectrometry (GC-MS). Values are relative to mock-treated empty vector-expressing cells. FIG. 14D shows a Western blot of NIH-3T3 cells expressing empty vector or HA-P5CS cDNA. FIG. 14E shows collagen abundance in ECM produced by NIH-3T3 cells expressing empty vector or HA-P5CS cDNA, measured by Picrosirius red staining, and normalized to the packed cell volume of cells grown on a parallel plate under identical conditions. Values are relative to mock-treated empty vector-expressing cells. P<0.0001 (sgP5CS±proline in mock and TGFβ-treated cells). FIGS. 14F-14G show analysis of the indicated gene expression datasets for mRNA levels of P5CS. FIG. 14F show lung tissue from mice with pulmonary fibrosis induced by bleomycin (Bleo) treatment compared to saline treatment (GSE112827). FIG. 14G shows two datasets (GSE110147, GSE32537) from lungs of patients with idiopathic pulmonary fibrosis (IPF) compared to normal controls (Ctrl). AU, arbitrary units. The number of patients per group is indicated. FIG. 14H shows Pearson's correlation of P5CS mRNA level and forced vital capacity (FVC) before bronchodilator (pre-BD) as percentage of what was predicted for each patient, from clinical data of GSE32537. P-values were calculated by two-sided unpaired t-test with Welch's correction (FIGS. 14C, 14E), by two-way ANOVA with Holm-Sidak multiple comparison test (FIG. 14B), by moderated t-statistics and adjustment for multiple comparisons with the Benjamini and Hochberg false discovery rate method (FIGS. 14F-14G), or by Pearson's correlation (FIG. 14H). Bars in FIGS. 14B, 14C, and 14E represent the mean+SD; lines in FIG. 14F represent the mean±SD; data in FIG. 14G represent median with 50% confidence interval box and 95% confidence interval whiskers; and line in FIG. 14H represents linear regression with the SD shown as dotted lines, n=3 (FIGS. 14B, 14C, and 14E); n=3 (saline), n=5 (bleomycin) FIG. 14F; n=11 (Ctrl, left), n=22 (IPF, left); n=50 (Ctrl, right), n=119 (IPF, right) FIG. 14G; n=117 FIG. 14H. A representative experiment is shown in (FIGS. 14A, 14D).

FIGS. 15A-15H show that fibroblast pyruvate carboxylase (PC) supports pancreatic and mammary tumor growth and fibrosis. FIG. 15A shows a growth curve of pancreatic ductal adenocarcinoma (KPC) and KPC/pancreatic stellate cells (PSCs) allograft tumors. FIG. 15B shows representative images of Masson's Trichome staining of KPC/PSC allograft tumors. Scale bar=500 μm. FIG. 15C shows a quantification of Masson's Trichome staining of KPC/PSC allograft tumors as a percent of total tumor area, n=8. FIG. 15D shows hydroxyproline concentration in acid hydrosylates of mouse mammary tumor (DB7) and primary mammary fibroblasts (MFB) DB7/MFB allograft tumors harvested 8 days after injection. FIG. 15E shows a Western blot of lysates from DB7 and DB7/MFB allograft tumors harvested 8 days after injection. FIG. 15F shows quantification of collagen I band intensity relative to Actin from Western blots in FIG. 15E, n=6 (DB7 alone), n=7 (DB7+MFB Ctrl, DB7+MFB PC-knockout (PC-ko)). FIG. 15G shows representative images of Picrosirius staining of KPC/PSC allograft tumors. Scale bar=500 μm. FIG. 15H shows quantification of Picrosirius staining of KPC/PSC allograft tumors as percent of total tumor area, n=8. Data represent mean±SEM (FIG. 15A), median with 25% to 75% percentile box and min/max whiskers (FIGS. 15C, 15D, 15F). P-values were calculated by two-way ANOVA (FIG. 15A) analyzing the effects of PC-ko or GluI-ko on spheroid or tumor growth over time, by one-way ANOVA (FIG. 15C), by one-way ANOVA with Holm-Sidak correction for multiple comparisons (FIGS. 15D, 15F), or by one-way ANOVA (FIGS. 15G-15H).

### DETAILED DESCRIPTION

Aspects of the present disclosure relate to the discovery that NADPH produced by NADK2 is required for proline biosynthesis, cytosolic protein synthesis, and cell proliferation in a nutrient-deficient environment. Cells contacted with an antagonist of NADK2 in a nutrient-deficient environment will have reduced proliferation due to decreased proline biosynthesis. Thus, methods and compositions provided herein may be used to treat disorders (e.g., cancer, fibrotic disorder) by inhibiting cell proliferation and cytosolic protein synthesis.

### Methods of Treatment

In some aspects, methods provided in the present disclosure are drawn to treating a disease or disorder by administering to a subject in need thereof an antagonist of nicotinamide adenine dinucleotide kinase 2.

### Nicotinamide Adenine Dinucleotide Kinase 2 (NADK2)

In some aspects, methods and compositions provided in the present disclosure comprise an antagonist of nicotinamide adenine dinucleotide kinase 2 (NADK2). NADK2 is a mitochondrial enzyme that phosphorylates nicotinamide adenine dinucleotide (NAD+) to produce NADP⁺. NAD⁺ and NADH and NADP⁺ and NADPH may be used interchangeably herein. Because NADP⁺ is membrane impermeable, mitochondrial NADP⁺ is separate from cytosolic NADP₊ produced by nicotinamide adenine dinucleotide kinase 1 (NADK1). As demonstrated herein, NADP⁺ produced from NADK2 is required for cell proliferation, proline biosynthesis, and cytosolic protein synthesis. Thus, antagonizing the activity of NADK2 (e.g., with an NADK2 antagonist) is an effective strategy for inhibiting cell proliferation, proline biosynthesis, and cytosolic protein synthesis.

NADK2 herein may be NADK2 expressed in any organism known in the art. NADK2 is conserved in human (Gene ID: 133686), mouse (Gene ID: 68646), rat (Gene ID: 365699), frog (Gene ID: 780144), non-human primates (Gene IDs: 704285, 461919), cow (Gene ID: 506968), zebrafish (Gene ID: 445071), chicken (Gene ID: 417438), dog (Gene ID: 612569), hamster (Gene ID: 101837077), horse (Gene ID: 100067696) and fish (Gene IDs: 108279376, 108900730, 109868343). In some embodiments, NADK2 is human NADK2.

Human NADK2 may be any human NADK2 sequence known in the art. Human NADK2 is alternatively spliced to produce 3 different isoforms. Human NADK2 isoform 1 (Q4G0N4-1) is 442 amino acids in length and is considered full-length. Human NADK2 isoform 2 (Q4G0N4-2) is 410 amino acids in length and is missing amino acids 288-319 from the NADK2 isoform 1 sequence. Human NADK2 isoform 3 (Q4G0N4-3) is 279 amino acids in length and is missing amino acids 1-163 from the NADK2 isoform 1 sequence.

In some embodiments, an antagonist of NADK2 is administered to a subject in need thereof. An antagonist is a compound or molecule that inhibits the activity of a protein. An antagonist of NADK2 may decrease NADK2 activity by 10%-100%, 20%-90%, 30%-80%, 40%-70%, or 50%-60%. In some embodiments, an antagonist of NADK2 may decrease NADK2 activity by 10%, 15%, 20%, 25%, 30%, 35%, 40%, 45%, 50%, 55%, 60%, 65%, 70%, 75%, 80%, 85%, 90%, 95%, or 100%.

An antagonist of NADK2 inhibits the activity of NADK2 directly or indirectly. A direct antagonist of NADK2 binds to NADK2 protein and inhibits its catalytic activity (e.g., by blocking the enzyme active site). An indirect antagonist of NADK2 inhibits the production of NADK2 protein (e.g., NADK2 transcription, NADK2 translation).

An antagonist of NADK2 may be any NADK2 antagonist known in the art (see, e.g., WO 2016/170348). Non-limiting examples of potential NADK2 antagonists include small organic compounds having a molecular weight of less than about 1,000 g/mol; nucleotide compounds including a guide RNA used in a clustered regularly interspaced short palindromic repeats (CRISPR/Cas) genome editing system, an antisense oligonucleotide, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), a short (or small) activating RNA (saRNA) or a combination thereof; an anti-NADK2 antibody; and an anti-NADK2 nucleic acid aptamer.

In some embodiments, an antagonist of NADK2 is a guide RNA (gRNA) used in a CRISPR/Cas genome editing system. CRISPR/Cas genome editing is well-known in the art (see, e.g., Wang et al., Ann. Rev. Biochem., 2016, 85: 227-264; Pickar-Oliver and Gersbach, Nature Reviews Molecular Cellular Biology, 2019, 20: 490-507; Aldi, Nature Communications, 2018, 9: 1911). In some embodiments, a gRNA antagonist of NADK2 knocks out (removes) NADK2 from the genome, decreases expression of NADK2 from the gnome, decreases NADK2 enzyme activity, or a combination thereof. A gRNA antagonist of NADK2 may be 1-10, 2-9, 3-8, 4-7, or 5-6 gRNAs. In some embodiments, a gRNA antagonist of NADK2 may be 1, 2, 3, 4, 5, 6, 7, 8, 9, or 10 or more gRNAs.

A subject in need thereof may be administered one antagonist of NADK2 or multiple antagonists of NADK2. When multiple antagonists of NADK2 are administered, the multiple antagonists may have the same mechanism of action (e.g., inhibiting NADK2 expression, inhibiting NADK2 enzymatic activity), different mechanisms of action, or a combination thereof. In some embodiments, 1-10, 2-9, 3-8, 4-7, or 5-6 antagonists of NADK2 are administered to a subject in need thereof. In some embodiments, 1, 2, 3, 4, 5, 6, 7, 8, 9, or 10 or more antagonists of NADK2 are administered to a subject in need thereof. When multiple antagonists of NADK2 are administered to a subject, they may be administered in the same administration or in multiple administrations.

### Cancer

In some aspects, the present disclosure provides a method of treating a cancer. Treating a cancer may be killing cancer cells, inhibiting the proliferation of cancer cells, inhibiting the growth of cancer cells, inhibiting the metastasis of cancer cells, or any other measure of treating cancer known in the art. A cancer treated with a method provided herein may be a primary cancer or a secondary cancer. A primary cancer is a cancer that is confined to the original location where the cancer began (e.g., breast, colon, etc.), and a secondary cancer is a cancer that originated in a different location and metastasized. A cancer treated with a method provided herein may be a first occurrence of the cancer or may be a subsequent occurrence of the cancer (relapsed or recurrent cancer).

In some embodiments, a method provided herein includes treating a cancer characterized as having an isocitrate dehydrogenase 2 (IDH2) mutation. Characterized as having means that a mutation (e.g., IDH2 mutation) has been detected in the cancer. IDH2 is a mitochondrial enzyme produced by expression of the IDH2 gene. IDH2 catalyzes the oxidative decarboxylation of isocitrate to alpha-ketoglutarate (αKG, also known as 2-oxoglutarate) as part of the tricarboxylic acid (TCA) cycle that produces energy in the form of adenine trinucleotide phosphate (ATP). Because αKG is membrane impermeable, mitochondrial αKG is separate from cytosolic αKG produced by isocitrate dehydrogenase 1 (IDH1).

IDH2 herein may be IDH2 from any organism known in the art. IDH2 is expressed in human (Gene ID: 3418), mouse (Gene ID: 269951), rat (Gene ID: 361596), pig (Gene ID: 397603), frog (Gene ID: 448026), non-human primates (Gene IDs: 701480, 453645), cow (Gene ID: 327669), zebrafish (Gene ID: 386951), chicken (Gene ID: 431056), dog (Gene ID: 479043), and fish (Gene IDs: 100194639, 100304677, 105025672). In some embodiments, IDH2 is human IDH2.

Human IDH2 may be any human IDH2 sequence known in the art. Human IDH2 is alternatively spliced to produce 2 different isoforms. Human IDH2 isoform 1 (P48735-1) is 452 amino acids in length and is considered full-length. Human IDH2 isoform 2 (P48735-2) is 400 amino acids in length and is missing amino acids 1-52 from the IDH2 isoform 1 sequence.

An IDH2 mutation may be any mutation known in the art that is associated with cancer. Associated with cancer means that an IDH2 mutation has been detected in a cancer cell. IDH2 is mutated in 1.39% of all cancers, with acute myeloid leukemia, breast invasive ductal carcinoma, colon adenocarcinoma, lung adenocarcinoma, and oligodendroglioma having the greatest prevalence of IDH2 mutations (31).

An IDH2 mutation may be a gain-of-function mutation or a loss-of-function mutation. A gain-of-function IDH2 mutation is a mutation that confers a stronger (e.g., higher activity, more constitutive activity, etc.) enzymatic function or an additional enzymatic function to an IDH2 protein compared to wild-type IDH2. A loss-of-function IDH2 mutation is a mutation that confers a weaker (e.g., lower activity, less constitutive activity, etc.) enzymatic activity or losing an enzymatic function that is expressed compared to wild-type IDH2.

An IDH2 mutation may be any mutation known in the art. Non-limiting examples of IDH2 mutations include R172S, exon 4 mutation, a codon 140 missense mutation, R140Q, a codon 172 missense mutation, R172K, an amplification of IDH2, a loss of IDH2, R172W, R172M, R140W. R172G, V305M, H384Q, T350P, R172T, V355I, K155N, A416V, W21S, X39 splice, R159H, A347T, D390Y, D259N, A370T, and A174T.

In some embodiments, a cancer characterized as having an IDH2 mutation has a combination of IDH2 mutations known in the art. In some embodiments, a cancer characterized as having an IDH2 mutation has 1-10, 2-9, 3-8, 4-7, or 5-6 mutations. In some embodiments, a cancer characterized as having an IDH2 mutation has 1, 2, 3, 4, 5, 6, 7, 8, 9, or 10 or more mutations.

In some embodiments, an IDH2 mutation produces a mutant IDH2 protein having a neomorphic activity. A neomorphic activity is an enzymatic function that the mutant IDH2 protein possesses and does not normally have or has at a higher level than a wild-type protein. Mutations in IDH2 may contribute to cancer through production of 2-hydroxyglutarate (2HG) from αKG. Thus, in some embodiments, mutations in IDH2 that confer a neomorphic (e.g., gain-of-function) activity to the IDH2 enzyme produce increased levels of 2HG compared to wild-type IDH2 enzyme (32). Therefore, in some embodiments, a cancer that has an IDH2 mutation has increased levels of 2HG relative to a reference value. In some embodiments, a cancer that has an IDH2 mutation has decreased levels of αKG relative to a reference value. Levels of 2HG and αKG may be measured by any method known in the art. Non-limiting examples of methods for measuring levels of 2HG and αKG include: gas chromatography-mass spectrometry (GC-MS), liquid chromatography-mass spectrometry (LC-MS), colorimetric assay, and fluorometric assays.

A reference value may be from a cell characterized as not having an IDH2 mutation, a non-cancerous cell, or a cell that is not contacted with an antagonist of NADK2. A non-cancerous cell is a cell that does not possess a mutation associated with cancer. A mutation associated with cancer may be any mutation known in the art to occur in cancer cells.

In some embodiments, a cancer provided herein is characterized as not having an isocitrate dehydrogenase (IDH1) mutation. IDH1 catalyzes the oxidative decarboxylation of isocitrate to αKG in the cytosol of a cell as part of the TCA cycle that produces energy in the form of ATP.

In some embodiments, a cancer treated with a method provided herein is an adenocarcinoma. An adenocarcinoma is a cancer that forms in epithelial cells that produce fluids or mucus. An adenocarcinoma may be any adenocarcinoma known in the art. Non-limiting examples of adenocarcinomas include colon adenocarcinoma, lung adenocarcinoma, high grade ovarian serous adenocarcinoma, colorectal adenocarcinoma, rectal adenocarcinoma, prostate adenocarcinoma, breast adenocarcinoma, or a combination thereof.

In some embodiments, a cancer treated with a method provided herein is a carcinoma. Carcinoma is the most common type of cancer and is formed by epithelial cells. A carcinoma may be any carcinoma known in the art. Non-limiting examples of carcinoma include: breast invasive ductal carcinoma, intrahepatic cholangiocarcinoma, endometrial endometrioid carcinoma, bladder urothelial carcinoma, endometrial carcinoma, squamous cell lung carcinoma, or a combination thereof.

In some embodiments, a cancer is selected from acute myeloid leukemia, oligodendroglioma, myelodysplastic syndrome, cutaneous melanoma, glioblastoma multiforme, angioimmunoblastic T-cell lymphoma, acute monoblastic and monocytic leukemia, or a combination thereof.

### Fibrotic Disorder

In some aspects, the present disclosure provides a method of treating a fibrotic disorder by administering to a subject in need thereof an antagonist of NADK2 in an amount effective to treat the fibrotic disorder. A fibrotic disorder is a disorder in which extracellular matrix molecules uncontrollably and progressively accumulate in affected tissues and organs, causing their ultimate failure. Fibrosis is a predominant feature of the pathology of a wide range of diseases across numerous organ systems, and fibrotic disorders are estimated to contribute to up to 45% of all-cause mortality in the United States. Despite this prevalence of fibrotic disorders, effective therapies are limited.

In some embodiments, a fibrotic disorder that is treated with a method provided herein is characterized by increased levels of an extracellular matrix (ECM) protein. An ECM protein is a protein in a three-dimensional network of extracellular macromolecules and minerals that exists between cells. An ECM protein herein may be any ECM protein known the in art. Non-limiting examples of ECM proteins include: collagen, elastin, fibronectin, and laminin. More than one ECM protein may also have increased levels in a fibrotic disorder treated herein. In some embodiments, a fibrotic disorder is characterized by increased levels of 1-10, 2-9, 3-8, 4-7, or 5-6 ECM proteins. In some embodiments, a fibrotic disorder is characterized by increased levels of 1, 2, 3, 4, 5, 6, 7, 8, 9, or 10 or more ECM proteins.

In some embodiments, a fibrotic disorder that is treated with a method provided herein is characterized by increased levels of a collagen protein. Collagens are the most abundant protein in the ECM and the human body. Collagen is produced in cells and exocytosed in precursor form (procollagen) which is then cleaved and assembled into mature collagen extracellular. Collagen proteins may be divided into several families based on the types of structures that they form, including, but not limited to: fibrillar (Types I, II, III, V, and XI collagens), facit (Types IX, XII, and XIV collagens), short chain (Types VIII and X collagens), basement membrane (Type IV), and other structures (Types VI, VII, and XIII).

Extracellular matrix proteins require amino acids, such as proline, that confer structural rigidity to fold into and maintain the proper architecture. In addition to its role in promoting cell proliferation discussed above, NADP⁺ produced by NADK2 is also required for proline biosynthesis in a nutrient-deficient environment. A nutrient-deficient environment lacks sufficient levels of one or more nutrients to allow cellular processes (e.g., cell proliferation, protein synthesis, proline biosynthesis). Proline is produced by the conversion of glutamate to pyrroline-5-carboxylate (P5C) by pyrroline-5-carboxylate synthase (P5CS), which requires NADPH produced by NADK2. P5C is further reduced to proline by mitochondrial pyrroline-5-carobxylate reductases (PYCR1 and PYCR2). Thus, contacting NADK2 with an antagonist reduces proline biosynthesis in a nutrient-deficient environment by inhibiting the conversion of glutamate to P5C.

As described above, NADK2 and P5CS are required for proline biosynthesis and fibrosis in a nutrient-deficient environment. Thus, in some embodiments, a fibrotic disorder treated with a method provided herein is characterized by increased levels of NADK2, increased levels of P5CS, or increased levels of NADK2 and increased levels of P5CS relative to a known reference value.

A reference value may be a normal cell, a cell that is not contacted with an antagonist of NADK2, or a cell in a nutrient-replete environment. A normal cell is a cell that is not associated with fibrosis and does not have an increased level of NADK2, P5CS, or NADK2 and P5CS. A nutrient-replete environment has sufficient levels of one or more nutrients to allow cellular processes (e.g., cell proliferation, protein synthesis, proline biosynthesis).

A fibrotic disorder may be any fibrotic disorder known in the art. Non-limiting examples of fibrotic disorders include: idiopathic pulmonary fibrosis (IPF), hepatic fibrosis, systemic sclerosis, sclerodermatous graft vs. host disease, nephrogenic systemic fibrosis, radiation-induced fibrosis, cardiac fibrosis, kidney fibrosis, or a combination thereof. Treating a fibrotic disorder may mean decreased proline synthesis, decreased synthesis of ECM proteins, decreased deposition of ECM proteins, reduction of existing depositions of ECM proteins, or a combination thereof.

Proline synthesis may be measured by any method known in the art including, but not limited to: isotopologue labeling followed by GC-MS quantification, isotopologue labeling following by LC-MS quantification, ninhydrin staining, and colorimetric assays. Any isotopologue known in the art may be used in methods of quantifying proline, including but not limited to: [¹³C], [¹⁶O], [¹⁷O], [¹⁸O], [²H], [¹⁵N], [2,3,3-²H₃]serine, [U-¹³C], [U-¹⁶O], [U-¹⁷O], [U-¹⁸O], [U-²H], and [U-¹⁵N].

Extracellular matrix protein may be measured by any method known in the art including, but not limited to: protein staining, isobaric demethylated leucine (DiLeu) labeling and quantification, mass spectrometry, reversed phase liquid chromatography, second harmonic generation (SHG) microscopy, and strong cation exchange chromatography. In some embodiments, ECM proteins are measured by protein staining. Non-limiting examples of protein staining of ECM proteins include: Picrosirius Red staining. Masson's Trichrome staining, and hematoxylin and eosin staining.

### Subjects

Methods provided herein may be used to treat a subject in need thereof. A subject in need thereof may have any disease or disorder provided herein including, but not limited to, a cancer (e.g., adenocarcinoma, carcinoma, leukemia, glioma) and a fibrotic disease (e.g., pulmonary fibrosis, liver fibrosis, kidney fibrosis). A subject may have one or more diseases or disorders provided herein. In some embodiments, a subject has 1-10 diseases or disorders, 2-9 diseases or disorders, 3-8 diseases or disorders, 4-7 diseases or disorders, or 5-6 diseases or disorders. In some embodiments, a subject has 1, 2, 3, 4, 5, 6, 7, 8, 9, or 10 or more diseases or disorders provided herein.

In some embodiments, a subject is administered an effective amount of an antagonist of NADK2 to treat a disease or disorder. An effective amount of an antagonist of NADK2 is any amount that decreases cell proliferation, decreases cell survival, decreases protein synthesis, decreases proline biosynthesis, decreases ECM protein deposition, decreases fibrosis, or a combination thereof.

An effective amount of an antagonist of NADK2 will vary based on factors that are known to a person skilled in the art, including, but not limited to: age of a subject, height of a subject, weight of a subject, pre-existing conditions, stage of a disease or disorder, other treatments or medications that a subject is being administered, or a combination thereof. In some embodiments, an effective amount of an antagonist of NADK2 is 1 μg/kg-1,000 mg/kg, 10 μg/kg-100 mg/kg, 100 μg/kg-10 mg/kg, or 500 μg/kg-1 mg/kg. In some embodiments, an effective amount of an antagonist of NADK2 is 1 μg/kg, 10 μg/kg, 25 μg/kg, 50 μg/kg, 75 μg/kg, 100 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1 mg/kg, 10 mg/kg, 25 mg/kg, 50 mg/kg, 75 mg/kg, 100 mg/kg, 200 mg/kg, 250 mg/kg, 300 mg/kg, 350 μg mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1,000 mg/kg.

In some embodiments, a subject is a vertebrate. A vertebrate may be any vertebrate known in the art including, but not limited to: a human, a rodent (e.g., mouse, rat, hamster), a non-human primate (e.g., Rhesus monkey, chimpanzee, orangutan), a pet (e.g., dog, cat, ferret), a livestock animal (e.g., pig, cow, sheep, chicken), or a fish (zebrafish, catfish, perch).

An antagonist of NADK2 may be administered to a subject by any method known in the art. Non-limiting examples of methods for administering an antagonist of NADK2 include: injection (e.g., intravenous, intramuscular, intraarterial), inhalation (e.g., by nebulizer, by inhaler), ingestion (e.g., oral, rectal, vaginal), sublingual or buccal dissolution, ocular placement, otic placement, and absorbed through skin (e.g., cutaneously, transdermally).

### Methods for Use

Methods provided herein may be used in vitro (e.g., in a cultured cell) or in vivo (e.g., in a subject) to antagonize NADK2. Because NADK2 is required for proline biosynthesis, cytosolic protein synthesis, and cell proliferation in a nutrient-deficient environment, methods provided herein may be used to inhibit protein synthesis and cell proliferation in vitro or in vivo.

### Inhibiting Protein Synthesis

As described above, NADK2 is required for proline biosynthesis in nutrient-deficient environments. Proline that is produced in mitochondria is utilized in protein synthesis, particularly for proteins that require structural rigidity and specific conformations (e.g., ECM proteins). Thus, in some aspects, methods provided herein may be used to inhibit protein synthesis. These methods may be used to inhibit protein synthesis in vitro (e.g., in cell culture) or in vivo (e.g., in a subject).

When methods provided herein for inhibiting protein synthesis are in vivo in a subject in need thereof, they may be used to treat a disease or disorder associated with increased or aberrant protein synthesis. Aberrant protein synthesis may be synthesis of mutant protein, synthesis of a pathologic protein, or a combination thereof. A pathologic protein may be a protein that malfunctioned protein folding (compared to its wild-type counterpart).

In some embodiments, when methods provided herein for inhibiting protein synthesis are in vivo in a subject in need thereof, the subject is on a restrictive diet. A restrictive diet decreases and/or increases the consumption of specific foods or limits nutrient intake to a certain number of calories (also known as kilocalories). Non-limiting examples of foods that may be decreased on a restrictive diet include refined grains (e.g., fried rice, granola, biscuits, sweet rolls, muffins, scones, coffee bread, doughnuts, cheese bread), sweets (e.g., cookies, cakes, candy, ice cream), snacks (e.g., chips, pretzels, crackers), certain proteins (e.g., duck, goose, bacon, sausage, hot dogs, cold cuts, nuts, nut butters), dairy (e.g., whole milk, cream, whole milk yogurt, whole milk cheese), beverages (e.g., alcohol, carbonated beverages with sugar, juices with added sugar), or any combination thereof. Non-limiting examples of foods that may be increased on a restrictive diet include fruits (e.g., berries, apples, citrus), vegetables (e.g., green beans, peas, carrots, lettuce, cabbage), whole grains (e.g., rice, popcorn, bread, pasta, cereal), natural sweeteners (e.g., honey, agave syrup, maple syrup), lean proteins (e.g., chicken, turkey, fish, beans, beans, legumes, eggs), dairy (e.g., reduced fat or non-fat milk, reduced fat or non-fat cheese, reduced fat or non-fat yogurt), beverages (e.g., coffee, tea, water), or some combination thereof. Non-limiting examples of certain numbers of calories that may be consumed daily on a restrictive diet include: 800 calories-1900 calories, 900 calories-1800 calories, 1000 calories-1700 calories, 1100 calories-1600 calories, 1200 calories-1500 calories, 1300 calories-1400 calories. A restrictive diet may be any restrictive diet known in the art including, but not limited to: 5:2 diet, Body for Life, cookie diet, The Hacker's Diet, Nurtisystem® diet, Weight Watchers® diet, inedia, KE diet, Atkins® diet, Dukan diet, South Beach Diet®, Stillman diet, Beverly Hills® diet, cabbage soup diet, grapefruit diet, monotrophic diet, Subway® diet, juice fasting, Master Cleanse®, DASH diet, diabetic diet, elemental diet, ketogenic diet, liquid diet, low-FODMAP diet, vegetarian diet, pescatarian diet, vegan diet, and soft diet.

Any disease or disorder associated with increased or aberrant protein synthesis known in the art may be treated with methods provided herein. Non-limited examples of diseases or disorders associated with increased or aberrant protein synthesis include: fibrosis, Alzheimer's disease, Parkinson's disease, Huntington's disease, cystic fibrosis, Gaucher's disease, amyloidosis, multiple system atrophy, and prion diseases (e.g., kuru, fatal familial insomnia, Creutzfeldt-Jakob Disease (CJD), variant Creutzfeldt-Jakob Disease (vCJD)).

Cellular protein synthesis may be measured by any method known in the art. Non-limiting examples of measuring protein synthesis include: radioactive isotope labeling (e.g., ³H-phenylalanine, ³⁵S-methionine), stable isotope labeling (e.g., ¹⁵N-lysine, ¹³C-leucine, ring-¹³C₆-phenylalanine), puromycin Surface Sensing of Translation (SUnSET) labeling, Western blot, GC-MS, LC-MS, and protein staining.

### Inhibiting Cell Proliferation

As described above, NADK2 is required for cell proliferation in a nutrient-deficient environment (e.g., nutrient-deficient cell culture media). Thus, in some aspects, methods provided herein may be used to inhibit cell proliferation. These methods may be used to inhibit cell proliferation in vitro (e.g., in cell culture) or in vivo (e.g., in a subject).

When methods provided herein for inhibiting cell proliferation are in vivo in a subject in need thereof, they may be used to treat a disease or disorder associated with increased cell proliferation. Any disease or disorder associated with increased cell proliferation known in the art may be treated with methods provided herein. Non-limiting examples of diseases or disorders associated with increased cell proliferation include: cancer, ataxia telangiectasia, xeroderma pigmentosum, autoimmune lymphoproliferative syndrome (types I and II), systemic lupus erythematosus, polycythemia vera, familial hemophagocytic lymphohistiocytosis, Niemann-Pick disease, osteoporosis, adenovirus infection, baculovirus infection, Epstein-Barr virus infection, Herpes virus infection, poxvirus infection, Down's syndrome, progeria, and atherosclerosis.

Cell proliferation may be an increase in cell metabolites or an increase in cell numbers. Cell proliferation may be measured or monitored by any method known in the art. Non-limiting methods of cell proliferation include: bromodeoxyuridine (BrdU) incorporation, 5-Ethynyl-2′-deoxyuridine (EdU) incorporation, 3-(4,5-dimethylthiazol-2-yl)-2,5-diphenyltetrazolim bromide (MTT) salt cleavage, (2,3-bis-(2-methoxy-4-nitro-5-sulfophenyl)-2H-tetrazolium-5-carboxanilide) (XTT) salt cleavage, (3-(4,5-dimethylthiazol-2-yl)-5-(3-carboxymethoxyphenyl)-2-(4-sulfophenyl)-2H-tetrazolium) (MTS) salt cleavage, (2-(2-methoxy-4-nitrophenyl)-3-(4-nitrophenyl)-5-(2,4-disulfophenyl)-2H-tetrazolium) (WST-8) salt cleavage, and Ki67 nuclear protein antibody labeling.

### Compositions

The present disclosure demonstrates that NADK2 is required for proline biosynthesis and cell proliferation in a nutrient-deficient environment, including a nutrient-deficient cell culture medium. Cells contacted with an antagonist of NADK2 in nutrient-deficient cell culture medium will have reduced proliferation due to decreased proline biosynthesis. Thus, in some aspects, the present disclosure provides a composition comprising (i) nutrient-deficient cell culture medium; and (ii) an antagonist of NADK2. This composition may be used in methods of treating a subject having a disease or disorder (e.g., cancer, fibrotic disorder).

Nutrient-deficient cell culture medium is cell culture medium deficient in one or more nutrients required for cellular processes, including but not limited to: amino acids, vitamins, and ions. Deficient in one or more amino acids means that the cell culture medium does not contain sufficient levels of one or more amino acids to support cellular processes. The cellular processes that are not supported in nutrient-deficient cell culture medium may be cell proliferation, survival, proline biosynthesis, ECM protein, ECM deposition, or a combination thereof.

Nutrient-deficient cell culture medium may be deficient in any amino acid including, but not limited to, arginine, alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or any combination thereof. In some embodiments, nutrient-deficient cell culture medium is deficient in 1-20, 2-19, 3-18, 4-17, 5-16, 6-15, 7-14, 8-13, 9-12, or 10-11 amino acids. In some embodiments, nutrient-deficient cell culture medium is deficient in 1, 2, 3, 4, 5, 6, 7, 8, 9, 10, 11, 12, 13, 14, 15, 16, 17, 18, 19, or 20 amino acids. In some embodiments, nutrient-deficient cell culture medium is deficient in proline.

In some embodiments, a composition provided herein further comprises a population of cells. A population of cells may be a homogeneous population composed of the same cell type or a heterogenous population composed of a mixture of cell types. A population of cells may be in vitro (e.g., in cell culture medium) or in vivo (e.g., in a subject). In some embodiments, a population of cells is obtained from a subject and maintained in vitro (e.g., in cell culture medium).

A population of cells may contain any number of cells including, but not limited to: 5 cells-100 cells, 50 cells-500 cells, 250 cells-1,000 cells, 500 cells-10,000 cells, 5,000 cells-100,000 cells, 50,000 cells-1,000,000 cells, 500,000 cells-10,000,000 cells, 1,000,000-1,000,000,000 cells, 5,000,000 cells-10,000,000,000 cells or more.

In some embodiments, the population of cells comprises cancer cells. The cancer cells may be derived from any cancer provided herein or a combination of cancers provided herein. In some embodiments, a population of cancer cells express a mutant IDH2 protein. A mutant IDH2 protein may be any mutant IDH2 protein provided herein.

In some embodiments, a mutant IDH2 protein in a cancer cell population provided herein has a neomorphic enzymatic activity. In some embodiments, the neomorphic enzymatic activity is a reduction of αKG to 2HG. Thus, in some embodiments, a cancer cell population expressing a mutant IDH2 protein having a neomorphic activity contains increased levels of 2HG relative to a known reference value. In some embodiments, a cancer cell population expressing a mutant IDH2 protein having a neomorphic activity contains reduced levels of 2HG relative to a known reference value.

A nutrient-deficient cell culture medium provided herein may contain one or more additives. Additives are exogenous compounds that are added to a nutrient-deficient medium. An additive may be any compound known in the art to be added to cell medium. Non-limiting examples of classes of compounds that are added to cell medium include: antibiotics (e.g., streptomycin, penicillin, ampicillin, kanamycin), serum (e.g., bovine serum albumin, human serum albumin, fetal bovine serum), amino acids (e.g., arginine, alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine), inorganic salt (e.g., ammonium molybdate, ammonium metavandate, calcium chloride, cupric sulfate, ferric nitrate, ferrous sulfate, manganese sulfate, magnesium chloride, magnesium sulfate, nickel chloride, potassium chloride, sodium metasilicate, sodium selenite, sodium phosphate dibasic, sodium phosphate monobasic, stannous chloride, zinc sulfate), vitamins (e.g., biotin, choline chloride, folic acid, myo-inositol, niacinamide, pantothenic acid, pyridoxal, pyridoxine, riboflavin, thiamine, vitamin B12), buffers (e.g., glucose, HEPES, hypoxanthine, linoleic acid, Phenol Red, putrescine, pyruvic acid, thioctic acid, thymidine, sodium bicarbonate).

In some embodiments, nutrient-deficient cell culture medium contains serum, penicillin, and streptomycin. The concentration of serum, penicillin, and streptomycin may be any concentration in cell culture medium known in the art. In some embodiments, nutrient-deficient cell culture medium contains 1%-30%, 2%-29%, 3%-28%, 4%-27%, 5%-26%, 6%-25%, 7%-24%, 8%-23%, 9%-22%, 10%-21%, 11%-20%, 12%-19%, 13%-18%, 14%-17%, or 15%-16% serum. In some embodiments, nutrient-deficient cell culture medium contains 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% serum. In some embodiments, nutrient-deficient cell culture medium contains 10 units/mL-150 units/mL, 20 units/mL-140 units/mL, 30 units/mL-130 units/mL, 40 units/mL-120 units/mL, 50 units/mL-110 units/mL, 60 units/mL-100 units/mL, or 70 units/mL-90 units/mL penicillin. In some embodiments, nutrient-deficient cell culture medium contains 10 units/mL, 20 units/mL, 30 units/mL, 40 units/mL, 50 units/mL, 60 units/mL, 70 units/mL, 80 units/mL, 90 units/mL, 100 units/mL, 110 units/mL, 120 units/mL, 130 units/mL, 140 units/mL, or 150 units/mL penicillin. In some embodiments, nutrient-deficient cell culture medium contains 10 μg/mL-150 μg/mL, 20 μg/mL 140 μg/mL, 30 μg/mL-130 μg/mL, 40 μg/mL-120 μg/mL, 50 μg/mL-110 μg/mL, 60 μg/mL-100 μg/mL, or 70 μg/mL-90 μg/mL streptomycin. In some embodiments, nutrient-deficient cell culture medium contains 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, or 150 μg/mL streptomycin.

### EXAMPLES

### Example 1: NADK2 is Required to Maintain Mitochondrial 2-Hydroxyglutrate Levels

The data in this Example demonstrates that NADK2 is required to maintain mitochondrial NADPH and mitochondrial 2-hydroxyglutrate (2-HG) in cells expressing mutant IDH2.

Mammalian cells depend on the inter-conversion of nicotinamide adenine dinucleotide phosphate (NADP) molecules between the oxidized (NADP⁺) and reduced (NADPH) forms to support reductive biosynthesis and to maintain cellular antioxidant defense. NADP⁺ and NADPH molecules (also referred to as “NADP(H)”) are unable to cross subcellular membranes (1, 2). As a result, cellular pools of NADP(H) are compartmentalized. In the cytosol, NADP(H) is derived from nicotinamide adenine dinucleotide [(NAD)H] by NAD kinase 1 (NADK1). Cytosolic NADPH acts as a substrate in fatty acid biosynthesis, and as the reducing equivalent required to regenerate reduced glutathione (GSH) and thioredoxin for antioxidant defense. Mitochondria host a number of biosynthetic activities critical for cellular metabolism but are also major sites for reactive oxygen species (ROS) generation. Mammalian mitochondrial NAD kinase 2 (NADK2) converts NAD(H) to NADP(H) through phosphorylation (3).

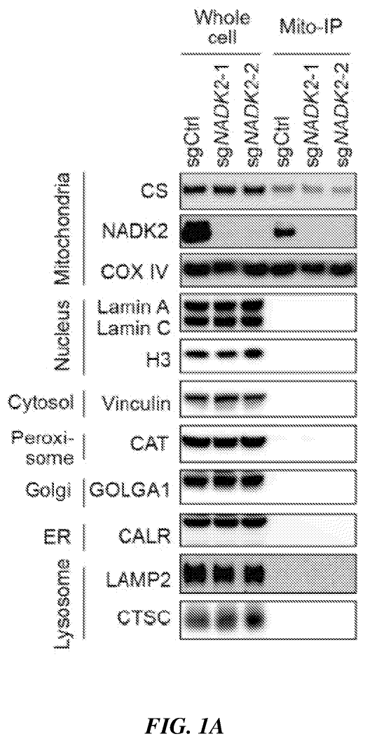

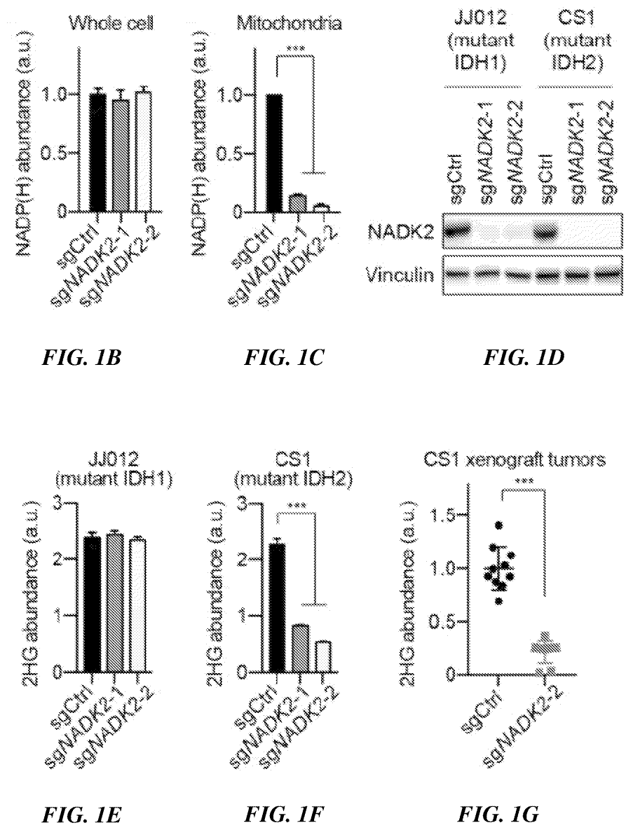

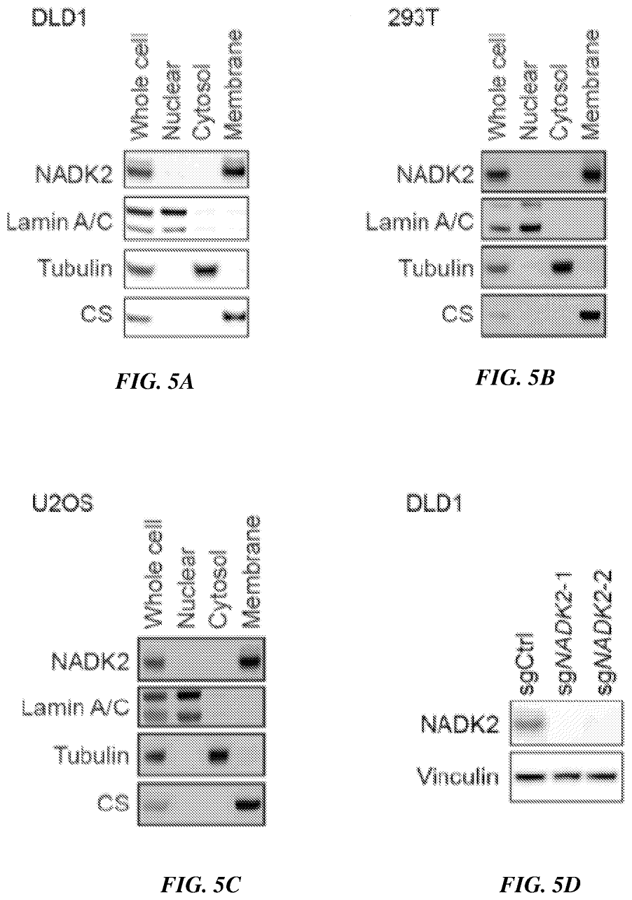

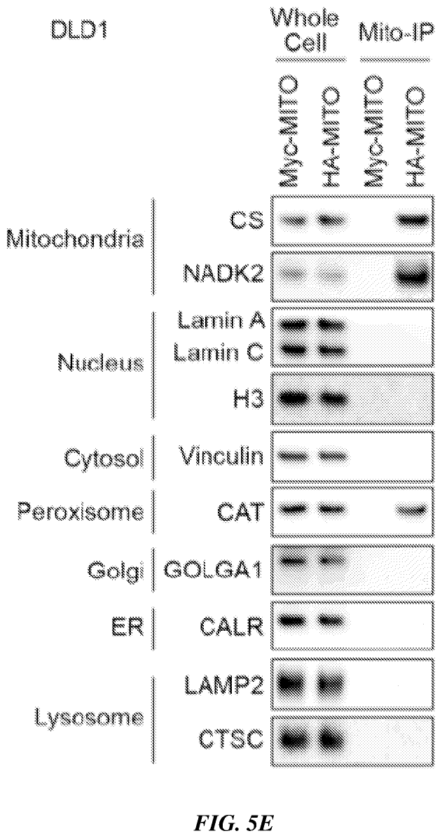

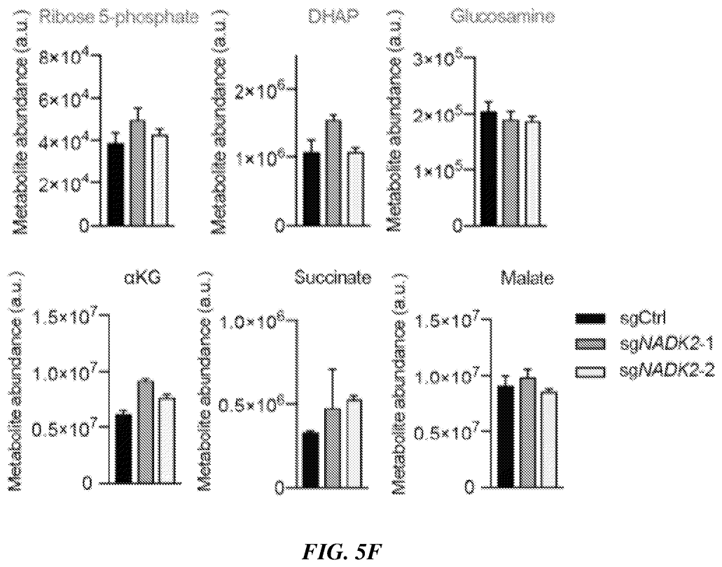

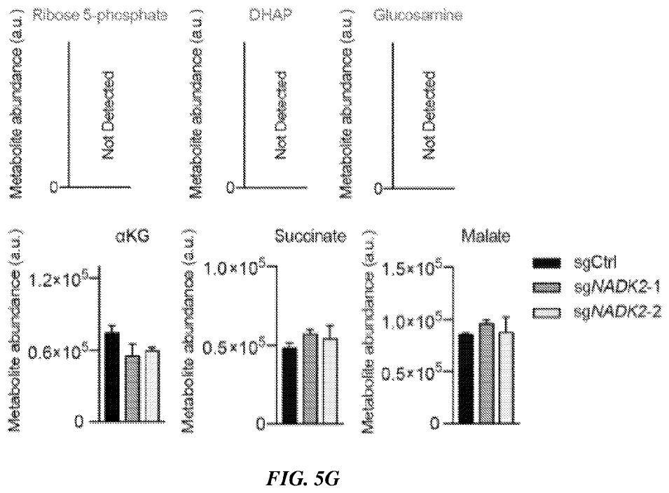

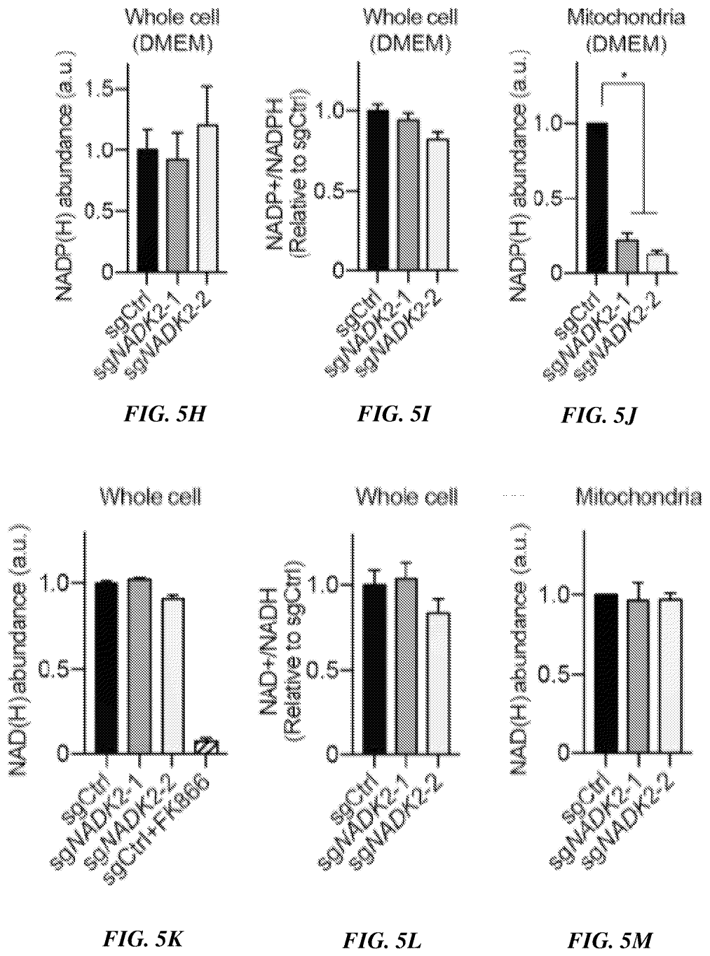

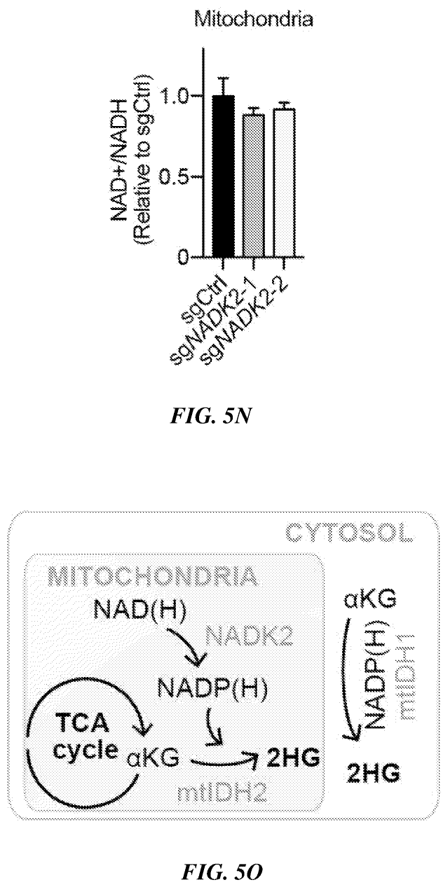

Using subcellular fractionation, it was confirmed that NADK2 purified in the membrane-associated fraction in cultured human cell lines (FIGS. 5A-5C). Mitochondria immunopurification (Mito-IP, 4, 5) from DLD1 cells following CRISPR-Cas9 deletion of NADK2 (FIG. 5D) resulted in a metabolomic profile consistent with mitochondrial metabolism, and metabolites known to be excluded from the mitochondrial compartment were minimally detected (FIGS. 1A; 5E-5G; Tables 1A-1G). A full list of all detected metabolites was annotated and included in Tables 1A-1G, including a picricidin treatment condition (sgCtrl DLD1-OMP25HA cells treated with 5 μM piericidin for 2 hours before performing Mito-IP) that validated the Mito-IP method. For example, piericidin treatment specifically increased glutamate and NADH levels in the mitochondria, but not in the whole cell samples. NADP(H) levels were examined in immunopurified mitochondria using an adapted enzyme cycling assay (6). Although total NADP(H) abundance or NADP+ to NADPH ratio were not changed at a whole cell level upon NADK2 loss as previously reported (6, 7), mitochondrial NADP(H) abundance was reduced by more than 80% (P<0.001) in NADK2 knockout cells (FIGS. 1B-1C; 5H-5J). NAD(H) abundance or NAD+ to NADH ratio were not altered by NADK2 knockout in whole cells or in mitochondria (FIGS. 5K-5N).

TABLE 1A
Metabolites results

| | Super | Sub | | |
|---|---|---|---|---|
| Metabolite Name | Pathway | Pathway | Formula | Identification |
| 4-hydroxyproline¹ | Amino Acid | Urea cycle; | C5H9NO3 | MS2; RT |
| | | Arginine and | | |
| | | Proline | | |
| | | Metabolism | | |
| 6-phosphogluconate¹ | Carbohydrate | Pentose Phosphate | C6H13O10P | RT |
| | | Pathway | | |
| aconitate¹ | Energy | TCA Cycle | C6H6O6 | RT |
| alanine¹ | Amino Acid | Alanine and | C3H7NO2 | RT |
| | | Aspartate | | |
| | | Metabolism | | |
| arginine¹ | Amino Acid | Urea cycle; | C6H14N4O2 | RT |
| | | Arginine and | | |
| | | Proline | | |
| | | Metabolism | | |
| betaine¹ | Amino Acid | Glycine, Serine | C5H11NO2 | RT |
| | | and Threonine | | |
| | | Metabolism | | |
| carnitine¹ | Lipid | Carnitine | C7H15NO3 | RT |
| | | Metabolism | | |
| creatine phosphate¹ | Amino Acid | Creatine | C4H10N3O5P | RT |
| | | Metabolism | | |
| creatinine¹ | Amino Acid | Creatine | C4H7N3O | RT |
| | | Metabolism | | |
| cytidine 5′- | Nucleotide | Pyrimidine | C9H16N3O14P3 | RT |
| triphosphate (CTP)¹ | | Metabolism, | | |
| | | Cytidine | | |
| | | containing | | |
| dihydroxyacetone | Carbohydrate | Glycolysis, | C3H7O6P | RT |
| phosphate (DHAP)¹ | | Gluconeogenesis, | | |
| | | and Pyruvate | | |
| | | Metabolism | | |
| formylpyruvate¹ | Other | Other | C4H4O4 | MS2 |
| fructose 6′-phosphate¹ | Carbohydrate | Glycolysis, | C6H13O9P | RT |
| | | Gluconeogenesis, | | |
| | | and Pyruvate | | |
| | | Metabolism | | |
| fumarate¹ | Energy | TCA Cycle | C4H4O4 | RT |
| glucosamine¹ | Carbohydrate | Aminosugar | C6H13NO5 | RT |
| | | Metabolism | | |
| glycerophosphorylcholine | Lipid | Phospholipid | C8H20NO6P | RT |
| (GPC)¹ | | Metabolism | | |
| glycine¹ | Amino Acid | Glycine, Serine | C2H5NO2 | MS2 |
| | | and Threonine | | |
| | | Metabolism | | |
| guanosine 5′- | Nucleotide | Purine | C10H15N5O11P2 | RT |
| diphosphate (GDP)¹ | | Metabolism, | | |
| | | Adenine | | |
| | | containing | | |
| guanosine 5′- | Carbohydrate | Nucleotide Sugar | C16H25N5O16P2 | RT |
| diphosphoglucose¹ | | | | |
| guanosine 5′- | Nucleotide | Purine | C10H14N5O8P | RT |
| monophosphate | | Metabolism, | | |
| (GMP)¹ | | Adenine | | |
| | | containing | | |
| guanosine 5′- | Nucleotide | Purine | C10H16N5O14P3 | RT |
| triphosphate (GTP)¹ | | Metabolism, | | |
| | | Guanine | | |
| | | containing | | |
| hydroxyphenyllactate¹ | Amino Acid | Tyrosine | C9H10O4 | MS2 |
| | | Metabolism | | |
| inosine 5′- | Nucleotide | Purine | C10H13N4O8P | RT |
| monophosphate | | Metabolism, | | |
| (IMP)¹ | | (Hypo)Xanthine/ | | |
| | | Inosine containing | | |
| isoleucine¹ | Amino Acid | Leucine, | C6H13NO2 | RT |
| | | Isoleucine and | | |
| | | Valine Metabolism | | |
| leucine¹ | Amino Acid | Leucine, | C6H13NO2 | RT |
| | | Isoleucine and | | |
| | | Valine Metabolism | | |
| lysine¹ | Amino Acid | Lysine Metabolism | C6H14N2O2 | RT |
| methionine¹ | Amino Acid | Methionine, | C5H11NO2S | RT |
| | | Cysteine, SAM | | |
| | | and Taurine | | |
| | | Metabolism | | |
| N6-(delta- | Other | Other | C10H13N5 | RT |
| isopentenyl)-adenine¹ | | | | |
| N-acetylalanine¹ | Amino Acid | Alanine and | C5H9NO3 | RT |
| | | Aspartate | | |
| | | Metabolism | | |
| N-acetylglycine¹ | Amino Acid | Glycine, Serine | C4H7NO3 | MS2; RT |
| | | and Threonine | | |
| | | Metabolism | | |
| N-acetylserine¹ | Amino Acid | Glycine, Serine | C5H9NO4 | MS2 |
| | | and Threonine | | |
| | | Metabolism | | |
| nicotinamide¹ | Cofactors and | Nicotinate and | C6H6N2O | RT |
| | Vitamins | Nicotinamide | | |
| | | Metabolism | | |
| nicotinamide adenine | Cofactors and | Nicotinate and | C21H30N7O17P3 | RT |
| ainucleotide | Vitamins | Nicotinamide | | |
| phosphate, reduced | | Metabolism | | |
| (NADPH)¹ | | | | |
| nicotinamide adenine | Cofactors and | Nicotinate and | C21H28N7O17P3 | RT |
| dinucleotide | Vitamins | Nicotinamide | | |
| phosphate (NADP+)¹ | | Metabolism | | |
| proline¹ | Amino Acid | Urea cycle; | C5H9NO2 | RT |
| | | Arginine and | | |
| | | Proline | | |
| | | Metabolism | | |
| ribose 5′-phosphate¹ | Carbohydrate | Pentose Phosphate | C5H11O8P | RT |
| | | Pathway | | |
| thymidine 5′- | Carbohydrate | Nucleotide Sugar | C16H26N2O16P2 | RT |
| diphospho-alpha-D- | | | | |
| glucose¹ | | | | |
| thymine¹ | Nucleotide | Pyrimidine | C5H6N2O2 | RT |
| | | Metabolism, | | |
| | | Thymine | | |
| | | containing | | |
| tryptophan¹ | Amino Acid | Tryptophan | C11H12N2O2 | RT |
| | | Metabolism | | |
| tyrosine¹ | Amino Acid | Tyrosine | C9H11NO3 | RT |
| | | Metabolism | | |
| uridine 5′-diphosphate | Nucleotide | Pyrimidine | C9H14N2O12P2 | RT |
| (UDP)¹ | | Metabolism, Uracil | | |
| | | containing | | |
| valine¹ | Amino Acid | Leucine, | C5H11NO2 | RT |
| | | Isoleucine and | | |
| | | Valine Metabolism | | |
| N-acetylglucosamine | Other | Other | C8H16NO9P | MS2 |
| 6-phosphate¹ | | | | |
| N-acetylthreonine¹ | Other | Other | C6H11NO4 | MS2 |
| galactitol¹ | Carbohydrate | Other | C6H14O6 | MS2 |
| alanylhistidine¹ | Amino Acid; | Histidine | C9H14N4O3 | MS2 |
| | Peptide | Metabolism | | |
| S- | Amino Acid | Methionine, | C14H20N6O5S | MS2; RT |
| adenosylhomocysteine | | Cysteine, SAM | | |
| (SAH)¹ | | and Taurine | | |
| | | Metabolism | | |
| serinylaspartate¹ | Amino Acid; | Other | C7H12N2O6 | MS2 |
| | Peptide | | | |
| adenine¹ | Nucleotide | Purine | C5H5N5 | MS2 |
| | | Metabolism, | | |
| | | Adenine | | |
| | | containing | | |
| folate¹ | Cofactors and | Folate Metabolism | C19H19N7O6 | MS2; RT |
| | Vitamins | | | |
| glycylaspartate¹ | Amino Acid; | Other | C6H10N2O5 | MS2 |
| | Peptide | | | |
| glyceraldehyde 3- | Other | Other | C3H7O6P | MS2 |
| phosphate¹ | | | | |
| 5- | Nucleotide | Pyrimidine | C11H15N5O3S | MS2; RT |
| methylthioadenosine | | Metabolism, Uracil | | |
| (MTA)¹ | | containing | | |
| N-acetyltaurine¹ | Other | Other | C4H9NO4S | MS2 |
| glucose 6′-phosphate¹ | Carbohydrate | Glycolysis, | C7H9N4O5P | MS2; RT |
| | | Gluconeogenesis, | | |
| | | and Pyruvate | | |
| | | Metabolism | | |
| nicotinamide adenine | Cofactors and | Nicotinate and | C21H29N7O14P2 | RT |
| dinucleotide, reduced | Vitamins | Nicotinamide | | |
| (NADH)² | | Metabolism | | |
| phenylalanine² | Amino Acid | Phenylalanine | C9H11NO2 | RT |
| | | Metabolism | | |
| pantothenate² | Cofactors and | Pantothenate and | C9H17NO5 | MS2; RT |
| | Vitamins | CoA Metabolism | | |
| phosphocholine² | Amino Acid | Glycine, Serine | C5H14NO4P | RT |
| | | and Threonine | | |
| | | Metabolism | | |
| 2-aminoadipate² | Amino Acid | Lysine Metabolism | C6H11NO4 | RT |
| creatine² | Amino Acid | Creatine | C4H9N3O2 | RT |
| | | Metabolism | | |
| flavin adenine | Cofactors and | Riboflavin | C27H33N9O15P2 | RT |
| dinucleotide (FAD)² | Vitamins | Metabolism | | |
| phosphoenolpyruvate | Carbohydrate | Glycolysis, | C3H5O6P | RT |
| (PEP)² | | Gluconeogenesis, | | |
| | | and Pyruvate | | |
| | | Metabolism | | |
| N-formylmethionine² | Amino Acid | Methionine, | C6H11NO3S | RT |
| | | Cysteine, SAM | | |
| | | and Taurine | | |
| | | Metabolism | | |
| N-acetylmethionine² | Amino Acid | Methionine, | C7H13NO3S | RT |
| | | Cysteine, SAM | | |
| | | and Taurine | | |
| | | Metabolism | | |
| nicotinamide adenine | Cofactors and | Nicotinate and | C21H27N7O14P2 | RT |
| dinucleotide (NAD+)² | Vitamins | Nicotinamide | | |
| | | Metabolism | | |
| N-acetylglutamate² | Amino Acid | Glutamate | C7H11NO5 | RT |
| | | Metabolism | | |
| allantoin² | Other | Other | C4H6N4O3 | MS2 |
| aspartate² | Amino Acid | Alanine and | C4H7NO4 | MS2; RT |
| | | Aspartate | | |
| | | Metabolism | | |
| taurine² | Amino Acid | Methionine, | C2H7NO3S | MS2 |
| | | Cysteine, SAM | | |
| | | and Taurine | | |
| | | Metabolism | | |
| adenosine 5′- | Nucleotide | Purine | C10H14N5O7P | MS2; RT |
| monophosphate | | Metabolism, | | |
| (AMP)² | | Adenine | | |
| | | containing | | |
| adenosine 5′- | Nucleotide | Purine | C10H15N5O10P2 | RT |
| diphosphate (ADP)² | | Metabolism, | | |
| | | Adenine | | |
| | | containing | | |
| glutathione, reduced | Amino Acid | Glutathione | C10H17N3O6S | MS2; RT |
| (GSH)² | | Metabolism | | |
| glutathione, oxidized | Amino Acid | Glutathione | C19H24N12O10S | MS2; RT |
| (GSSG)² | | Metabolism | | |
| sn-glycero-3- | Other | Glycerophospholipid | C5H14NO6P | MS2 |
| phosphoethanolamine² | | metabolism | | |
| UDP-glucuronate² | Carbohydrate | Nucleotide Sugar | C15H22N2O18P2 | RT |
| N-acetylaspartate | Amino Acid | Alanine and | C6H9NO5 | RT |
| (NAA)² | | Aspartate | | |
| | | Metabolism | | |
| carbamoyl aspartate² | Nucleotide | Pyrimidine | C5H8N2O5 | MS2 |
| | | Metabolism | | |
| glutamate² | Amino Acid | Glutamate | C5H9NO4 | MS2; RT |
| | | Metabolism | | |
| UDP-N- | Carbohydrate | Nucleotide Sugar | C17H27N3O17P2 | RT |
| acetylglucosamine² | | | | |
| methylthioribulose 1- | Other | Other | C6H13O7PS | MS2 |
| phosphate² | | | | |
| lactate² | Carbohydrate | Glycolysis, | C3H6O3 | RT |
| | | Gluconeogenesis, | | |
| | | and Pyruvate | | |
| | | Metabolism | | |
| malate² | Energy | TCA Cycle | C4H6O5 | MS2; RT |
| uridine 5′- | Nucleotide | Pyrimidine | C9H13N2O9P | RT |
| monophosphate | | Metabolism, Uracil | | |
| (UMP)² | | containing | | |
| gamma- | Amino Acid | Glutamate | C4H9NO2 | RT |
| aminobutyrate | | Metabolism | | |
| (GABA)² | | | | |
| 5-oxoproline² | Amino Acid | Glutathione | C5H7NO3 | RT |
| | | Metabolism | | |
| alpha-ketoglutarate² | Energy | TCA Cycle | C5H6O5 | MS2; RT |
| phenylacetylglycine² | Amino Acid; | Phenylalanine and | C10H11NO3 | MS2 |
| | Peptide | Tyrosine | | |
| | | Metabolism | | |
| S-sulfoglutathione² | Other | Other | C13H15N4O6S2 | MS2 |
| histidine² | Amino Acid | Histidine | C6H9N3O2 | MS2; RT |
| | | Metabolism | | |
| asparagine² | Amino Acid | Alanine and | C4H8N2O3 | MS2; RT |
| | | Aspartate | | |
| | | Metabolism | | |
| galactonic acid² | Carbohydrate | Other | C6H12O7 | MS2 |
| beta- | Other | Other | C3H9O6P | MS2 |
| glycerophosphoric | | | | |
| acid² | | | | |
| methylmalonate² | Lipid | Fatty Acid | C4H6O4 | MS2 |
| | | Metabolism | | |
| succinate² | Energy | TCA Cycle | C4H6O4 | RT |
| serine² | Amino Acid | Glycine, Serine | C3H7NO3 | RT |
| | | and Threonine | | |
| | | Metabolism | | |
| 2-hydroxyglutarate² | Lipid | Fatty Acid | C5H8O5 | RT |
| | | Metabolism | | |
| citrate³ | Energy | TCA Cycle | C6H8O7 | RT |
| threonine³ | Amino Acid | Glycine, Serine | C4H9NO3 | MS2 |
| | | and Threonine | | |
| | | Metabolism | | |
| 3- | Other | Other | C16H32O3 | MS2 |
| hydroxyhexadecanoate³ | | | | |
| glutamine³ | Amino Acid | Glutamate | C5H10N2O3 | MS2; RT |
| | | Metabolism | | |
| fructose 1,6- | Carbohydrate | Glycolysis, | C6H14O12P2 | RT |
| biphosphate³ | | Gluconeogenesis, | | |
| | | and Pyruvate | | |
| | | Metabolism | | |
| 4-acetylbutyrate³ | Other | Other | C6H10O3 | MS2 |
| adenosine 5′- | Nucleotide | Purine | C10H16N5O13P3 | RT |
| triphosphate (ATP)³ | | Metabolism, | | |
| | | Adenine | | |
| | | containing | | |
| threonate³ | Other | Other | C4H8O5 | MS2 |
| uridine 5′-triphosphate | Nucleotide | Pyrimidine | C9H15N2O15P3 | RT |
| (UTP)³ | | Metabolism, Uracil | | |
| | | containing | | |
| pentadecanoic acid³ | Other | Other | C15H30O2 | MS2 |
| phenylacetate³ | Amino Acid | Phenylalanine | C8H8O2 | MS2 |
| | | Metabolism | | |
| stearate³ | Lipid | Fatty Acid | C18H36O2 | MS2 |
| | | Metabolism | | |
| phosphoglycolic acid³ | Other | Other | C2H5O6P | MS2 |
| 3-methyl-2- | Amino Acid | Leucine, | C6H10O3 | RT |
| oxovalerate³ | | Isoleucine and | | |
| | | Valine Metabolism | | |
| palmitate³ | Lipid | Long Chain Fatty | C16H32O2 | MS2 |
| | | Acid | | |
| oleate³ | Free Fatty | Free Fatty Acids | C18H34O2 | MS2 |
| | Acids | | | |
| dodecanoate³ | Other | Other | C12H24O2 | MS2 |
| caproate³ | Lipid | Medium Chain | C10H20O2 | MS2 |
| | | Fatty Acid | | |
| 3-hydroxy-3- | Lipid | Mevalonate | C6H10O5 | RT |
| methylglutarate³ | | Metabolism | | |
| N-acetyl-beta-alanine³ | Other | Other | C5H9NO3 | MS2 |

¹Detected in whole cell samples, but not in Mito-IP samples
²Detected in Mito-IP samples, and sgCtrl OMP25HA Mito-IP signal is more than 1.5-fold higher than OMP25Myc Mito-IP signal
³Detected in Mito-IP samples, and sgCtrl OMP25HA Mito-IP signal is less than 1.5-fold higher than OMP25Myc Mito-IP signal

TABLE 1B
Metabolites results

| | DLD1-OMP25HA | DLD1-OMP25HA | DLD1-OMP25HA |
|---|---|---|---|
| | sgCtrl | sgCtrl | sgCtrl |
| Metabolite Name | Whole_cell_rep01 | Whole_cell_rep02 | Whole_cell_rep03 |
| 4-hydroxyproline | 520261 | 394963 | 450843 |
| 6-phosphogluconate | 109277 | 80414 | 87607 |
| aconitate | 662167 | 477271 | 334701 |
| alanine | 1990239 | 1523407 | 1839841 |
| arginine | 1215930 | 1016463 | 1178402 |
| betaine | 204175 | 145964 | 163098 |
| carnitine | 64470 | 58647 | 62159 |
| creatine phosphate | 706076 | 565714 | 637683 |
| creatinine | 268543 | 256326 | 274548 |
| cytidine 5′-triphosphate (CTP) | 98865 | 87527 | 93723 |
| dihydroxyacetone phosphate (DHAP) | 1370428 | 697334 | 1106776 |
| formylpyruvate | 889549 | 699441 | 638919 |
| fructose 6′-phosphate | 242687 | 180794 | 202114 |
| fumarate | 480811 | 380237 | 343411 |
| glucosamine | 238110 | 178065 | 193531 |
| glycerophosphorylcholine (GPC) | 213394 | 164650 | 227894 |
| glycine | 155388 | 135181 | 139322 |
| guanosine 5′-diphosphate (GDP) | 66187 | 56623 | 65720 |
| guanosine 5′-diphosphoglucose | 38575 | 34807 | 40444 |
| guanosine 5′-monophosphate (GMP) | 53850 | 40245 | 48529 |
| guanosine 5′-triphosphate (GTP) | 181160 | 163996 | 193266 |
| hydroxyphenyllactate | 851275 | 632063 | 644073 |
| inosine 5′-monophosphate (IMP) | 27575 | 24191 | 23459 |
| isoleucine | 4760189 | 3886899 | 4542814 |
| leucine | 7803152 | 6519622 | 7574328 |
| lysine | 203044 | 174643 | 216972 |
| methionine | 1061665 | 888276 | 1009835 |
| N6-(delta-isopentenyl)-adenine | 198464 | 159118 | 164924 |
| N-acetylalanine | 244955 | 226732 | 243745 |
| N-acetylglycine | 56501 | 43910 | 46325 |
| N-acetylserine | 8965234 | 6758624 | 4716607 |
| nicotinamide | 41343 | 43384 | 45382 |
| nicotinamide adenine ainucleotide phosphate, | 31005 | 31611 | 33672 |
| reduced (NADPH) | | | |
| nicotinamide adenine dinucleotide phosphate | 18473 | 13118 | 16851 |
| (NADP+) | | | |
| proline | 4160701 | 3272296 | 3701055 |
| ribose 5′-phosphate | 45156 | 28717 | 41338 |
| thymidine 5′-diphospho-alpha-D-glucose | 19443 | 17954 | 19744 |
| thymine | 16845 | 20731 | 22286 |
| tryptophan | 731058 | 586957 | 670131 |
| tyrosine | 4345139 | 3775717 | 4081855 |
| uridine 5′-diphosphate (UDP) | 207545 | 163179 | 167432 |
| valine | 1087024 | 883607 | 1021046 |
| N-acetylglucosamine 6-phosphate | 848332 | 563120 | 628175 |
| N-acetylthreonine | 1686647 | 1440976 | 1405627 |
| galactitol | 704444 | 516643 | 701828 |
| alanylhistidine | 17541 | 19473 | 22327 |
| S-adenosylhomocysteine (SAH) | 3292898 | 2856332 | 2793748 |
| serinylaspartate | 85752 | 87558 | 108542 |
| adenine | 770594 | 676334 | 1161641 |
| folate | 70050 | 99765 | 106276 |
| glycylaspartate | 270276 | 219625 | 236998 |
| glyceraldehyde 3-phosphate | 3446180 | 1747661 | 2799260 |
| 5-methylthioadenosine (MTA) | 415690 | 353580 | 412301 |
| N-acetyltaurine | 1064263 | 1045882 | 1066956 |
| glucose 6′-phosphate | 3264614 | 2539277 | 2771517 |
| nicotinamide adenine dinucleotide, reduced | 255004 | 236621 | 396169 |
| (NADH) | | | |
| phenylalanine | 7146548 | 5851080 | 6758046 |
| pantothenate | 8073292 | 6896530 | 7100157 |
| phosphocholine | 1881749 | 1687362 | 1946133 |
| 2-aminoadipate | 167654 | 138275 | 168677 |
| creatine | 591959 | 458604 | 525249 |
| flavin adenine dinucleotide (FAD) | 121837 | 111300 | 136213 |
| phosphoenolpyruvate (PEP) | 195389 | 164427 | 157991 |
| N-formylmethionine | 325557 | 385732 | 354085 |
| N-acetylmethionine | 480325 | 400496 | 400972 |
| nicotinamide adenine dinucleotide (NAD+) | 9261222 | 8146944 | 9783792 |
| N-acetylglutamate | 996850 | 896379 | 747249 |
| allantoin | 3298767 | 2952184 | 2976496 |
| aspartate | 2969015 | 2250702 | 2469086 |
| taurine | 83933240 | 52786685 | 55774023 |
| adenosine 5′-monophosphate (AMP) | 576714 | 452417 | 538236 |
| adenosine 5′-diphosphate (ADP) | 1151731 | 925909 | 1038965 |
| glutathione, reduced (GSH) | 124292451 | 107547898 | 119074758 |
| glutathione, oxidized (GSSG) | 9869722 | 8635247 | 14339165 |
| sn-glycero-3-phosphoethanolamine | 4496646 | 3704476 | 4570912 |
| UDP-glucuronate | 885012 | 874494 | 873493 |
| N-acetylaspartate (NAA) | 14323397 | 12157294 | 10250580 |
| carbamoyl aspartate | 4151110 | 3945862 | 3592438 |
| glutamate | 145611252 | 125951469 | 137016779 |
| UDP-N-acetylglucosamine | 7200966 | 6285659 | 7273876 |
| methylthioribulose 1-phosphate | 27043571 | 20586674 | 22559163 |
| lactate | 81796343 | 80473218 | 87572717 |
| malate | 10928845 | 8615884 | 7231583 |
| uridine 5′-monophosphate (UMP) | 419841 | 372112 | 295745 |
| gamma-aminobutyrate (GABA) | 4090349 | 3518803 | 3892729 |
| 5-oxoproline | 13218064 | 12329959 | 11429855 |
| alpha-ketoglutarate | 6832510 | 5791191 | 5495124 |
| phenylacetylglycine | 1845213 | 1775939 | 1819968 |
| S-sulfoglutathione | 186671 | 171322 | 180074 |
| histidine | 15950901 | 13114852 | 10969698 |
| asparagine | 2764045 | 1997220 | 1987282 |
| galactonic acid | 3173840 | 2336362 | 2118717 |
| beta-glycerophosphoric acid | 1064812 | 733499 | 725977 |
| methylmalonate | 646704 | 537380 | 574678 |
| succinate | 350207 | 304264 | 318665 |
| serine | 1978329 | 1506569 | 1499351 |
| 2-hydroxyglutarate | 3451454 | 2965856 | 2435193 |
| citrate | 13048797 | 10385301 | 8224134 |
| threonine | 14547296 | 12565854 | 13706882 |
| 3-hydroxyhexadecanoate | 165784 | 229355 | 326158 |
| glutamine | 63543190 | 69223709 | 81580307 |
| fructose 1,6-biphosphate | 825110 | 743444 | 928746 |
| 4-acetylbutyrate | 6109906 | 7192466 | 8206674 |
| adenosine 5′-triphosphate (ATP) | 2458947 | 2224975 | 2322844 |
| threonate | 6309708 | 5090303 | 3588894 |
| uridine 5′-triphosphate (UTP) | 2559894 | 2395837 | 2506549 |
| pentadecanoic acid | 443272 | 232835 | 318229 |
| phenylacetate | 158955 | 121447 | 137083 |
| stearate | 17433339 | 14538319 | 13841315 |
| phosphoglycolic acid | 39426 | 48688 | 30501 |
| 3-methyl-2-oxovalerate | 2049788 | 2513980 | 2870670 |
| palmitate | 31520159 | 27579252 | 26214403 |
| oleate | 609969 | 452464 | 449134 |
| dodecanoate | 541092 | 404585 | 412289 |
| caproate | 158049 | 162863 | 135301 |
| 3-hydroxy-3-methylglutarate | 236492 | 205860 | 155901 |
| N-acetyl-beta-alanine | 1221435 | 1030480 | 891197 |

TABLE 1C
Metabolites results

| | DLD1- | DLD1- | DLD1- | DLD1- | DLD1- | DLD1- |
|---|---|---|---|---|---|---|
| | OMP25HA | OMP25HA | OMP25HA | OMP25HA | OMP25HA | OMP25HA |
| | sgNADK2-1 | sgNADK2-1 | sgNADK2-1 | sgNADK2-2 | sgNADK2-2 | sgNADK2-2 |
| | Whole_cell_{—} | Whole_cell_{—} | Whole_cell_{—} | Whole_cell_{—} | Whole_cell_{—} | Whole_cell_{—} |
| Metabolite Name | rep01 | rep02 | rep03 | rep01 | rep02 | rep03 |
| 4-hydroxyproline | 507156 | 449374 | 532479 | 457044 | 467175 | 479002 |
| 6-phosphogluconate | 99053 | 96089 | 94716 | 84488 | 105282 | 84226 |
| aconitate | 81496 | 60618 | 83630 | 418055 | 328314 | 490949 |
| alanine | 1791643 | 1754401 | 2059569 | 1595846 | 1647509 | 1696676 |
| arginine | 1069771 | 1179167 | 1212495 | 899829 | 1047368 | 766713 |
| betaine | 155624 | 146764 | 198116 | 142593 | 176471 | 159019 |
| carnitine | 64093 | 69680 | 64879 | 60766 | 74199 | 65886 |
| creatine phosphate | 573078 | 619525 | 655060 | 545460 | 556332 | 555426 |
| creatinine | 267829 | 257396 | 292168 | 226354 | 270078 | 243136 |
| cytidine 5′-triphosphate | 77218 | 92749 | 85319 | 89330 | 105227 | 60528 |
| (CTP) | | | | | | |
| dihydroxyacetone phosphate | 1483416 | 1429390 | 1716439 | 942541 | 1232703 | 996644 |
| (DHAP) | | | | | | |
| formylpyruvate | 778655 | 721101 | 915302 | 659756 | 762314 | 700152 |
| fructose 6′-phosphate | 154638 | 150298 | 175462 | 170873 | 201181 | 180561 |
| fumarate | 567684 | 484040 | 572405 | 418478 | 389849 | 488625 |
| glucosamine | 181763 | 161878 | 219715 | 162321 | 204990 | 183860 |
| glycerophosphorylcholine | 166268 | 170556 | 174555 | 165021 | 181380 | 184173 |
| (GPC) | | | | | | |
| glycine | 158289 | 140611 | 163653 | 148904 | 128950 | 149840 |
| guanosine 5′-diphosphate | 60064 | 58203 | 80826 | 53867 | 66835 | 60936 |
| (GDP) | | | | | | |
| guanosine 5′- | 32331 | 39426 | 38344 | 39329 | 45289 | 31511 |
| diphosphoglucose | | | | | | |
| guanosine 5′-monophosphate | 63074 | 53310 | 65103 | 57970 | 51265 | 51999 |
| (GMP) | | | | | | |
| guanosine 5′-triphosphate | 150650 | 141179 | 183725 | 168855 | 190507 | 158260 |
| (GTP) | | | | | | |
| hydroxyphenyllactate | 867334 | 829579 | 912438 | 711625 | 944631 | 846861 |
| inosine 5′-monophosphate | 36494 | 41413 | 41682 | 41939 | 45496 | 41371 |
| (IMP) | | | | | | |
| isoleucine | 4472514 | 4261285 | 4806873 | 3761406 | 4301166 | 4259590 |
| leucine | 7637565 | 7258695 | 7942100 | 6467313 | 7112905 | 7371831 |
| lysine | 81821 | 216942 | 216658 | 157360 | 187764 | 137459 |
| methionine | 1059486 | 1002117 | 1035181 | 871355 | 935238 | 966084 |
| N6-(delta-isopentenyl)- | 247141 | 230378 | 259213 | 188310 | 200713 | 219300 |
| adenine | | | | | | |
| N-acetylalanine | 273147 | 263679 | 287211 | 241184 | 248777 | 265444 |
| N-acetylglycine | 55880 | 48949 | 46952 | 49249 | 49300 | 57019 |
| N-acetylserine | 9653395 | 7034818 | 9220865 | 6444772 | 7467278 | 7136644 |
| nicotinamide | 50352 | 46851 | 55434 | 33061 | 38680 | 39976 |
| nicotinamide adenine | 28536 | 31627 | 44253 | 20179 | 28518 | 25273 |
| ainucleotide phosphate, | | | | | | |
| reduced (NADPH) | | | | | | |
| nicotinamide adenine | 17933 | 14414 | 21413 | 10649 | 14712 | 15081 |
| dinucleotide phosphate | | | | | | |
| (NADP+) | | | | | | |
| proline | 3771102 | 3619274 | 4412673 | 3413457 | 3837210 | 3680484 |
| ribose 5′-phosphate | 40910 | 45821 | 61290 | 37972 | 47420 | 41422 |
| thymidine 5′-diphospho- | 13810 | 15269 | 17930 | 14818 | 18175 | 17376 |
| alpha-D-glucose | | | | | | |
| thymine | 17576 | 18639 | 19038 | 12597 | 16125 | 14897 |
| tryptophan | 706919 | 659837 | 739986 | 593195 | 626519 | 687657 |
| tyrosine | 4491078 | 4275635 | 4459365 | 3598243 | 2999427 | 3604730 |
| uridine 5′-diphosphate (UDP) | 149741 | 168585 | 164383 | 198714 | 215827 | 189296 |
| valine | 1027106 | 964958 | 1142580 | 895703 | 964131 | 966032 |
| N-acetylglucosamine 6- | 525962 | 549362 | 644299 | 473309 | 579423 | 520943 |
| phosphate | | | | | | |
| N-acetylthreonine | 1669011 | 1470496 | 1486451 | 1487021 | 1498581 | 1737270 |
| galactitol | 721269 | 683340 | 1031609 | 633331 | 856520 | 835474 |
| alanylhistidine | 21878 | 23539 | 20828 | 16168 | 17818 | 18385 |
| S-adenosylhomocysteine | 2423361 | 2587703 | 2299041 | 2756301 | 2988972 | 3005734 |
| (SAH) | | | | | | |
| serinylaspartate | 120994 | 118953 | 94546 | 94067 | 97744 | 140998 |
| adenine | 1396979 | 1329498 | 831362 | 598934 | 588374 | 701669 |
| folate | 105946 | 104266 | 88245 | 87305 | 63927 | 103163 |
| glycylaspartate | 276580 | 280939 | 308340 | 240816 | 245906 | 247046 |
| glyceraldehyde 3-phosphate | 3780432 | 3639698 | 4307527 | 2356617 | 3093124 | 2521792 |
| 5-methylthioadenosine | 429781 | 494599 | 423656 | 316919 | 313324 | 372346 |
| (MTA) | | | | | | |
| N-acetyltaurine | 1283076 | 1210943 | 1138898 | 1187267 | 1086274 | 1446679 |
| glucose 6′-phosphate | 2101877 | 2037131 | 2356642 | 2373648 | 2638855 | 2497724 |
| nicotinamide adenine | 1297824 | 1243566 | 1254702 | 280024 | 356755 | 421132 |
| dinucleotide, reduced | | | | | | |
| (NADH) | | | | | | |
| phenylalanine | 7183560 | 6495860 | 7098821 | 5963980 | 6278026 | 6889157 |
| pantothenate | 7468885 | 7196151 | 7602618 | 6763255 | 8062290 | 7394751 |
| phosphocholine | 1669771 | 1789380 | 1778143 | 1634181 | 1780323 | 1668892 |
| 2-aminoadipate | 137782 | 144304 | 155661 | 125546 | 134299 | 133926 |
| creatine | 492822 | 509909 | 604936 | 454239 | 541788 | 498075 |
| flavin adenine dinucleotide | 84652 | 119038 | 106744 | 128021 | 141636 | 119558 |
| (FAD) | | | | | | |
| phosphoenolpyruvate (PEP) | 195748 | 183791 | 226688 | 146311 | 178293 | 176649 |
| N-formylmethionine | 364186 | 345801 | 330077 | 291502 | 328963 | 298397 |
| N-acetylmethionine | 524834 | 498737 | 455184 | 441504 | 452506 | 505747 |
| nicotinamide adenine | 7217894 | 6883560 | 7593185 | 9153066 | 9720699 | 9380397 |
| dinucleotide (NAD+) | | | | | | |
| N-acetylglutamate | 1409651 | 1222671 | 1366684 | 1171559 | 1342788 | 1484319 |
| allantoin | 3332039 | 3383651 | 3467652 | 3195460 | 3332349 | 3249164 |
| aspartate | 1756042 | 1527560 | 1709488 | 1630770 | 1809987 | 1819134 |
| taurine | 58659965 | 55858681 | 60594324 | 54104556 | 76401410 | 55581208 |
| adenosine 5′-monophosphate | 506262 | 549275 | 550004 | 511751 | 511701 | 526076 |
| (AMP) | | | | | | |
| adenosine 5′-diphosphate | 1219836 | 1076788 | 1469178 | 891698 | 1067253 | 1003291 |
| (ADP) | | | | | | |
| glutathione, reduced (GSH) | 111746488 | 127750916 | 123977767 | 112563173 | 123308168 | 97337061 |
| glutathione, oxidized (GSSG) | 10910308 | 11606828 | 9375218 | 9652656 | 9402637 | 15845674 |
| sn-glycero-3- | 3983197 | 3994969 | 4251753 | 3564030 | 3824106 | 4236126 |
| phosphoethanolamine | | | | | | |
| UDP-glucuronate | 935312 | 926045 | 896416 | 1132223 | 1303620 | 776085 |
| N-acetylaspartate (NAA) | 13281164 | 11433009 | 12856127 | 11446686 | 13125253 | 13072893 |
| carbamoyl aspartate | 4869582 | 4508220 | 4500303 | 4204360 | 4348690 | 4957311 |
| glutamate | 142071277 | 143723577 | 148812917 | 131146159 | 144081050 | 145844987 |
| UDP-N-acetylglucosamine | 5677822 | 6425460 | 6036325 | 5476359 | 5673795 | 5779323 |
| methylthioribulose 1- | 22221062 | 23697212 | 27368016 | 20013038 | 24740250 | 21233864 |
| phosphate | | | | | | |
| lactate | 99050739 | 103396949 | 116817271 | 83735203 | 95145326 | 93410483 |
| malate | 9501227 | 8642854 | 11242287 | 7919098 | 9034587 | 8281193 |
| uridine 5′-monophosphate | 321552 | 359308 | 329122 | 369602 | 417677 | 402263 |
| (UMP) | | | | | | |
| gamma-aminobutyrate | 3987097 | 4057753 | 4218698 | 3682410 | 3978317 | 4101502 |
| (GABA) | | | | | | |
| 5-oxoproline | 15754716 | 14744655 | 17743818 | 12248150 | 13884035 | 13823005 |
| alpha-ketoglutarate | 8951552 | 8539236 | 9657853 | 7174227 | 8256253 | 7227204 |
| phenylacetylglycine | 1914223 | 1842175 | 1898543 | 1530965 | 1881080 | 1733654 |
| S-sulfoglutathione | 274981 | 244155 | 231606 | 225106 | 273396 | 255353 |
| histidine | 12042662 | 10828819 | 16146558 | 13885827 | 13681787 | 11420299 |
| asparagine | 1961797 | 1593532 | 1983913 | 1817254 | 1914115 | 1844948 |
| galactonic acid | 2983436 | 2708030 | 3546559 | 2761948 | 3355628 | 3021179 |
| beta-glycerophosphoric acid | 876187 | 936231 | 1222814 | 829282 | 1098870 | 852326 |
| methylmalonate | 908707 | 865996 | 1007015 | 851241 | 1024665 | 1193666 |
| succinate | 248738 | 950616 | 208246 | 510675 | 482429 | 571738 |
| serine | 1994443 | 1586543 | 1890703 | 1828461 | 1866309 | 2004244 |
| 2-hydroxyglutarate | 4862720 | 4387267 | 4964997 | 4217987 | 4857848 | 5066364 |
| citrate | 5905883 | 4688074 | 5394791 | 10618744 | 8631857 | 11316473 |
| threonine | 15215895 | 13613634 | 14946896 | 14221656 | 12227246 | 14698731 |
| 3-hydroxyhexadecanoate | 218773 | 888777 | 727028 | 59845 | 52353 | 104726 |
| glutamine | 89161549 | 84481040 | 64840721 | 82062360 | 61871289 | 90038147 |
| fructose 1,6-biphosphate | 990396 | 1093435 | 984967 | 835965 | 907106 | 700530 |
| 4-acetylbutyrate | 10620692 | 10766693 | 11811826 | 7954825 | 7948029 | 9023914 |
| adenosine 5′-triphosphate | 2082891 | 1854182 | 2449124 | 1996814 | 2295770 | 2198862 |
| (ATP) | | | | | | |
| threonate | 6889498 | 5503497 | 7620884 | 5341221 | 5615302 | 5242629 |
| uridine 5′-triphosphate (UTP) | 2378772 | 2585673 | 2450838 | 2920714 | 3444592 | 2053733 |
| pentadecanoic acid | 301200 | 414048 | 303072 | 206352 | 287981 | 212354 |
| phenylacetate | 167198 | 143316 | 153049 | 130674 | 110333 | 143493 |
| stearate | 16513954 | 14907565 | 13697244 | 13353007 | 15943904 | 16071248 |
| phosphoglycolic acid | 42353 | 38457 | 37411 | 59217 | 61987 | 69116 |
| 3-methyl-2-oxovalerate | 3715334 | 3733035 | 4128294 | 2732810 | 2823142 | 3206971 |
| palmitate | 27684576 | 27818767 | 25221182 | 25074380 | 29317005 | 30933293 |
| oleate | 432915 | 564318 | 608016 | 343454 | 370018 | 996789 |
| dodecanoate | 552648 | 469531 | 394553 | 386324 | 365381 | 407674 |
| caproate | 169403 | 152069 | 144414 | 117435 | 132034 | 148404 |
| 3-hydroxy-3-methylglutarate | 255008 | 215115 | 257279 | 172958 | 217678 | 231572 |
| N-acetyl-beta-alanine | 1128814 | 973388 | 1091252 | 968354 | 1101600 | 1088820 |

TABLE 1D
Metabolites results

| | DLD1- | DLD1- | DLD1- | | | |
|---|---|---|---|---|---|---|
| | OMP25HA | OMP25HA | OMP25HA | | | |
| | sgCtrl + | sgCtrl + | sgCtrl + | DLD1- | DLD1- | DLD1- |
| | Piericidin | Piericidin | Piericidin | OMP25Myc | OMP25Myc | OMP25Myc |
| | Whole_{—} | Whole_{—} | Whole_{—} | Whole_{—} | Whole_{—} | Whole_{—} |
| Metabolite Name | cell_rep01 | cell_rep02 | cell_rep03 | cell_rep01 | cell_rep02 | cell_rep03 |
| 4-hydroxyproline | 522424 | 441928 | 551397 | 334009 | 339052 | 413777 |
| 6-phosphogluconate | 65481 | 57701 | 58966 | 50191 | 58623 | 51065 |
| aconitate | 346582 | 453318 | 406006 | 455873 | 272412 | 358020 |
| alanine | 1663860 | 1344230 | 1765231 | 1064197 | 1044796 | 1273614 |
| arginine | 1086834 | 998509 | 1348136 | 632120 | 787456 | 994595 |
| betaine | 161649 | 112014 | 159770 | 83663 | 112743 | 136760 |
| carnitine | 43283 | 41086 | 45131 | 39003 | 57322 | 61446 |
| creatine phosphate | 394087 | 351223 | 410441 | 402872 | 506738 | 535821 |
| creatinine | 277361 | 240499 | 323168 | 201663 | 230706 | 289856 |
| cytidine 5′-triphosphate (CTP) | 81066 | 75474 | 92586 | 79001 | 95166 | 87345 |
| dihydroxyacetone phosphate | 1214605 | 1228567 | 1670800 | 534599 | 772455 | 1095825 |
| (DHAP) | | | | | | |
| formylpyruvate | 1414077 | 902945 | 1059691 | 609412 | 507783 | 582764 |
| fructose 6′-phosphate | 57127 | 49316 | 50145 | 79369 | 97386 | 108343 |
| fumarate | 357661 | 412548 | 377283 | 317842 | 273078 | 319402 |
| glucosamine | 164826 | 119694 | 178113 | 99305 | 134495 | 151506 |
| glycerophosphorylcholine (GPC) | 210330 | 183098 | 225230 | 165563 | 269416 | 280213 |
| glycine | 146868 | 137614 | 144892 | 94864 | 97029 | 120445 |
| guanosine 5′-diphosphate (GDP) | 69660 | 69853 | 38046 | 51196 | 64968 | 64935 |
| guanosine 5′-diphosphoglucose | 33192 | 34147 | 38993 | 25362 | 35166 | 31290 |
| guanosine 5′-monophosphate | 44633 | 51573 | 51777 | 35788 | 45522 | 47540 |
| (GMP) | | | | | | |
| guanosine 5′-triphosphate (GTP) | 171526 | 214847 | 133658 | 166521 | 197014 | 166366 |
| hydroxyphenyllactate | 1478092 | 1166698 | 1355443 | 410141 | 509138 | 635295 |
| inosine 5′-monophosphate (IMP) | 22897 | 17939 | 22222 | 15970 | 20569 | 19544 |
| isoleucine | 4513788 | 3853543 | 5003963 | 2357913 | 3038333 | 3541351 |
| leucine | 7669096 | 6479727 | 8479119 | 4189435 | 5287942 | 6109202 |
| lysine | 195097 | 149255 | 233140 | 116756 | 155296 | 190570 |
| methionine | 1041135 | 894784 | 1078995 | 586018 | 722819 | 849258 |
| N6-(delta-isopentenyl)-adenine | 293077 | 229998 | 281738 | 62118 | 67047 | 75866 |
| N-acetylalanine | 238161 | 214277 | 230562 | 146986 | 170111 | 166210 |
| N-acetylglycine | 43846 | 40358 | 41154 | 33220 | 38637 | 46811 |
| N-acetylserine | 5896001 | 5058414 | 6358391 | 5099268 | 3377119 | 4138331 |
| nicotinamide | 39081 | 33023 | 41574 | 22660 | 28852 | 36367 |
| nicotinamide adenine ainucleotide | 30643 | 34194 | 16379 | 26343 | 39042 | 34988 |
| phosphate, reduced (NADPH) | | | | | | |
| nicotinamide adenine dinucleotide | 15380 | 19291 | 12614 | 12983 | 18307 | 17646 |
| phosphate (NADP+) | | | | | | |
| proline | 4018244 | 3303744 | 4136919 | 1873767 | 2213854 | 2541367 |
| ribose 5′-phosphate | 30487 | 39389 | 44236 | 16807 | 27337 | 29839 |
| thymidine 5′-diphospho-alpha-D- | 14725 | 13909 | 14870 | 11763 | 14353 | 14498 |
| glucose | | | | | | |
| thymine | 24681 | 21055 | 27340 | 12155 | 18836 | 26937 |
| tryptophan | 747292 | 595893 | 756562 | 325293 | 454917 | 543601 |
| tyrosine | 3847092 | 3576189 | 3918008 | 1802085 | 2565007 | 2715738 |
| uridine 5′-diphosphate (UDP) | 175584 | 155688 | 210038 | 146735 | 188868 | 183505 |
| valine | 1041408 | 903609 | 1081229 | 544097 | 678668 | 820137 |
| N-acetylglucosamine 6-phosphate | 428986 | 380372 | 454385 | 335005 | 469029 | 523518 |
| N-acetylthreonine | 1380430 | 1226851 | 1295989 | 1001994 | 1149269 | 1274769 |
| galactitol | 918739 | 708477 | 1199966 | 619905 | 589335 | 982442 |
| alanylhistidine | 22199 | 41823 | 30446 | 18876 | 25124 | 33533 |
| S-adenosylhomocysteine (SAH) | 3954592 | 1812665 | 2746769 | 1641526 | 2282184 | 2298478 |
| serinylaspartate | 80143 | 82279 | 87822 | 91328 | 82142 | 80500 |
| adenine | 481304 | 768689 | 708555 | 389664 | 474855 | 514344 |
| folate | 99228 | 128177 | 134416 | 103222 | 118315 | 162690 |
| glycylaspartate | 205091 | 201360 | 226481 | 139540 | 166196 | 177268 |
| glyceraldehyde 3-phosphate | 3045295 | 3098709 | 4229751 | 1343480 | 1969972 | 2773036 |
| 5-methylthioadenosine (MTA) | 257749 | 400467 | 367942 | 159088 | 237857 | 266506 |
| N-acetyltaurine | 1110210 | 1084752 | 1148548 | 569289 | 791397 | 911107 |
| glucose 6′-phosphate | 755029 | 621609 | 531377 | 918256 | 1069266 | 1167856 |
| nicotinamide adenine dinucleotide, | 455322 | 465048 | 452884 | 431983 | 787568 | 524756 |
| reduced (NADH) | | | | | | |
| phenylalanine | 6841254 | 5972052 | 7346184 | 3603970 | 4450893 | 5201680 |
| pantothenate | 8937050 | 7961239 | 9124782 | 5393575 | 7648101 | 8344896 |
| phosphocholine | 1734241 | 1590770 | 1746457 | 1116555 | 1698220 | 1634784 |
| 2-aminoadipate | 146679 | 129247 | 159230 | 93562 | 112385 | 123752 |
| creatine | 549841 | 451406 | 565679 | 292813 | 359013 | 408053 |
| flavin adenine dinucleotide (FAD) | 115513 | 108031 | 137050 | 77728 | 116378 | 104238 |
| phosphoenolpyruvate (PEP) | 217348 | 206448 | 247608 | 155300 | 147967 | 165732 |
| N-formylmethionine | 320459 | 98350 | 410191 | 154738 | 203533 | 289903 |
| N-acetylmethionine | 462515 | 426811 | 428397 | 228128 | 279360 | 312239 |
| nicotinamide adenine dinucleotide | 7875605 | 8688241 | 9053967 | 4340653 | 7403520 | 6757326 |
| (NAD+) | | | | | | |
| N-acetylglutamate | 987898 | 1549149 | 871723 | 964925 | 879034 | 983544 |
| allantoin | 3703951 | 3225148 | 4031072 | 2272309 | 3434783 | 4050278 |
| aspartate | 1717174 | 1947525 | 1819639 | 2793950 | 3014462 | 3369123 |
| taurine | 79062900 | 54111662 | 81152564 | 43920947 | 51436211 | 55253503 |
| adenosine 5′-monophosphate | 646021 | 715531 | 648428 | 321171 | 502169 | 473084 |
| (AMP) | | | | | | |
| adenosine 5′-diphosphate (ADP) | 1129524 | 1189412 | 1083190 | 844515 | 1158951 | 1106578 |
| glutathione, reduced (GSH) | 91764096 | 3859852 | 117518132 | 28838409 | 39292826 | 44129612 |
| glutathione, oxidized (GSSG) | 10455192 | 21668761 | 8823872 | 11846450 | 18867838 | 20256384 |
| sn-glycero-3- | 3947102 | 4703023 | 3861169 | 3114652 | 4183629 | 4478212 |
| phosphoethanolamine | | | | | | |
| UDP-glucuronate | 1027925 | 858275 | 1243921 | 1136028 | 1194600 | 1220285 |
| N-acetylaspartate (NAA) | 12095956 | 11050203 | 12351863 | 11459901 | 10089471 | 11591131 |
| carbamoyl aspartate | 3763133 | 3469742 | 3815379 | 4287116 | 3940471 | 4321229 |
| glutamate | 126622339 | 129663305 | 129294077 | 109129441 | 126305488 | 134897061 |
| UDP-N-acetylglucosamine | 4639411 | 4238311 | 4998970 | 3171852 | 4960395 | 4732270 |
| methylthioribulose 1-phosphate | 21298086 | 18010540 | 22620900 | 11222296 | 15770199 | 17704682 |
| lactate | 105521436 | 97172905 | 114401680 | 49713905 | 54902329 | 63206064 |
| malate | 12653099 | 10480180 | 12542946 | 7594310 | 5868569 | 6786254 |
| uridine 5′-monophosphate (UMP) | 631970 | 347896 | 460624 | 303328 | 329680 | 365083 |
| gamma-aminobutyrate (GABA) | 3496195 | 3622488 | 3524195 | 2957088 | 3519439 | 3751394 |
| 5-oxoproline | 16393034 | 14068076 | 17132083 | 6873255 | 7325637 | 9444088 |
| alpha-ketoglutarate | 8532039 | 4145995 | 10151697 | 3813285 | 3883463 | 4616286 |
| phenylacetylglycine | 1781914 | 1641866 | 2033368 | 1037937 | 1422761 | 1826573 |
| S-sulfoglutathione | 360443 | 671375 | 291298 | 158314 | 199447 | 244925 |
| histidine | 15027640 | 12343272 | 17476456 | 6212294 | 9510469 | 12608988 |
| asparagine | 2068551 | 1580346 | 2240202 | 1324962 | 1344073 | 1739126 |
| galactonic acid | 2240293 | 1677985 | 2390572 | 1815985 | 1797234 | 2443097 |
| beta-glycerophosphoric acid | 850446 | 705915 | 880253 | 438000 | 501277 | 570012 |
| methylmalonate | 461951 | 1683788 | 394694 | 680688 | 701208 | 807173 |
| succinate | 468012 | 552589 | 622074 | 360665 | 387672 | 439049 |
| serine | 1972729 | 1697021 | 2115421 | 1569904 | 1341804 | 1660898 |
| 2-hydroxyglutarate | 3675897 | 3670344 | 3586833 | 2993328 | 2196013 | 2550572 |
| citrate | 9028971 | 10970194 | 9594217 | 10834126 | 8250767 | 9944184 |
| threonine | 13508533 | 12463455 | 14159756 | 9542978 | 9811544 | 11519714 |
| 3-hydroxyhexadecanoate | 45705 | 143119 | 470888 | 51886 | 175757 | 835411 |
| glutamine | 58464656 | 69652329 | 62470821 | 52321636 | 62342257 | 51695774 |
| fructose 1,6-biphosphate | 1321982 | 1216851 | 1680580 | 908907 | 1077582 | 1153131 |
| 4-acetylbutyrate | 10363291 | 8533694 | 12130178 | 2805146 | 3724533 | 4581922 |
| adenosine 5′-triphosphate (ATP) | 2352445 | 2990040 | 2388618 | 1843829 | 2297651 | 2099754 |
| threonate | 4663562 | 3938483 | 4644285 | 3288440 | 2567934 | 3270392 |
| uridine 5′-triphosphate (UTP) | 3135652 | 2793092 | 3584193 | 2406549 | 2655071 | 2527463 |
| pentadecanoic acid | 302982 | 182808 | 229103 | 160446 | 244255 | 322747 |
| phenylacetate | 87967 | 87598 | 109383 | 51231 | 63564 | 65391 |
| stearate | 10555350 | 11041062 | 14072247 | 7996177 | 12945930 | 16021706 |
| phosphoglycolic acid | 107615 | 52600 | 35470 | 44618 | 28623 | 30976 |
| 3-methyl-2-oxovalerate | 3696272 | 2980891 | 4249362 | 943896 | 1292429 | 1673113 |
| palmitate | 20015231 | 20994808 | 24605738 | 15816223 | 23530788 | 28940672 |
| oleate | 460639 | 248285 | 328228 | 503902 | 492233 | 708790 |
| dodecanoate | 303807 | 282352 | 360172 | 187527 | 283973 | 290037 |
| caproate | 114886 | 109032 | 115791 | 73353 | 98509 | 103387 |
| 3-hydroxy-3-methylglutarate | 171203 | 140919 | 19965 | 112921 | 84853 | 111975 |
| N-acetyl-beta-alanine | 1022765 | 956905 | 1036571 | 942298 | 867699 | 1002045 |

TABLE 1E
Metabolites results

| | DLD1- | DLD1- | DLD1- | DLD1- | DLD1- | DLD1- |
|---|---|---|---|---|---|---|
| | OMP25HA | OMP25HA | OMP25HA | OMP25HA | OMP25HA | OMP25HA |
| | sgCtrl | sgCtrl | sgCtrl | sgNADK2-1 | sgNADK2-1 | sgNADK2-1 |
| | MitoIP_{—} | MitoIP_{—} | MitoIP_{—} | MitoIP_{—} | MitoIP_{—} | MitoIP_{—} |
| Metabolite Name | rep01 | rep02 | rep03 | rep01 | rep02 | rep03 |
| 4-hydroxyproline | not | not | not | not | not | not |
| | detected | detected | detected | detected | detected | detected |
| 6-phosphogluconate | not | not | not | not | not | not |
| | detected | detected | detected | detected | detected | detected |
| aconitate | not | not | not | not | not | not |
| | detected | detected | detected | detected | detected | detected |
| alanine | not | not | not | not | not | not |
| | detected | detected | detected | detected | detected | detected |
| arginine | not | not | not | not | not | not |
| | detected | detected | detected | detected | detected | detected |
| betaine | not | not | not | not | not | not |
| | detected | detected | detected | detected | detected | detected |
| carnitine | not | not | not | not | not | not |
| | detected | detected | detected | detected | detected | detected |
| creatine phosphate | not | not | not | not | not | not |
| | detected | detected | detected | detected | detected | detected |
| creatinine | not | not | not | not | not | not |
| | detected | detected | detected | detected | detected | detected |
| cytidine 5′-triphosphate (CTP) | not | not | not | not | not | not |
| | detected | detected | detected | detected | detected | detected |
| dihydroxyacetone phosphate (DHAP) | not | not | not | not | not | not |
| | detected | detected | detected | detected | detected | detected |
| formylpyruvate | not | not | not | not | not | not |
| | detected | detected | detected | detected | detected | detected |
| fructose 6′-phosphate | not | not | not | not | not | not |
| | detected | detected | detected | detected | detected | detected |
| fumarate | not | not | not | not | not | not |
| | detected | detected | detected | detected | detected | detected |
| glucosamine | not | not | not | not | not | not |
| | detected | detected | detected | detected | detected | detected |
| glycerophosphorylcholine (GPC) | not | not | not | not | not | not |
| | detected | detected | detected | detected | detected | detected |
| glycine | not | not | not | not | not | not |
| | detected | detected | detected | detected | detected | detected |
| guanosine 5′-diphosphate (GDP) | not | not | not | not | not | not |
| | detected | detected | detected | detected | detected | detected |
| guanosine 5′-diphosphoglucose | not | not | not | not | not | not |
| | detected | detected | detected | detected | detected | detected |
| guanosine 5′-monophosphate (GMP) | not | not | not | not | not | not |
| | detected | detected | detected | detected | detected | detected |
| guanosine 5′-triphosphate (GTP) | not | not | not | not | not | not |
| | detected | detected | detected | detected | detected | detected |
| hydroxyphenyllactate | not | not | not | not | not | not |
| | detected | detected | detected | detected | detected | detected |
| inosine 5′-monophosphate (IMP) | not | not | not | not | not | not |
| | detected | detected | detected | detected | detected | detected |
| isoleucine | not | not | not | not | not | not |
| | detected | detected | detected | detected | detected | detected |
| leucine | not | not | not | not | not | not |
| | detected | detected | detected | detected | detected | detected |
| lysine | not | not | not | not | not | not |
| | detected | detected | detected | detected | detected | detected |
| methionine | not | not | not | not | not | not |
| | detected | detected | detected | detected | detected | detected |
| N6-(delta-isopentenyl)-adenine | not | not | not | not | not | not |
| | detected | detected | detected | detected | detected | detected |
| N-acetylalanine | not | not | not | not | not | not |
| | detected | detected | detected | detected | detected | detected |
| N-acetylglycine | not | not | not | not | not | not |
| | detected | detected | detected | detected | detected | detected |
| N-acetylserine | not | not | not | not | not | not |
| | detected | detected | detected | detected | detected | detected |
| nicotinamide | not | not | not | not | not | not |
| | detected | detected | detected | detected | detected | detected |
| nicotinamide adenine ainucleotide | not | not | not | not | not | not |
| phosphate, reduced (NADPH) | detected | detected | detected | detected | detected | detected |
| nicotinamide adenine dinucleotide | not | not | not | not | not | not |
| phosphate (NADP+) | detected | detected | detected | detected | detected | detected |
| proline | not | not | not | not | not | not |
| | detected | detected | detected | detected | detected | detected |
| ribose 5′-phosphate | not | not | not | not | not | not |
| | detected | detected | detected | detected | detected | detected |
| thymidine 5′-diphospho-alpha-D- | not | not | not | not | not | not |
| glucose | detected | detected | detected | detected | detected | detected |
| thymine | not | not | not | not | not | not |
| | detected | detected | detected | detected | detected | detected |
| tryptophan | not | not | not | not | not | not |
| | detected | detected | detected | detected | detected | detected |
| tyrosine | not | not | not | not | not | not |
| | detected | detected | detected | detected | detected | detected |
| uridine 5′-diphosphate (UDP) | not | not | not | not | not | not |
| | detected | detected | detected | detected | detected | detected |
| valine | not | not | not | not | not | not |
| | detected | detected | detected | detected | detected | detected |
| N-acetylglucosamine 6-phosphate | not | not | not | not | not | not |
| | detected | detected | detected | detected | detected | detected |
| N-acetylthreonine | not | not | not | not | not | not |
| | detected | detected | detected | detected | detected | detected |
| galactitol | not | not | not | not | not | not |
| | detected | detected | detected | detected | detected | detected |
| alanylhistidine | not | not | not | not | not | not |
| | detected | detected | detected | detected | detected | detected |
| S-adenosylhomocysteine (SAH) | not | not | not | not | not | not |
| | detected | detected | detected | detected | detected | detected |
| serinylaspartate | not | not | not | not | not | not |
| | detected | detected | detected | detected | detected | detected |
| adenine | not | not | not | not | not | not |
| | detected | detected | detected | detected | detected | detected |
| folate | not | not | not | not | not | not |
| | detected | detected | detected | detected | detected | detected |
| glycylaspartate | not | not | not | not | not | not |
| | detected | detected | detected | detected | detected | detected |
| glyceraldehyde 3-phosphate | not | not | not | not | not | not |
| | detected | detected | detected | detected | detected | detected |
| 5-methylthioadenosine (MTA) | not | not | not | not | not | not |
| | detected | detected | detected | detected | detected | detected |
| N-acetyltaurine | not | not | not | not | not | not |
| | detected | detected | detected | detected | detected | detected |
| glucose 6′-phosphate | not | not | not | not | not | not |
| | detected | detected | detected | detected | detected | detected |
| nicotinamide adenine dinucleotide, | 357192 | 527789 | 555833 | 361493 | 443903 | 583358 |
| reduced (NADH) | | | | | | |
| phenylalanine | 42482 | 39614 | 53765 | 60088 | 63003 | 50274 |
| pantothenate | 36765 | 47057 | 51757 | 50594 | 83496 | 75706 |
| phosphocholine | 27662 | 17932 | 24148 | 28286 | 18147 | 25205 |
| 2-aminoadipate | 15926 | 21955 | 21783 | 22043 | 21926 | 27445 |
| creatine | 5687 | 13215 | 13837 | 10818 | 21760 | 21343 |
| flavin adenine dinucleotide (FAD) | 50954 | 67986 | 69373 | 62026 | 60735 | 89785 |
| phosphoenolpyruvate (PEP) | 5795 | 6253 | 10323 | 9453 | 15007 | 10168 |
| N-formylmethionine | 4615 | 6413 | 8248 | 5827 | 8439 | 7870 |
| N-acetylmethionine | 5356 | 4733 | 6241 | 6798 | 7338 | 7046 |
| nicotinamide adenine dinucleotide | 788918 | 942330 | 1099003 | 991175 | 1517007 | 1549604 |
| (NAD+) | | | | | | |
| N-acetylglutamate | 13722 | 14070 | 16786 | 18291 | 13363 | 16947 |
| allantoin | 34246 | 40480 | 151837 | 43640 | 53245 | 65534 |
| aspartate | 591073 | 552338 | 642065 | 477378 | 354508 | 306152 |
| taurine | 1337294 | 1140701 | 530522 | 1683227 | 622142 | 1256225 |
| adenosine 5′-monophosphate (AMP) | 105697 | 134289 | 148927 | 166114 | 262825 | 266705 |
| adenosine 5′-diphosphate (ADP) | 41331 | 52634 | 53226 | 85672 | 123523 | 135768 |
| glutathione, reduced (GSH) | 561466 | 1482905 | 886374 | 1129273 | 1664755 | 3016675 |
| glutathione, oxidized (GSSG) | 493214 | 495022 | 603390 | 610178 | 673302 | 951462 |
| sn-glycero-3-phosphoethanolamine | 116664 | 121710 | 100671 | 125396 | 161855 | 177394 |
| UDP-glucuronate | 30602 | 37243 | 40695 | 35881 | 63770 | 54047 |
| N-acetylaspartate (NAA) | 95646 | 82585 | 102479 | 93800 | 78635 | 89508 |
| carbamoyl aspartate | 67898 | 65634 | 65752 | 68117 | 33472 | 43033 |
| glutamate | 1363682 | 1264285 | 376577 | 2239638 | 1367263 | 3253127 |
| UDP-N-acetylglucosamine | 59327 | 64438 | 65159 | 61472 | 88738 | 89106 |
| methylthioribulose 1-phosphate | 57713 | 27470 | 47869 | 51280 | 18403 | 30957 |
| lactate | 587797 | 689660 | 1134925 | 1203384 | 1089096 | 1381886 |
| malate | 83818 | 87527 | 1328292 | 97247 | 90070 | 101499 |
| uridine 5′-monophosphate (UMP) | 33133 | 44732 | 65199 | 60877 | 70581 | 89192 |
| gamma-aminobutyrate (GABA) | 49845 | 49880 | 20918 | 76341 | 53878 | 103864 |
| 5-oxoproline | 187227 | 145544 | 248441 | 256266 | 344922 | 284823 |
| alpha-ketoglutarate | 73521 | 65611 | 85776 | 61895 | 35730 | 68344 |
| phenylacetylglycine | 17512 | 21801 | 26181 | 22733 | 35283 | 41659 |
| S-sulfoglutathione | 233998 | 138986 | 156607 | 179205 | 257454 | 192373 |
| histidine | 172306 | 155971 | 208804 | 282905 | 252162 | 198427 |
| asparagine | 20459 | 14715 | 56130 | 31820 | 27217 | 17490 |
| galactonic acid | 98431 | 108536 | 172704 | 106101 | 134549 | 123062 |
| beta-glycerophosphoric acid | 55253 | 89493 | 101245 | 149250 | 156452 | 217626 |
| methylmalonate | 70008 | 77431 | 87748 | 80647 | 98279 | 93062 |
| succinate | 43470 | 47865 | 54110 | 50861 | 61015 | 59850 |
| serine | 45419 | 23897 | 61854 | 100200 | 80716 | 31146 |
| 2-hydroxyglutarate | 58477 | 42768 | 54287 | 92890 | 65067 | 190529 |
| citrate | 3096953 | 2798713 | 5454761 | 3823024 | 3679408 | 4123862 |
| threonine | 64335 | 31021 | 69157 | 95808 | 121536 | 56979 |
| 3-hydroxyhexadecanoate | 188838 | 157292 | 208426 | 182826 | 204530 | 239719 |
| glutamine | 44712 | 39969 | 68429 | 60915 | 47252 | 58773 |
| fructose 1,6-biphosphate | 52357 | 25907 | 11679 | 164505 | 143458 | 286542 |
| 4-acetylbutyrate | 59403 | 54490 | 69549 | 82253 | 86844 | 91926 |
| adenosine 5′-triphosphate (ATP) | 30663 | 13909 | 6280 | 124998 | 107650 | 276650 |
| threonate | 90348 | 97017 | 80686 | 157247 | 94328 | 144803 |
| uridine 5′-triphosphate (UTP) | 67457 | 35729 | 17452 | 229439 | 226825 | 457108 |
| pentadecanoic acid | 504338 | 549090 | 641712 | 1063710 | 798452 | 1060462 |
| phenylacetate | 354871 | 220785 | 254671 | 281496 | 253015 | 328551 |
| stearate | 28612917 | 27691251 | 33857413 | 38581889 | 41841077 | 44239155 |
| phosphoglycolic acid | 13991 | 11826 | 11630 | 15974 | 18993 | 17888 |
| 3-methyl-2-oxovalerate | 26343 | 25087 | 23458 | 26337 | 28174 | 30302 |
| palmitate | 54167997 | 53180320 | 66741027 | 75595466 | 73809899 | 86011716 |
| oleate | 3692380 | 3657843 | 4488754 | 6766385 | 3584895 | 6287628 |
| dodecanoate | 147082480 | 123190279 | 136493967 | 158661165 | 145967860 | 174736732 |
| caproate | 1754914 | 990905 | 1327517 | 1665598 | 1140351 | 1378443 |
| 3-hydroxy-3-methylglutarate | 81309 | 68400 | 23477 | 74827 | 87940 | 85195 |
| N-acetyl-beta-alanine | 7777 | 100 | 8966 | 11386 | 9375 | 11406 |

TABLE 1F
Metabolites results

| | | | | DLD1- | DLD1- | DLD1- |
|---|---|---|---|---|---|---|
| | DLD1- | DLD1- | DLD1- | OMP25HA | OMP25HA | OMP25HA |
| | OMP25HA | OMP25HA | OMP25HA | sgCtrl + | sgCtrl + | sgCtrl + |
| | sgNADK2-2 | sgNADK2-2 | sgNADK2-2 | Piericidin | Piericidin | Piericidin |
| | MitoIP_{—} | MitoIP_{—} | MitoIP_{—} | MitoIP_{—} | MitoIP_{—} | MitoIP_{—} |
| Metabolite Name | rep01 | rep02 | rep03 | rep01 | rep02 | rep03 |
| 4-hydroxyproline | not | not | not | not | not | not |
| | detected | detected | detected | detected | detected | detected |
| 6-phosphogluconate | not | not | not | not | not | not |
| | detected | detected | detected | detected | detected | detected |
| aconitate | not | not | not | not | not | not |
| | detected | detected | detected | detected | detected | detected |
| alanine | not | not | not | not | not | not |
| | detected | detected | detected | detected | detected | detected |
| arginine | not | not | not | not | not | not |
| | detected | detected | detected | detected | detected | detected |
| betaine | not | not | not | not | not | not |
| | detected | detected | detected | detected | detected | detected |
| carnitine | not | not | not | not | not | not |
| | detected | detected | detected | detected | detected | detected |
| creatine phosphate | not | not | not | not | not | not |
| | detected | detected | detected | detected | detected | detected |
| creatinine | not | not | not | not | not | not |
| | detected | detected | detected | detected | detected | detected |
| cytidine 5′-triphosphate (CTP) | not | not | not | not | not | not |
| | detected | detected | detected | detected | detected | detected |
| dihydroxyacetone phosphate (DHAP) | not | not | not | not | not | not |
| | detected | detected | detected | detected | detected | detected |
| formylpyruvate | not | not | not | not | not | not |
| | detected | detected | detected | detected | detected | detected |
| fructose 6′-phosphate | not | not | not | not | not | not |
| | detected | detected | detected | detected | detected | detected |
| fumarate | not | not | not | not | not | not |
| | detected | detected | detected | detected | detected | detected |
| glucosamine | not | not | not | not | not | not |
| | detected | detected | detected | detected | detected | detected |
| glycerophosphorylcholine (GPC) | not | not | not | not | not | not |
| | detected | detected | detected | detected | detected | detected |
| glycine | not | not | not | not | not | not |
| | detected | detected | detected | detected | detected | detected |
| guanosine 5′-diphosphate (GDP) | not | not | not | not | not | not |
| | detected | detected | detected | detected | detected | detected |
| guanosine 5′-diphosphoglucose | not | not | not | not | not | not |
| | detected | detected | detected | detected | detected | detected |
| guanosine 5′-monophosphate (GMP) | not | not | not | not | not | not |
| | detected | detected | detected | detected | detected | detected |
| guanosine 5′-triphosphate (GTP) | not | not | not | not | not | not |
| | detected | detected | detected | detected | detected | detected |
| hydroxyphenyllactate | not | not | not | not | not | not |
| | detected | detected | detected | detected | detected | detected |
| inosine 5′-monophosphate (IMP) | not | not | not | not | not | not |
| | detected | detected | detected | detected | detected | detected |
| isoleucine | not | not | not | not | not | not |
| | detected | detected | detected | detected | detected | detected |
| leucine | not | not | not | not | not | not |
| | detected | detected | detected | detected | detected | detected |
| lysine | not | not | not | not | not | not |
| | detected | detected | detected | detected | detected | detected |
| methionine | not | not | not | not | not | not |
| | detected | detected | detected | detected | detected | detected |
| N6-(delta-isopentenyl)-adenine | not | not | not | not | not | not |
| | detected | detected | detected | detected | detected | detected |
| N-acetylalanine | not | not | not | not | not | not |
| | detected | detected | detected | detected | detected | detected |
| N-acetylglycine | not | not | not | not | not | not |
| | detected | detected | detected | detected | detected | detected |
| N-acetylserine | not | not | not | not | not | not |
| | detected | detected | detected | detected | detected | detected |
| nicotinamide | not | not | not | not | not | not |
| | detected | detected | detected | detected | detected | detected |
| nicotinamide adenine ainucleotide | not | not | not | not | not | not |
| phosphate, reduced (NADPH) | detected | detected | detected | detected | detected | detected |
| nicotinamide adenine dinucleotide | not | not | not | not | not | not |
| phosphate (NADP+) | detected | detected | detected | detected | detected | detected |
| proline | not | not | not | not | not | not |
| | detected | detected | detected | detected | detected | detected |
| ribose 5′-phosphate | not | not | not | not | not | not |
| | detected | detected | detected | detected | detected | detected |
| thymidine 5′-diphospho-alpha-D- | not | not | not | not | not | not |
| glucose | detected | detected | detected | detected | detected | detected |
| thymine | not | not | not | not | not | not |
| | detected | detected | detected | detected | detected | detected |
| tryptophan | not | not | not | not | not | not |
| | detected | detected | detected | detected | detected | detected |
| tyrosine | not | not | not | not | not | not |
| | detected | detected | detected | detected | detected | detected |
| uridine 5′-diphosphate (UDP) | not | not | not | not | not | not |
| | detected | detected | detected | detected | detected | detected |
| valine | not | not | not | not | not | not |
| | detected | detected | detected | detected | detected | detected |
| N-acetylglucosamine 6-phosphate | not | not | not | not | not | not |
| | detected | detected | detected | detected | detected | detected |
| N-acetylthreonine | not | not | not | not | not | not |
| | detected | detected | detected | detected | detected | detected |
| galactitol | not | not | not | not | not | not |
| | detected | detected | detected | detected | detected | detected |
| alanylhistidine | not | not | not | not | not | not |
| | detected | detected | detected | detected | detected | detected |
| S-adenosylhomocysteine (SAH) | not | not | not | not | not | not |
| | detected | detected | detected | detected | detected | detected |
| serinylaspartate | not | not | not | not | not | not |
| | detected | detected | detected | detected | detected | detected |
| adenine | not | not | not | not | not | not |
| | detected | detected | detected | detected | detected | detected |
| folate | not | not | not | not | not | not |
| | detected | detected | detected | detected | detected | detected |
| glycylaspartate | not | not | not | not | not | not |
| | detected | detected | detected | detected | detected | detected |
| glyceraldehyde 3-phosphate | not | not | not | not | not | not |
| | detected | detected | detected | detected | detected | detected |
| 5-methylthioadenosine (MTA) | not | not | not | not | not | not |
| | detected | detected | detected | detected | detected | detected |
| N-acetyltaurine | not | not | not | not | not | not |
| | detected | detected | detected | detected | detected | detected |
| glucose 6′-phosphate | not | not | not | not | not | not |
| | detected | detected | detected | detected | detected | detected |
| nicotinamide adenine dinucleotide, | 539863 | 461557 | 244855 | 3526961 | 3705811 | 4542289 |
| reduced (NADH) | | | | | | |
| phenylalanine | 68445 | 39707 | 40131 | 128095 | 81873 | 174756 |
| pantothenate | 52729 | 82642 | 55627 | 132084 | 136875 | 67947 |
| phosphocholine | 35680 | 24446 | 13977 | 31184 | 30344 | 102365 |
| 2-aminoadipate | 25588 | 24794 | 15328 | 62966 | 65899 | 79200 |
| creatine | 13643 | 24535 | 11844 | 33063 | 35737 | 83791 |
| flavin adenine dinucleotide (FAD) | 70115 | 71140 | 49018 | 92983 | 61170 | 160187 |
| phosphoenolpyruvate (PEP) | 13741 | 9192 | 3515 | 1592 | 100 | 7511 |
| N-formylmethionine | 7862 | 9019 | 3529 | 13879 | 14361 | 41248 |
| N-acetylmethionine | 8815 | 9234 | 4999 | 13634 | 16094 | 39435 |
| nicotinamide adenine dinucleotide | 1185175 | 1770562 | 707924 | 854478 | 683438 | 3220536 |
| (NAD+) | | | | | | |
| N-acetylglutamate | 19421 | 24104 | 11138 | 22549 | 22110 | 46571 |
| allantoin | 47712 | 84508 | 38279 | 95330 | 84331 | 462241 |
| aspartate | 469496 | 401136 | 180941 | 220307 | 78505 | 48863 |
| taurine | 1502607 | 1315319 | 1193153 | 3028424 | 3328577 | 3010797 |
| adenosine 5′-monophosphate (AMP) | 216539 | 264908 | 134589 | 337267 | 297007 | 605531 |
| adenosine 5′-diphosphate (ADP) | 154514 | 63116 | 26057 | 87866 | 78786 | 255009 |
| glutathione, reduced (GSH) | 3239739 | 1657083 | 83552 | 5666886 | 8127441 | 4704089 |
| glutathione, oxidized (GSSG) | 738239 | 837198 | 471567 | 871859 | 648911 | 2509570 |
| sn-glycero-3-phosphoethanolamine | 139445 | 201594 | 115884 | 183356 | 166729 | 403493 |
| UDP-glucuronate | 51735 | 46895 | 27397 | 52926 | 58672 | 185814 |
| N-acetylaspartate (NAA) | 92137 | 95275 | 57514 | 143230 | 135619 | 280527 |
| carbamoyl aspartate | 94132 | 42849 | 24163 | 61946 | 44834 | 50496 |
| glutamate | 3187966 | 2100839 | 1913157 | 9279200 | 8652279 | 19502944 |
| UDP-N-acetylglucosamine | 73562 | 86505 | 68251 | 74573 | 76201 | 164456 |
| methylthioribulose 1-phosphate | 60674 | 21883 | 20110 | 78919 | 79663 | 140471 |
| lactate | 999203 | 1416051 | 1150664 | 2355878 | 2582709 | 1383509 |
| malate | 116880 | 76317 | 70416 | 78861 | 87997 | 101969 |
| uridine 5′-monophosphate (UMP) | 75525 | 75824 | 42988 | 149496 | 116561 | 209899 |
| gamma-aminobutyrate (GABA) | 98333 | 71540 | 57969 | 250114 | 230022 | 464298 |
| 5-oxoproline | 247133 | 283858 | 359581 | 708770 | 454060 | 353025 |
| alpha-ketoglutarate | 54029 | 64107 | 60815 | 36956 | 33481 | 64213 |
| phenylacetylglycine | 22559 | 47977 | 25147 | 42413 | 44122 | 160447 |
| S-sulfoglutathione | 131542 | 369939 | 407077 | 94779 | 78896 | 455627 |
| histidine | 184068 | 222324 | 186176 | 1153876 | 442480 | 686681 |
| asparagine | 18533 | 21272 | 32398 | 117961 | 37108 | 29078 |
| galactonic acid | 122095 | 136255 | 77865 | 79663 | 109571 | 76436 |
| beta-glycerophosphoric acid | 124791 | 243518 | 115328 | 242620 | 122348 | 400912 |
| methylmalonate | 88336 | 119579 | 63546 | 49637 | 60988 | 87945 |
| succinate | 55435 | 68943 | 37745 | 32755 | 39006 | 43999 |
| serine | 38421 | 61139 | 107952 | 453038 | 109734 | 30402 |
| 2-hydroxyglutarate | 92298 | 121784 | 187455 | 201668 | 129941 | 381356 |
| citrate | 4357860 | 2489481 | 2291452 | 2652773 | 2956510 | 3197992 |
| threonine | 75918 | 66108 | 88425 | 200140 | 69914 | 45369 |
| 3-hydroxyhexadecanoate | 193903 | 192249 | 156816 | 154313 | 165893 | 273073 |
| glutamine | 57493 | 49329 | 41813 | 58654 | 57894 | 67465 |
| fructose 1,6-biphosphate | 346613 | 21493 | 12912 | 19416 | 35392 | 701747 |
| 4-acetylbutyrate | 74543 | 95487 | 59501 | 47845 | 59291 | 180176 |
| adenosine 5′-triphosphate (ATP) | 269251 | 11656 | 6897 | 11535 | 22764 | 745088 |
| threonate | 93142 | 91048 | 76593 | 83355 | 117396 | 46647 |
| uridine 5′-triphosphate (UTP) | 419633 | 30590 | 3348 | 28230 | 52737 | 1159604 |
| pentadecanoic acid | 741196 | 789709 | 683993 | 988055 | 823203 | 616849 |
| phenylacetate | 243426 | 359356 | 296795 | 206078 | 155618 | 258772 |
| stearate | 32367654 | 30545573 | 28439719 | 32097131 | 29146976 | 11388935 |
| phosphoglycolic acid | 21222 | 12919 | 8937 | 12625 | 17645 | 24712 |
| 3-methyl-2-oxovalerate | 28711 | 32223 | 25061 | 22054 | 26080 | 71467 |
| palmitate | 62874514 | 70531040 | 55205500 | 58619197 | 59278003 | 34887756 |
| oleate | 4476126 | 5143469 | 3285091 | 3976796 | 4283346 | 4070066 |
| dodecanoate | 134931202 | 164597660 | 132377821 | 117632035 | 108567664 | 182339246 |
| caproate | 1077070 | 1744540 | 1490124 | 856796 | 553769 | 1465682 |
| 3-hydroxy-3-methylglutarate | 65754 | 76609 | 62575 | 65452 | 77263 | 90857 |
| N-acetyl-beta-alanine | 10010 | 7584 | 7740 | 11958 | 12004 | 29818 |

TABLE 1G
Metabolites results

| | | | | | FC |
|---|---|---|---|---|---|
| | | | Detected | | ([OMP25HA |
| | | | in | Detected | sgCtrl |
| | DLD1-OMP25 | DLD1-OMP25 | whole | in | MitoIP] Vs |
| | Myc | Myc | cell | MitoI P | [OMP25Myc |
| Metabolite Name | MitoIP_rep01 | MitoIP_rep02 | samples | samples | MitoIP]) |
| 4-hydroxyproline | not | not | TRUE | FALSE | not |
| | detected | detected | | | applicable |
| 6-phosphogluconate | not | not | TRUE | FALSE | not |
| | detected | detected | | | applicable |
| aconitate | not | not | TRUE | FALSE | not |
| | detected | detected | | | applicable |
| alanine | not | not | TRUE | FALSE | not |
| | detected | detected | | | applicable |
| arginine | not | not | TRUE | FALSE | not |
| | detected | detected | | | applicable |
| betaine | not | not | TRUE | FALSE | not |
| | detected | detected | | | applicable |
| carnitine | not | not | TRUE | FALSE | not |
| | detected | detected | | | applicable |
| creatine phosphate | not | not | TRUE | FALSE | not |
| | detected | detected | | | applicable |
| creatinine | not | not | TRUE | FALSE | not |
| | detected | detected | | | applicable |
| cytidine 5′-triphosphate | not | not | TRUE | FALSE | not |
| (CTP) | detected | detected | | | applicable |
| dihydroxyacetone phosphate | not | not | TRUE | FALSE | not |
| (DHAP) | detected | detected | | | applicable |
| formylpyruvate | not | not | TRUE | FALSE | not |
| | detected | detected | | | applicable |
| fructose 6′-phosphate | not | not | TRUE | FALSE | not |
| | detected | detected | | | applicable |
| fumarate | not | not | TRUE | FALSE | not |
| | detected | detected | | | applicable |
| glucosamine | not | not | TRUE | FALSE | not |
| | detected | detected | | | applicable |
| glycerophosphorylcholine (GPC) | not | not | TRUE | FALSE | not |
| | detected | detected | | | applicable |
| glycine | not | not | TRUE | FALSE | not |
| | detected | detected | | | applicable |
| guanosine 5′-diphosphate | not | not | TRUE | FALSE | not |
| (GDP) | detected | detected | | | applicable |
| guanosine 5′-diphosphoglucose | not | not | TRUE | FALSE | not |
| | detected | detected | | | applicable |
| guanosine 5′-monophosphate | not | not | TRUE | FALSE | not |
| (GMP) | detected | detected | | | applicable |
| guanosine 5′-triphosphate | not | not | TRUE | FALSE | not |
| (GTP) | detected | detected | | | applicable |
| hydroxyphenyllactate | not | not | TRUE | FALSE | not |
| | detected | detected | | | applicable |
| inosine 5′-monophosphate | not | not | TRUE | FALSE | not |
| (IMP) | detected | detected | | | applicable |
| isoleucine | not | not | TRUE | FALSE | not |
| | detected | detected | | | applicable |
| leucine | not | not | TRUE | FALSE | not |
| | detected | detected | | | applicable |
| lysine | not | not | TRUE | FALSE | not |
| | detected | detected | | | applicable |
| methionine | not | not | TRUE | FALSE | not |
| | detected | detected | | | applicable |
| N6-(delta-isopentenyl)-adenine | not | not | TRUE | FALSE | not |
| | detected | detected | | | applicable |
| N-acetylalanine | not | not | TRUE | FALSE | not |
| | detected | detected | | | applicable |
| N-acetylglycine | not | not | TRUE | FALSE | not |
| | detected | detected | | | applicable |
| N-acetylserine | not | not | TRUE | FALSE | not |
| | detected | detected | | | applicable |
| nicotinamide | not | not | TRUE | FALSE | not |
| | detected | detected | | | applicable |
| nicotinamide adenine ainucleotide | not | not | TRUE | FALSE | not |
| phosphate, reduced (NADPH) | detected | detected | | | applicable |
| nicotinamide adenine dinucleotide | not | not | TRUE | FALSE | not |
| phosphate (NADP+) | detected | detected | | | applicable |
| proline | not | not | TRUE | FALSE | not |
| | detected | detected | | | applicable |
| ribose 5′-phosphate | not | not | TRUE | FALSE | not |
| | detected | detected | | | applicable |
| thymidine 5′-diphospho-alpha- | not | not | TRUE | FALSE | not |
| D-glucose | detected | detected | | | applicable |
| thymine | not | not | TRUE | FALSE | not |
| | detected | detected | | | applicable |
| tryptophan | not | not | TRUE | FALSE | not |
| | detected | detected | | | applicable |
| tyrosine | not | not | TRUE | FALSE | not |
| | detected | detected | | | applicable |
| uridine 5′-diphosphate (UDP) | not | not | TRUE | FALSE | not |
| | detected | detected | | | applicable |
| valine | not | not | TRUE | FALSE | not |
| | detected | detected | | | applicable |
| N-acetylglucosamine 6-phosphate | not | not | TRUE | FALSE | not |
| | detected | detected | | | applicable |
| N-acetylthreonine | not | not | TRUE | FALSE | not |
| | detected | detected | | | applicable |
| galactitol | not | not | TRUE | FALSE | not |
| | detected | detected | | | applicable |
| alanylhistidine | not | not | TRUE | FALSE | not |
| | detected | detected | | | applicable |
| S-adenosylhomocysteine (SAH) | not | not | TRUE | FALSE | not |
| | detected | detected | | | applicable |
| serinylaspartate | not | not | TRUE | FALSE | not |
| | detected | detected | | | applicable |
| adenine | not | not | TRUE | FALSE | not |
| | detected | detected | | | applicable |
| folate | not | not | TRUE | FALSE | not |
| | detected | detected | | | applicable |
| glycylaspartate | not | not | TRUE | FALSE | not |
| | detected | detected | | | applicable |
| glyceraldehyde 3-phosphate | not | not | TRUE | FALSE | not |
| | detected | detected | | | applicable |
| 5-methylthioadenosine (MTA) | not | not | TRUE | FALSE | not |
| | detected | detected | | | applicable |
| N-acetyltaurine | not | not | TRUE | FALSE | not |
| | detected | detected | | | applicable |
| glucose 6′-phosphate | not | not | TRUE | FALSE | not |
| | detected | detected | | | applicable |
| nicotinamide adenine dinucleotide, | 100 | 100 | TRUE | TRUE | 4714.50 |
| reduced (NADH) | | | | | |
| phenylalanine | 100 | 100 | TRUE | TRUE | 448.94 |
| pantothenate | 100 | 100 | TRUE | TRUE | 447.38 |
| phosphocholine | 100 | 100 | TRUE | TRUE | 228.81 |
| 2-aminoadipate | 297 | 100 | TRUE | TRUE | 114.15 |
| creatine | 100 | 100 | TRUE | TRUE | 101.31 |
| flavin adenine dinucleotide (FAD) | 6484 | 100 | TRUE | TRUE | 77.21 |
| phosphoenolpyruvate (PEP) | 100 | 100 | TRUE | TRUE | 72.05 |
| N-formylmethionine | 100 | 100 | TRUE | TRUE | 62.50 |
| N-acetylmethionine | 100 | 100 | TRUE | TRUE | 54.09 |
| nicotinamide adenine dinucleotide | 15570 | 30587 | TRUE | TRUE | 42.84 |
| (NAD+) | | | | | |
| N-acetylglutamate | 682 | 571 | TRUE | TRUE | 23.72 |
| allantoin | 6320 | 2667 | TRUE | TRUE | 14.49 |
| aspartate | 53023 | 36663 | TRUE | TRUE | 13.47 |
| taurine | 66639 | 77634 | TRUE | TRUE | 12.96 |
| adenosine 5′-monophosphate (AMP) | 10072 | 10760 | TRUE | TRUE | 12.33 |
| adenosine 5′-diphosphate (ADP) | 6475 | 2971 | TRUE | TRUE | 11.11 |
| glutathione, reduced (GSH) | 110603 | 83110 | TRUE | TRUE | 9.43 |
| glutathione, oxidized (GSSG) | 70250 | 56519 | TRUE | TRUE | 8.38 |
| sn-glycero-3-phosphoethanolamine | 14122 | 13811 | TRUE | TRUE | 8.07 |
| UDP-glucuronate | 2619 | 7637 | TRUE | TRUE | 8.03 |
| N-acetylaspartate (NAA) | 15269 | 15471 | TRUE | TRUE | 6.06 |
| carbamoyl aspartate | 11119 | 10907 | TRUE | TRUE | 6.03 |
| glutamate | 185310 | 134667 | TRUE | TRUE | 5.48 |
| UDP-N-acetylglucosamine | 9662 | 14245 | TRUE | TRUE | 5.36 |
| methylthioribulose 1-phosphate | 8147 | 8284 | TRUE | TRUE | 5.15 |
| lactate | 196345 | 129197 | TRUE | TRUE | 4.85 |
| malate | 46804 | 49448 | TRUE | TRUE | 4.44 |
| uridine 5′-monophosphate (UMP) | 11085 | 12670 | TRUE | TRUE | 3.87 |
| gamma-aminobutyrate (GABA) | 10932 | 9596 | TRUE | TRUE | 3.64 |
| 5-oxoproline | 60368 | 51498 | TRUE | TRUE | 3.39 |
| alpha-ketoglutarate | 24972 | 21210 | TRUE | TRUE | 3.24 |
| phenylacetylglycine | 7945 | 8250 | TRUE | TRUE | 2.66 |
| S-sulfoglutathione | 77390 | 58986 | TRUE | TRUE | 2.55 |
| histidine | 77945 | 82637 | TRUE | TRUE | 2.21 |
| asparagine | 13472 | 10891 | TRUE | TRUE | 2.12 |
| galactonic acid | 69295 | 51697 | TRUE | TRUE | 2.05 |
| beta-glycerophosphoric acid | 35362 | 51030 | TRUE | TRUE | 1.87 |
| methylmalonate | 49396 | 41669 | TRUE | TRUE | 1.72 |
| succinate | 31781 | 24803 | TRUE | TRUE | 1.72 |
| serine | 26226 | 22671 | TRUE | TRUE | 1.67 |
| 2-hydroxyglutarate | 38897 | 28066 | TRUE | TRUE | 1.56 |
| citrate | 2719007 | 2374693 | TRUE | TRUE | 1.42 |
| threonine | 40101 | 37277 | TRUE | TRUE | 1.34 |
| 3-hydroxyhexadecanoate | 158760 | 134376 | TRUE | TRUE | 1.26 |
| glutamine | 46036 | 34254 | TRUE | TRUE | 1.25 |
| fructose 1,6-biphosphate | 29250 | 15119 | TRUE | TRUE | 1.19 |
| 4-acetylbutyrate | 56749 | 46184 | TRUE | TRUE | 1.19 |
| adenosine 5′-triphosphate (ATP) | 17673 | 7801 | TRUE | TRUE | 1.18 |
| threonate | 94444 | 64011 | TRUE | TRUE | 1.15 |
| uridine 5′-triphosphate (UTP) | 43695 | 21855 | TRUE | TRUE | 1.13 |
| pentadecanoic acid | 533659 | 490120 | TRUE | TRUE | 1.10 |
| phenylacetate | 356354 | 184274 | TRUE | TRUE | 1.06 |
| stearate | 29066677 | 29374263 | TRUE | TRUE | 1.02 |
| phosphoglycolic acid | 14280 | 10999 | TRUE | TRUE | −1.01 |
| 3-methyl-2-oxovalerate | 25970 | 24864 | TRUE | TRUE | −1.02 |
| palmitate | 62835669 | 55134542 | TRUE | TRUE | −1.02 |
| oleate | 4030255 | 4016551 | TRUE | TRUE | −1.02 |
| dodecanoate | 165115954 | 138791997 | TRUE | TRUE | −1.12 |
| caproate | 1846347 | 1210209 | TRUE | TRUE | −1.13 |
| 3-hydroxy-3-methylglutarate | 82711 | 67718 | TRUE | TRUE | −1.48 |
| N-acetyl-beta-alanine | 2760 | 2451 | TRUE | TRUE | −6.32 |

Oncogenic mutant forms of isocitrate dehydrogenase 1 (IDH1) and isocitrate dehydrogenase 2 (IDH2) require cytosolic and mitochondrial NADPH, respectively, to produce 2-hydroxyglutarate (2HG) from α-ketoglutarate (αKG) (8) (FIG. 5O). The NADK2 gene was deleted in chondrosarcoma cell lines that had either an endogenous IDH1 R132 mutation (JJ012 cells) or IDH2 R172 mutation (CS1 cells) (FIG. 1D). Loss of NADK2 resulted in reduced 2HG abundance (P<0.001) in CS1 cells, but not in JJ012 cells (FIGS. 1E-1F). Control and NADK2-deleted CS1 cells were then subjected to a xenograft tumor assay in vivo and similarly decreased 2HG abundance was observed in tumors formed by NADK2 knockout cells (FIG. 1G). These results confirmed that NADK2 is required to maintain the mitochondrial NADP(H) pool.

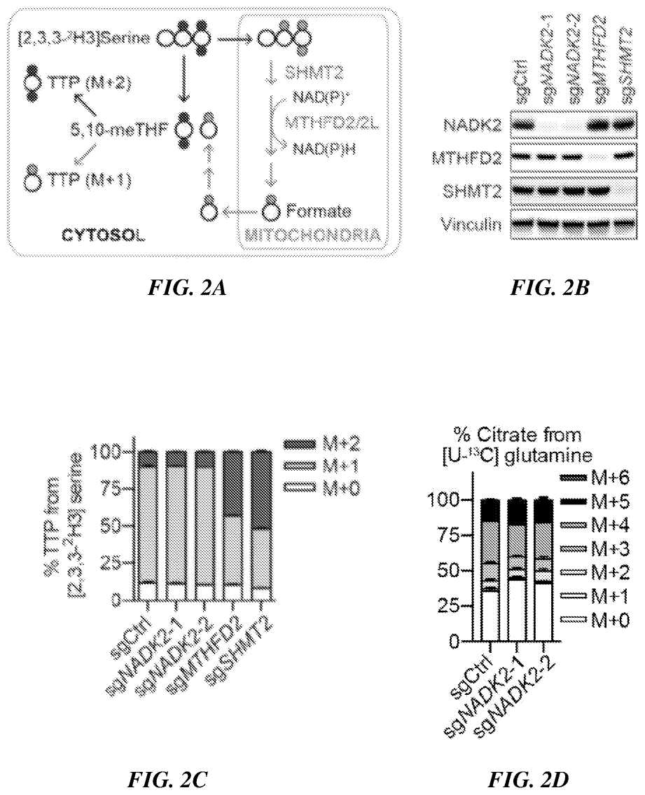

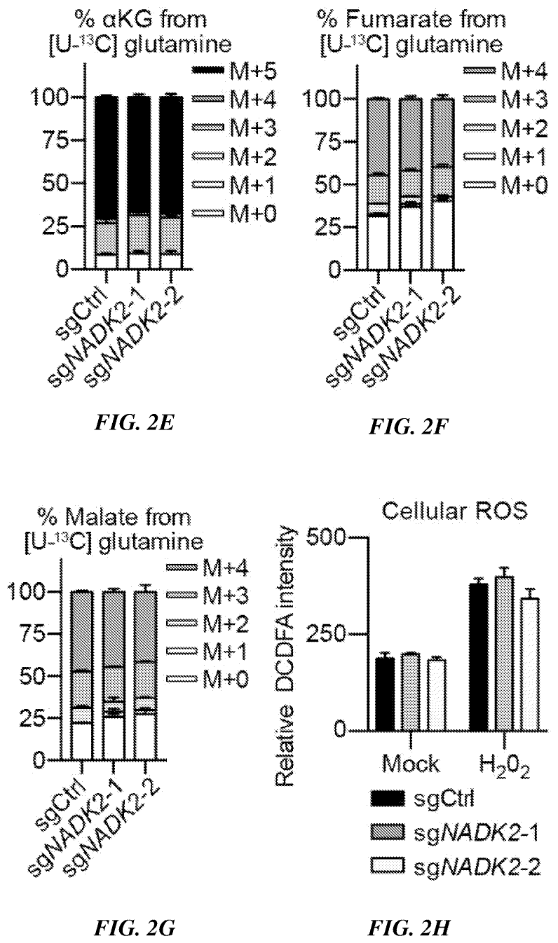

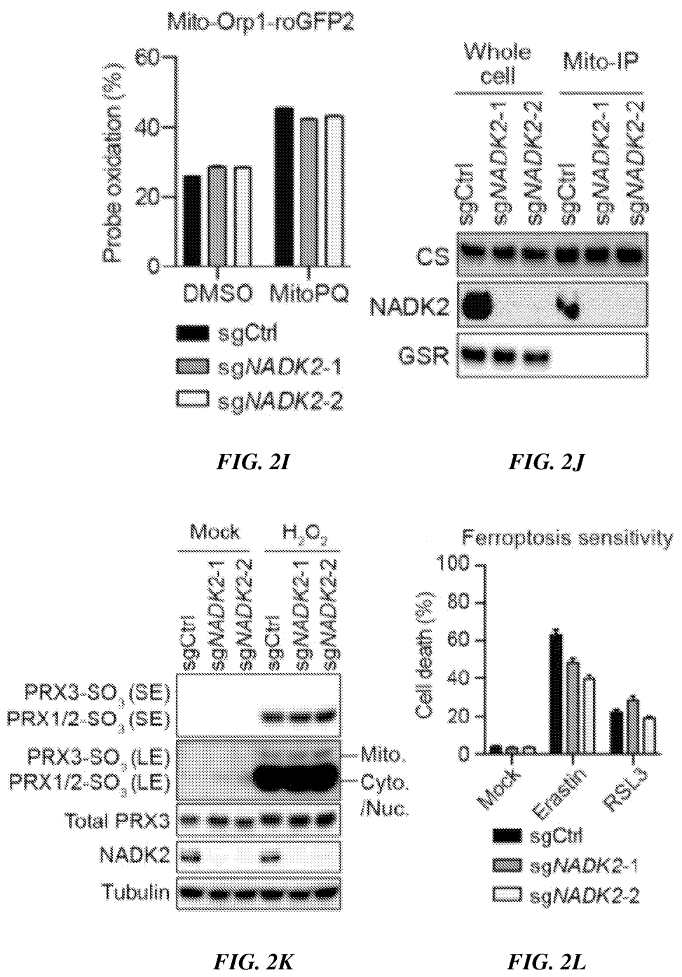

Methylenetetrahydrofolate dehydrogenase 2 (MTHFD2) and MTHFD2-like (MTHFD2L) use either NAD⁺ or NADP⁺as electron acceptors in the mitochondrial folate pathway. Using [2,3,3-2H3]serine isotope tracing, cells lacking MTHFD2 or serine hydroxymethyltransferase 2 (SHMT2) both displayed an increase in doubly-labeled thymidine triphosphate (TTP M+2) when compared to control cells (FIGS. 2A-2C; 6-6B), suggesting decreased mitochondrial folate pathway activity and increased cytosolic serine catabolismas previously reported (9, 10). By contrast, cells lacking NADK2 maintained the fraction of singly labeled (TTP M+1) derived from [2,3,3-2H3]serine (FIGS. 2A-2C; 6-6B), indicating the mitochondrial folate pathway is not disrupted by NADK2 loss.

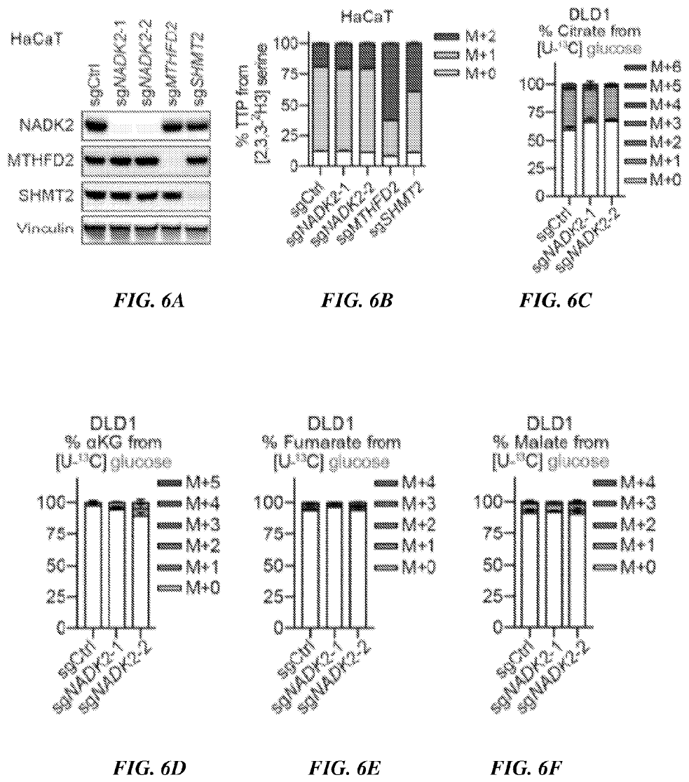

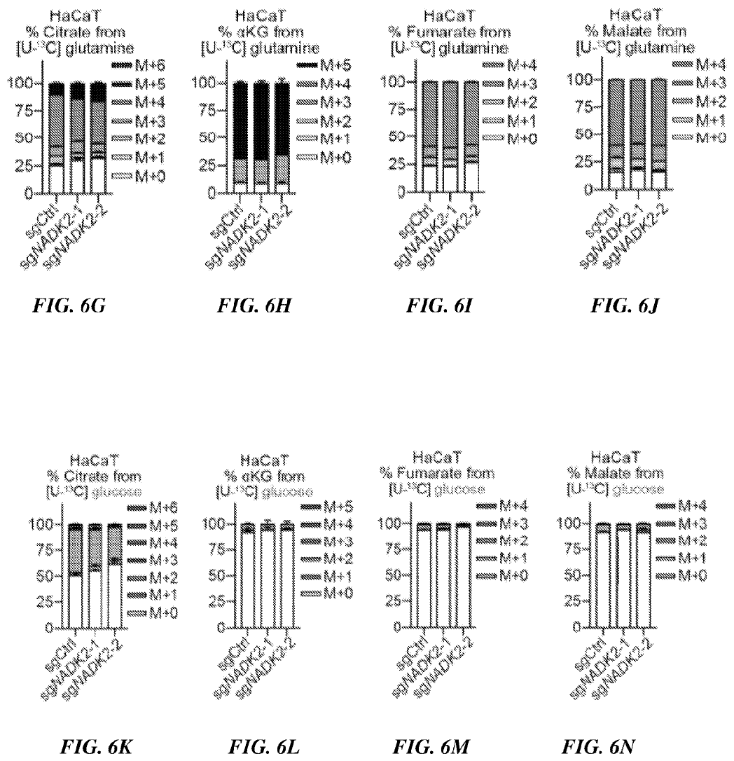

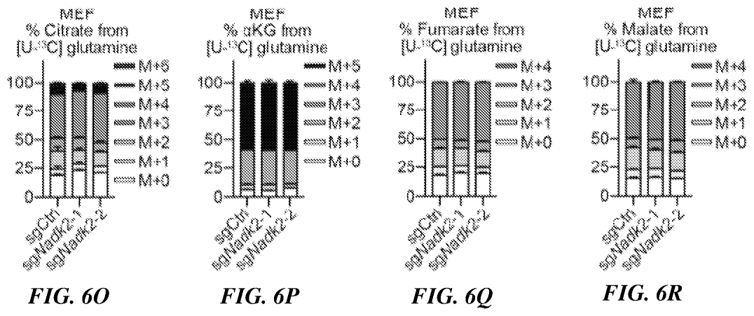

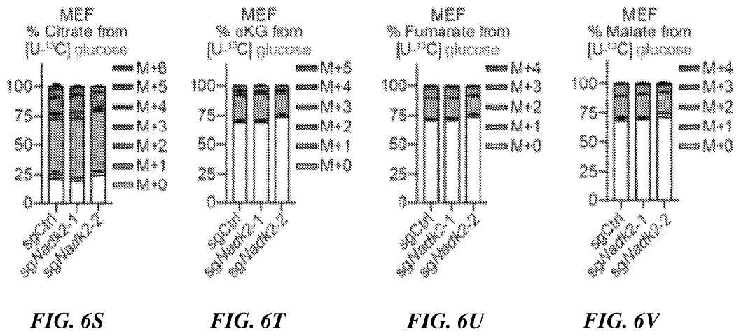

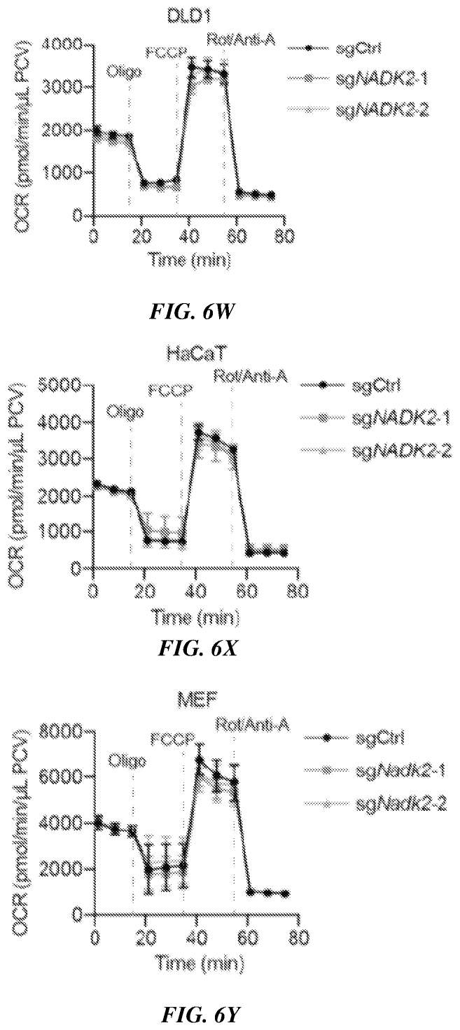

Isotope tracing experiments were performed with uniformly labeled [U-¹³C]glucose or [U-¹³C]glutamine comparing control and NADK2-deleted cells to analyze tricarboxylic acid (TCA) cycle activity. Consistent changes were not observed in the TCA cycle intermediates derived from either glucose or glutamine (FIGS. 2D-2G; 6C-6V). In addition, NADK2-deletion did not lead to changes in the mitochondrial basal oxygen consumption rate or uncoupled electron transport chain activity (FIG. 6W-6Y).

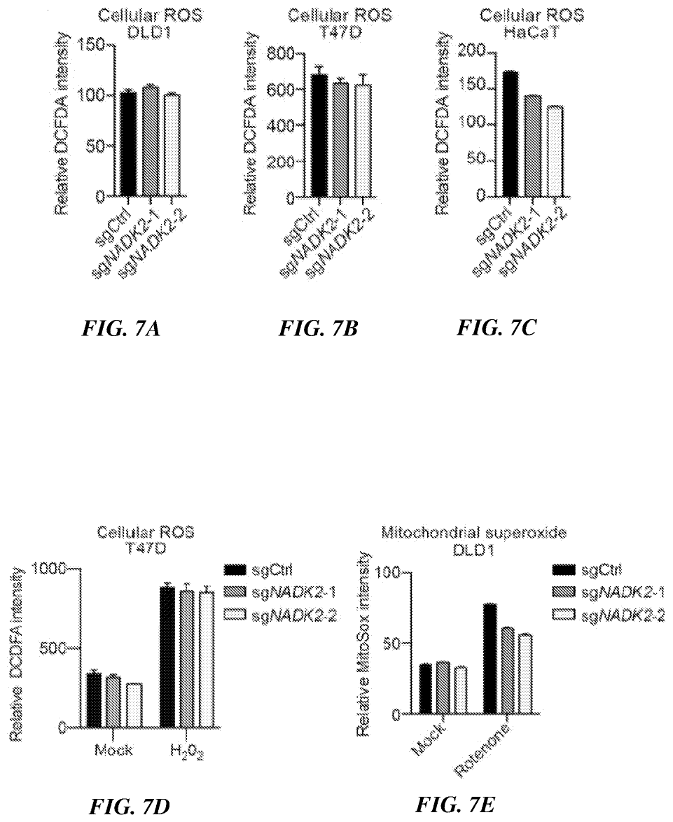

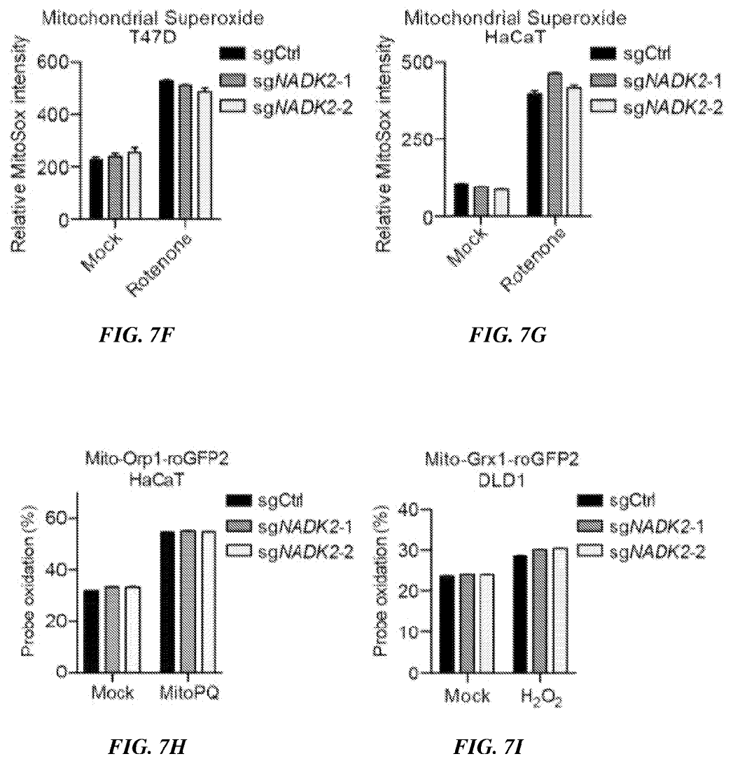

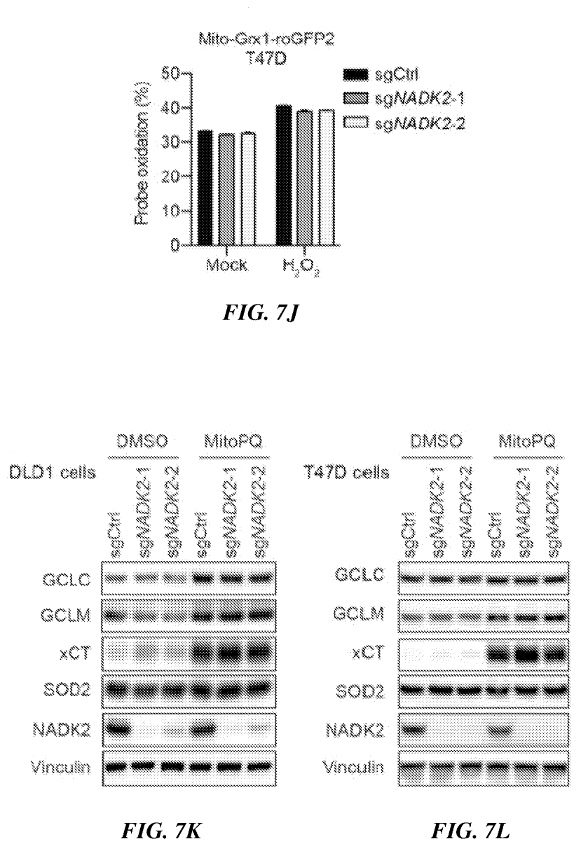

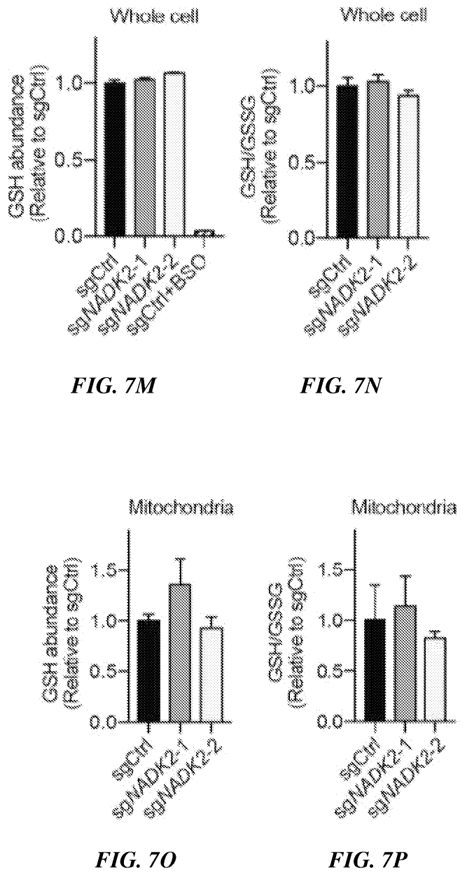

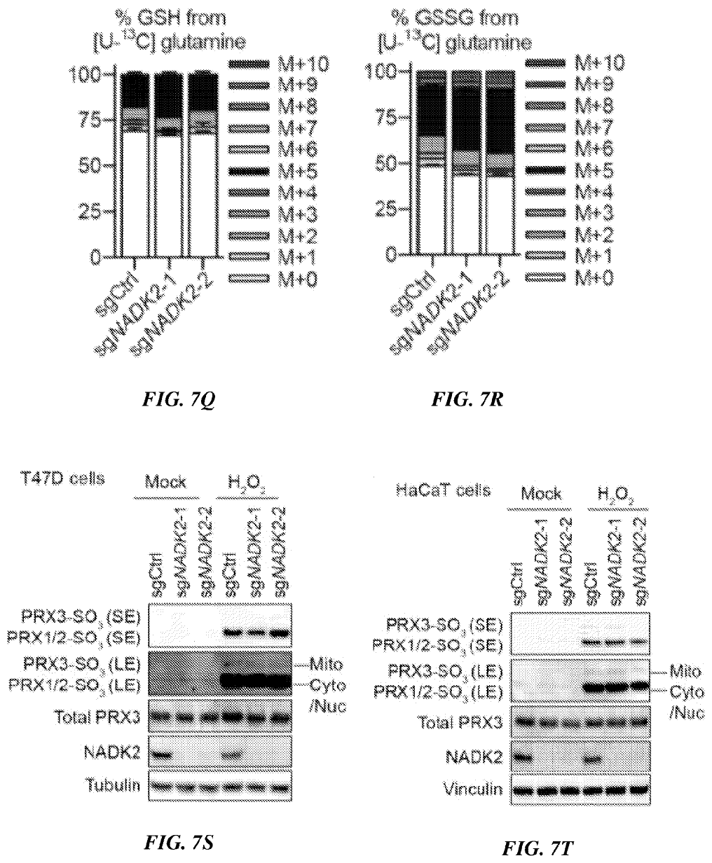

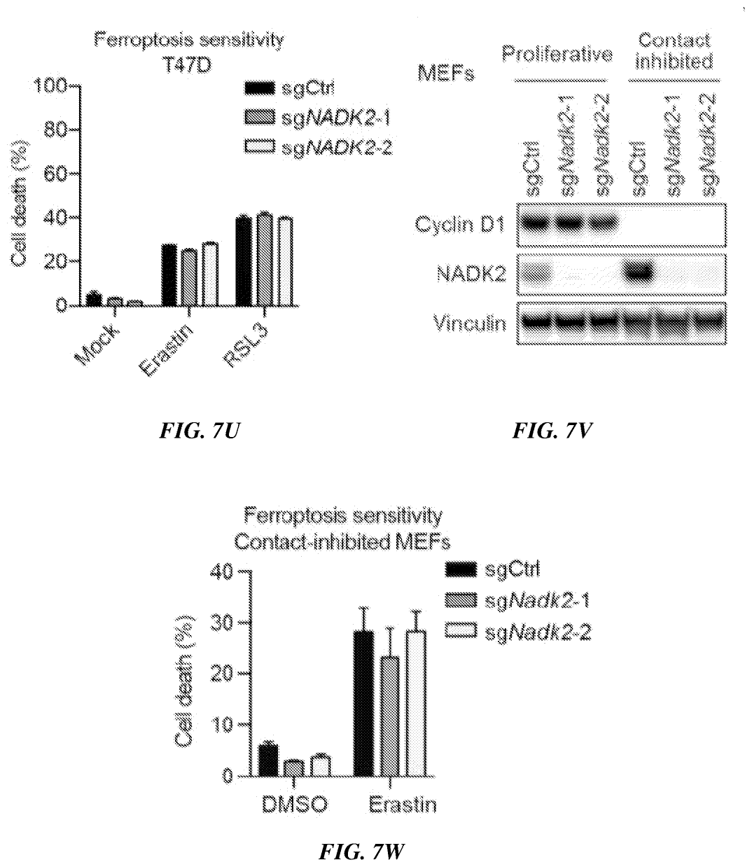

Mitochondria are major sites of reactive oxygen species (ROS) generation in cells (11), and depletion of mitochondrial NADP(H) is thought to lead to oxidative stress. However, in all cell types that were tested, cells lacking NADK2 did not display increased cellular ROS or mitochondrial superoxide (MitoSox) abundance (FIGS. 2H, 7A-7G). Mitochondria-targeted redox-sensitive green fluorescence protein (roGFP2) constructs were used that are coupled to the yeast peroxidase Orp1 or human glutaredoxin-1 (Grx1) (12, 13), and similar amounts of mitochondrial hydrogen peroxide (H₂O₂) or glutathione (GSH) oxidation, respectively, were measured in control and NADK2 knockout cells (FIG. 2I; 7H-7J). Treatment with MitoParaquat (MitoPQ) increased the expression of enzymes involved in GSH synthesis to a similar extent in cells lacking NADK2 as that in the control cells (14) (FIGS. 7K-7L). In agreement, loss of NADK2 did not alter cellular or mitochondrial GSH abundance or the ratio of GSH to its oxidized form glutathione disulfide (GSSG) (GSH/GSSG) (FIGS. 7M-7P). [U-¹³C]glutamine tracing revealed no significant changes in the fraction of GSH or GSSG derived from glutamine upon NADK2 loss (FIGS. 7Q-7R). These results are consistent with the cytosolic NADP(H) pool, but not mitochondrial NADP(H), being critical for maintaining cellular GSH levels to prevent oxidative damage (7). Glutathione reductase (GSR) expression was absent in the mitochondrial fraction (FIG. 2J), thus the NADPH-dependent GSH reduction appears not to take place in mitochondria.

Hyper-oxidation of peroxiredoxins (PRXs-SO₃) indicates oxidative stress of the cellular thioredoxin system. Similar amounts of mitochondrial peroxiredoxin (PRX3) were observed, as well as cytosolic (PRX1) and nuclear (PRX2) peroxiredoxin oxidation, when comparing cells lacking NADK2 with control cells (FIGS. 2K, 7S-7T). Cellular and mitochondrial oxidative stress can lead to ferroptotic cell death (15, 16). When treated with Erastin or RSL3, chemicals that induce ferroptosis, cells lacking NADK2 showed no increase in cell death (FIG. 2L, FIG. 7U). Similarly, NADK2 knockout did not increase sensitivity to ferroptosis in contact-inhibited, non-proliferative mouse embryonic fibroblasts (MEFs) (FIGS. 7V-7W). Thus, loss of NADK2, and depletion of mitochondrial NADP(H), did not increase oxidative stress under the experimental conditions examined, although it remains possible that mitochondrial NADP(H) generation might play a role in antioxidant defense in response to other physiological perturbations.

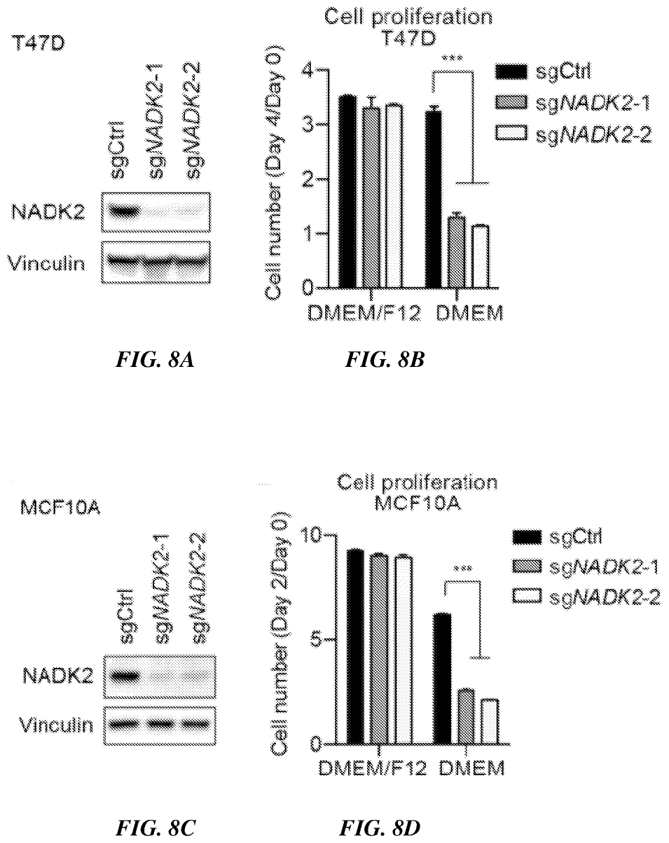

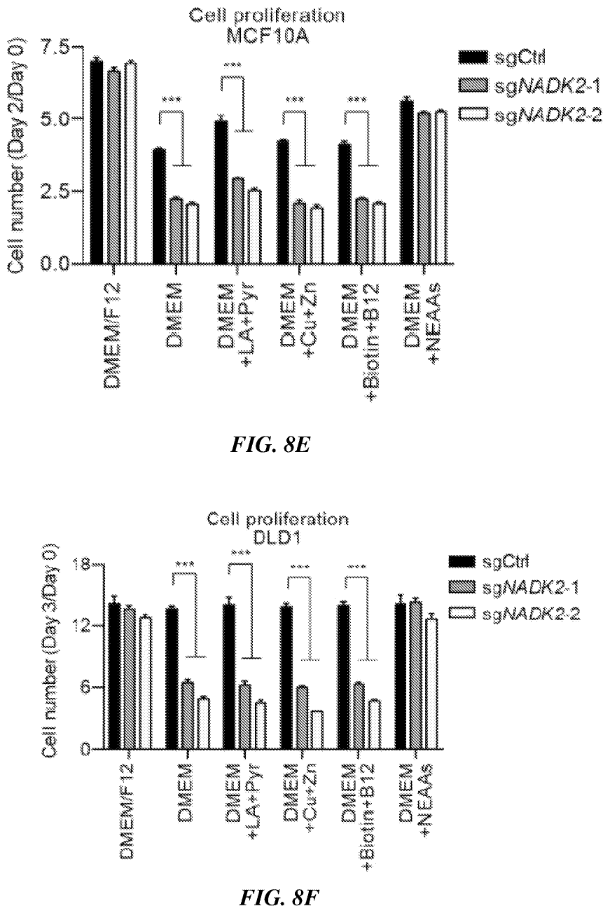

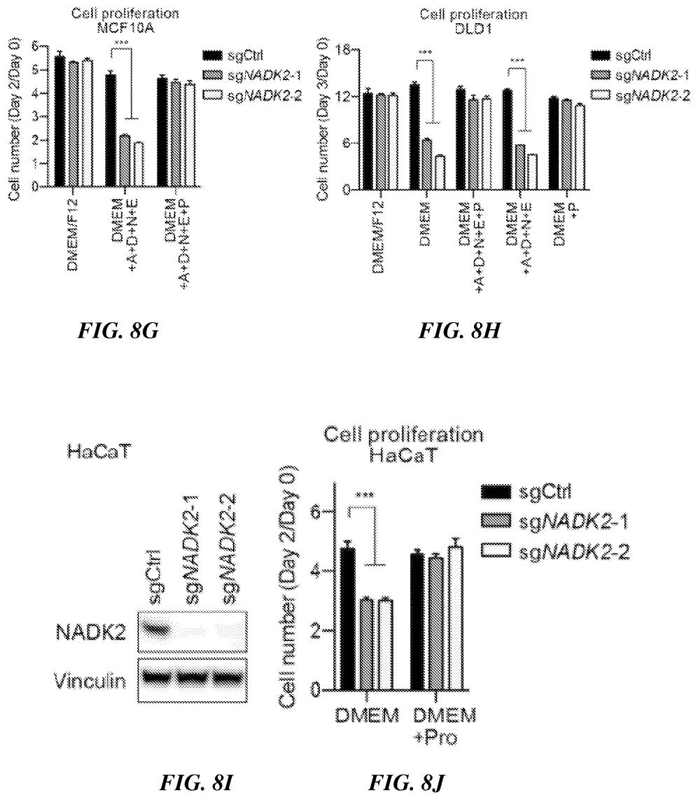

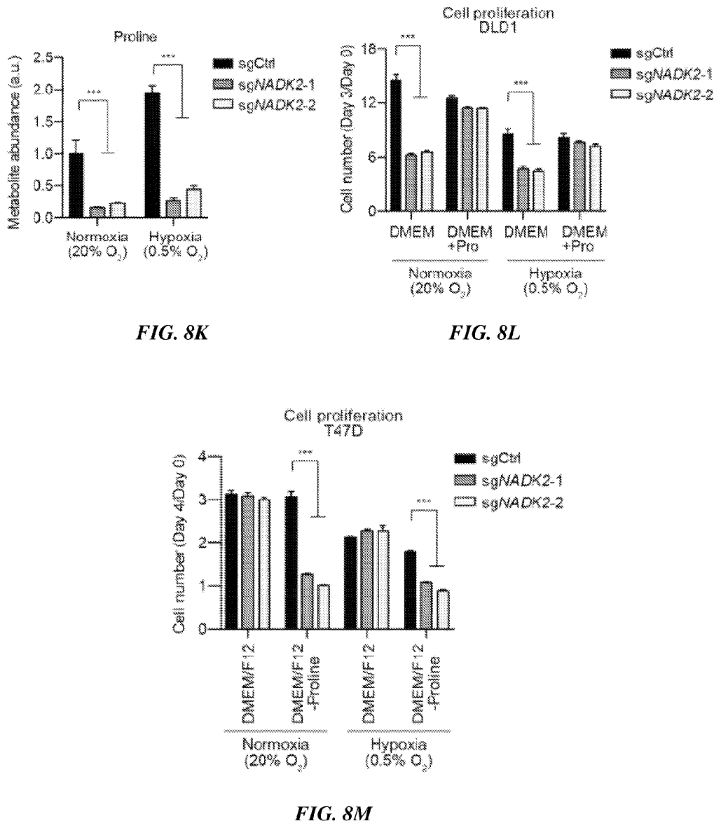

Proliferation of cells lacking NADK2 was not perturbed compared to that of control cells when cultured in a nutrient rich medium (DMEM/F12) (FIGS. 8A-8D). However, studies of IDH2-mutant cells indicated that NADK2 could have a role in NADPH-dependent biosynthesis (FIGS. 1F-1G). To test whether mitochondrial NADP(H) supports biosynthetic reactions in general, control and NADK2 knockout cells were subjected to culture medium composed of minimal essential nutrients (DMEM), and growth of NADK2-deleted cells was compromised (FIGS. 8A-8D). Apparently, mitochondrial NADP(H) promotes the synthesis of one or more nutrients required to sustain cell proliferation.

### Example 2: NADK2 is Required to Maintain Proline Biosynthesis and Collagen Deposition

The data in this Example demonstrates that NADK2 is required to maintain mitochondrial proline biosynthesis. NADK2 knock-out cells were shown to have decreased mitochondrial proline biosynthesis and decreased collagen production and deposition.

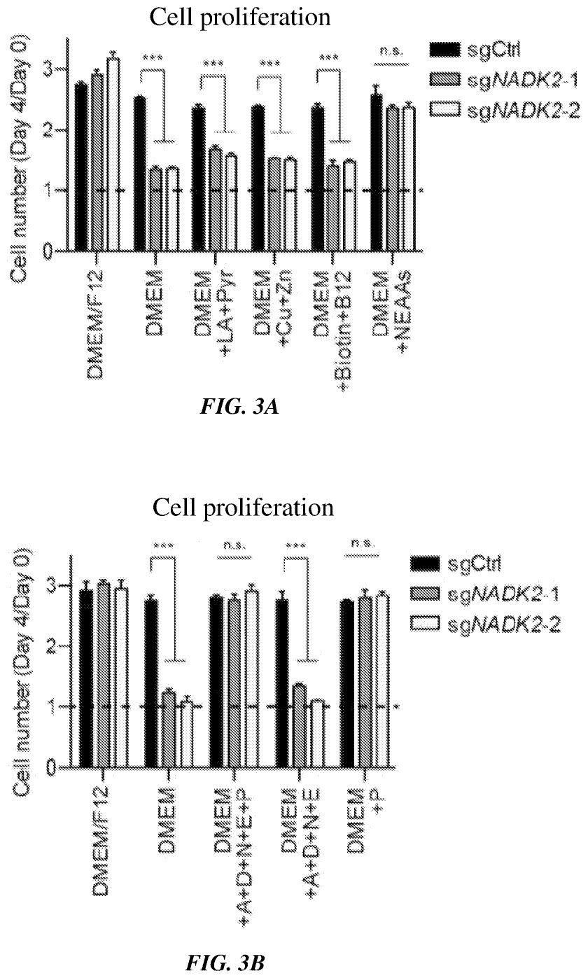

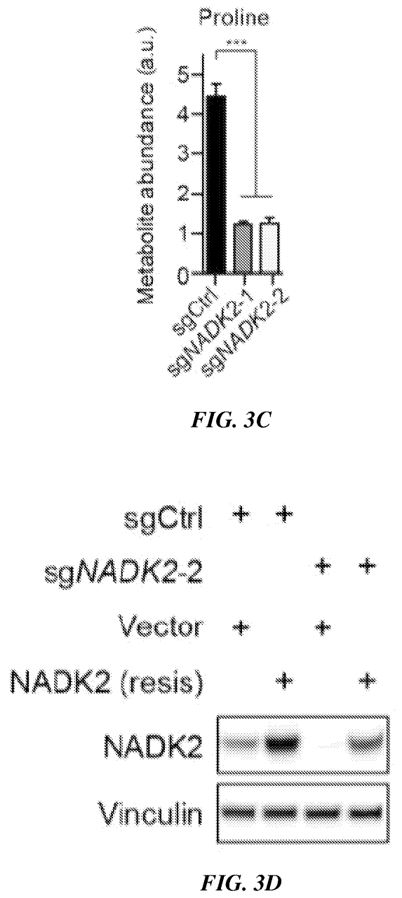

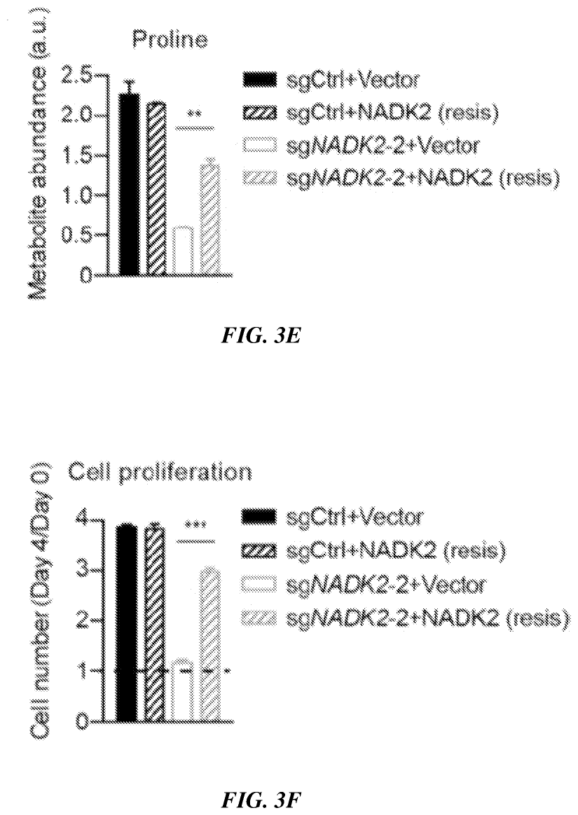

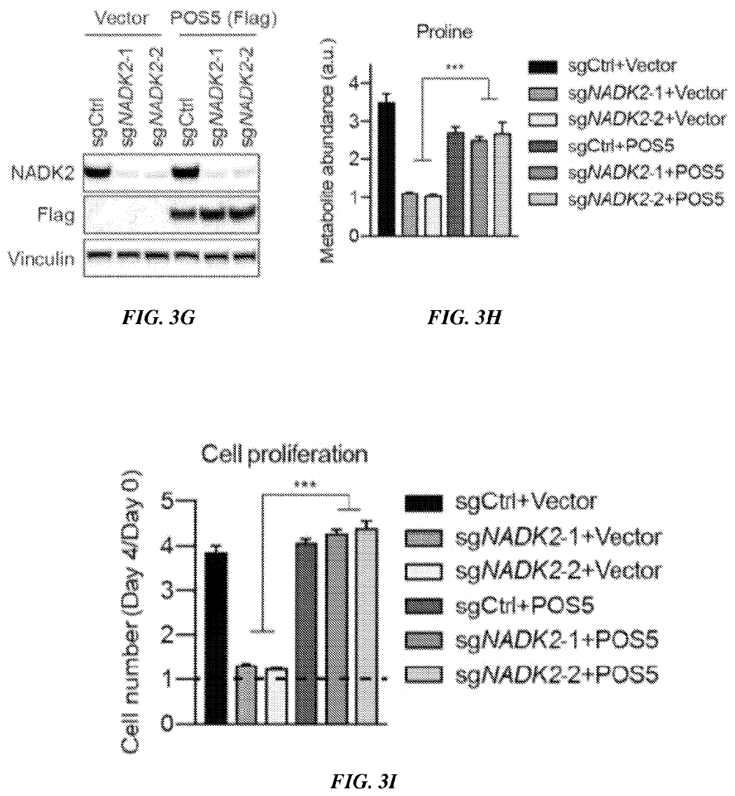

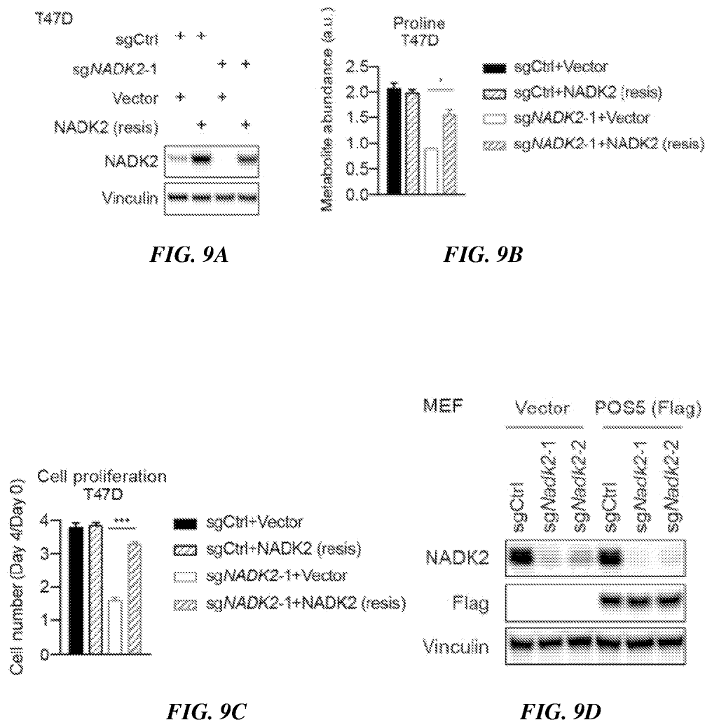

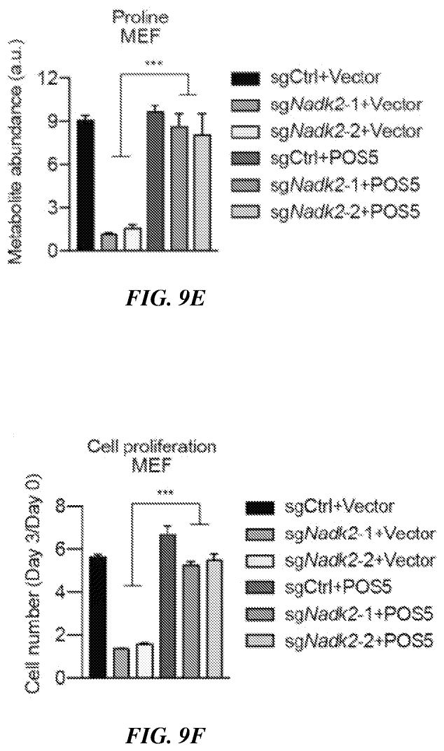

Growth of cells lacking NADK2 was restored in DMEM by supplementing non-essential amino acids (NEAAs), but not by other nutrients present in DMEM/F12 media (FIGS. 3A, 8E-8F). Supplementing individual amino acids revealed that proline was both necessary and sufficient to restore proliferation of NADK2 knockout cells in DMEM (FIGS. 3B, 8G-8J). In agreement, cells lacking NADK2 showed reduced intracellular proline abundance (FIG. 3C). Similar results were obtained when cells were maintained under hypoxia (0.5% O₂) (FIGS. 8K-8M). To validate that the proline-dependent growth phenotype was the result of NADK2 loss. NADK2 cDNA resistant to CRISPR-Cas9 mediated genome editing was introduced into the NADK2 knockout cells, which restored both intracellular proline abundance and cell growth (FIGS. 3D-3F, 9A-9C). Similar results were observed when the yeast mitochondrial NAD(H) kinase. POS5 (17), was reconstituted in NADK2-deficient cells (FIGS. 3G-3I, 9D-9F).

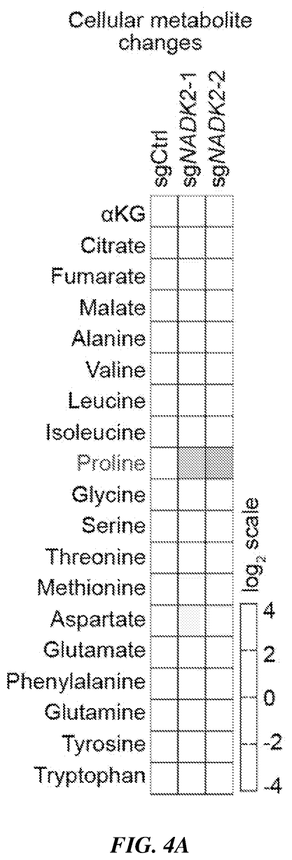

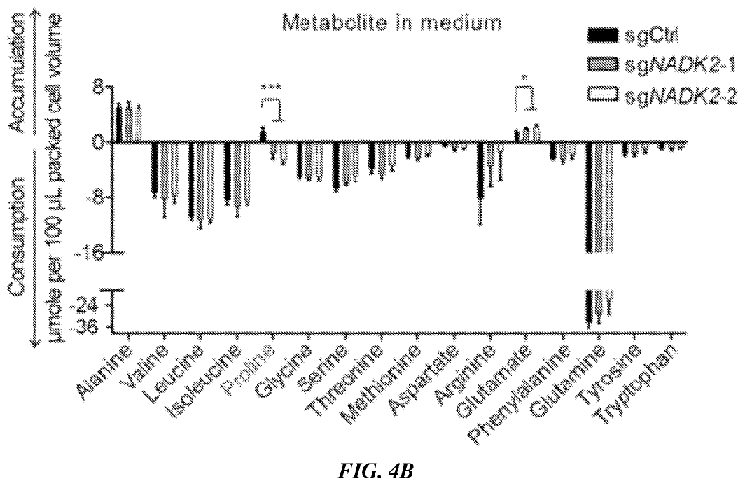

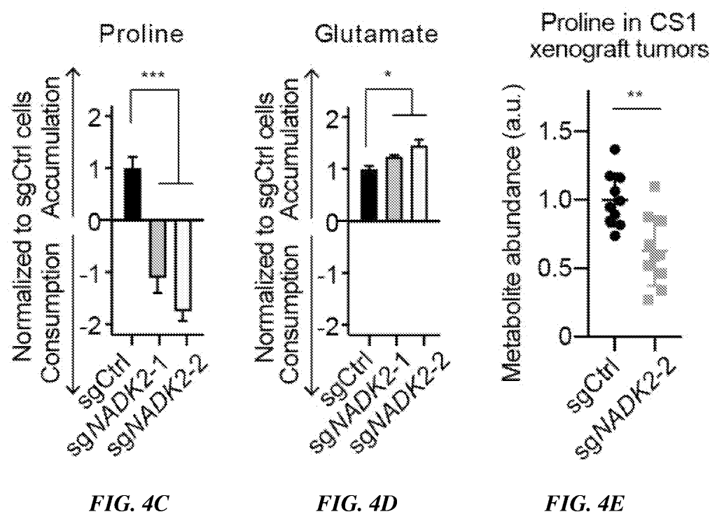

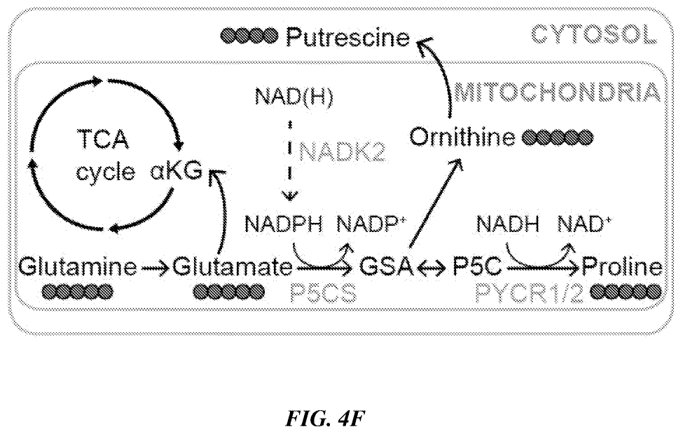

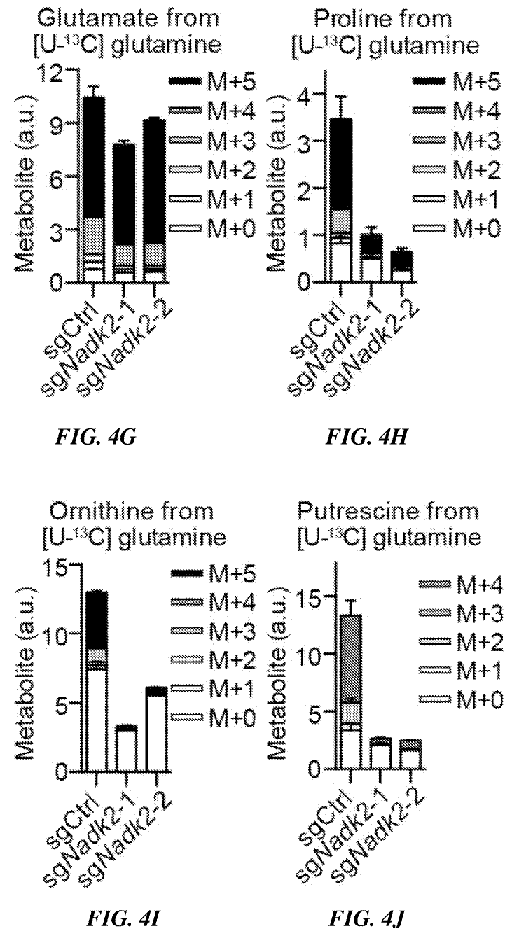

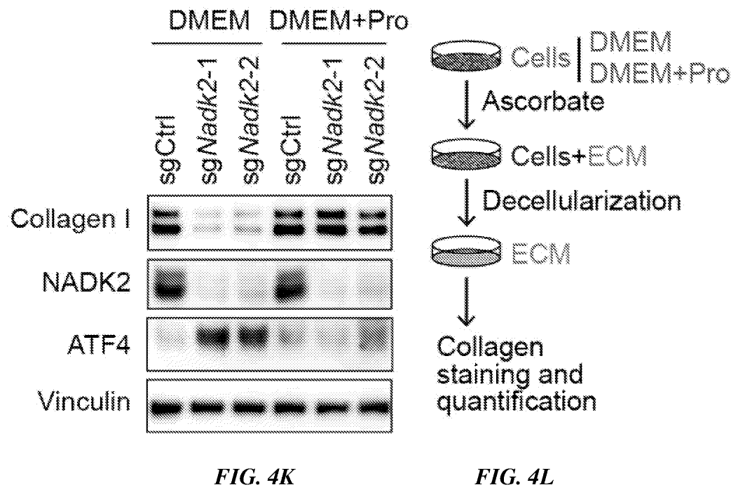

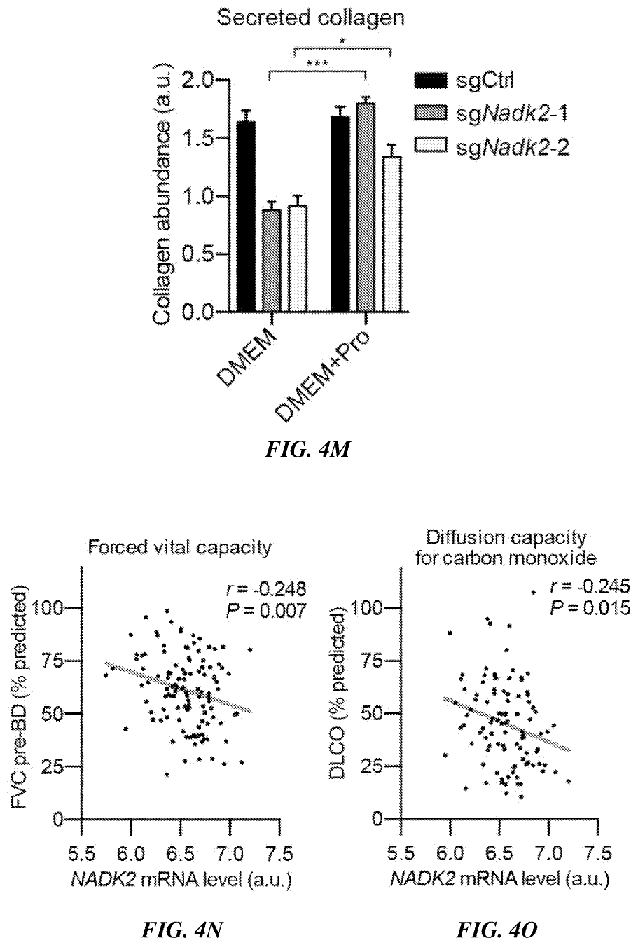

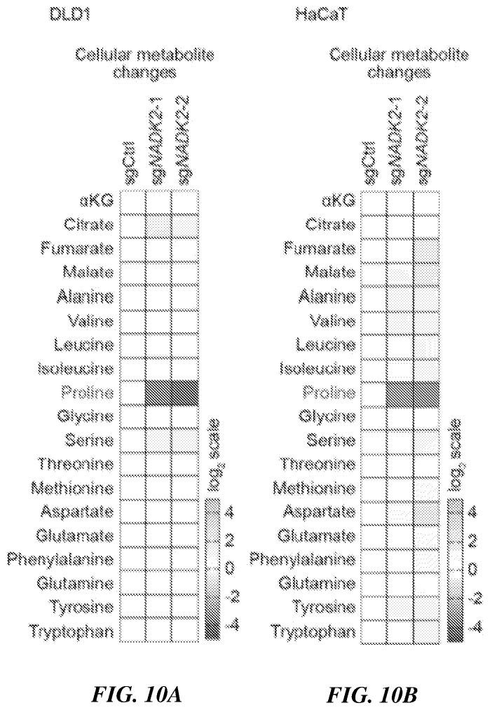

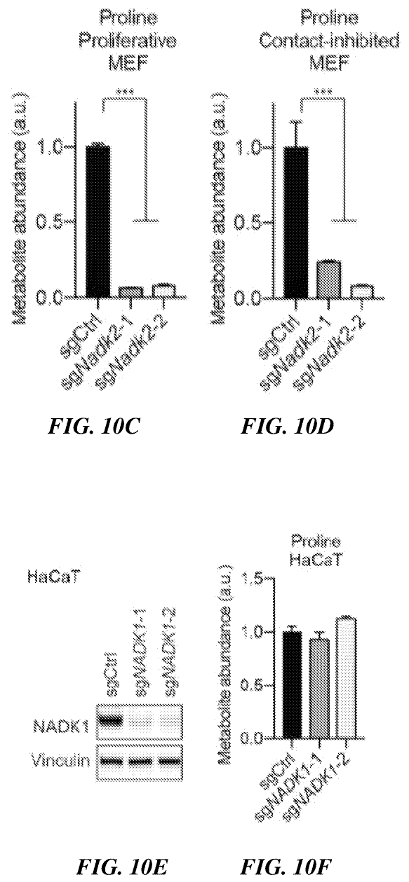

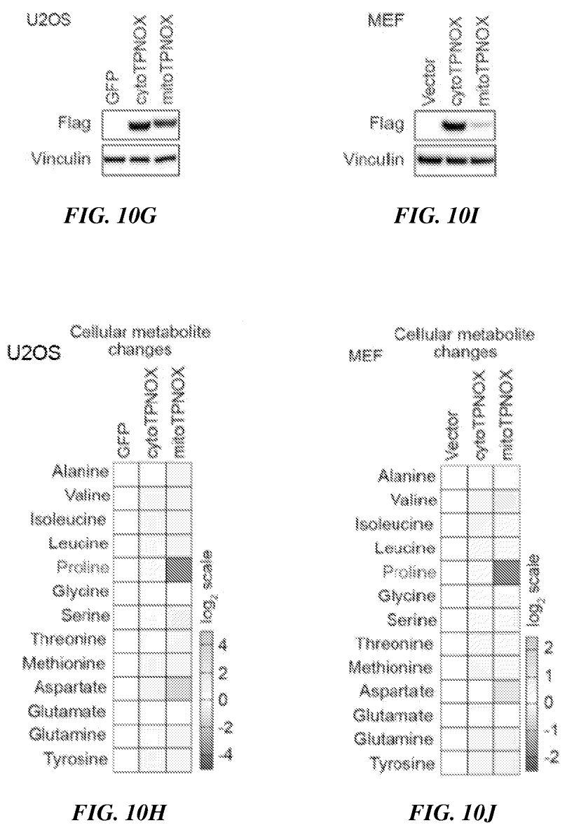

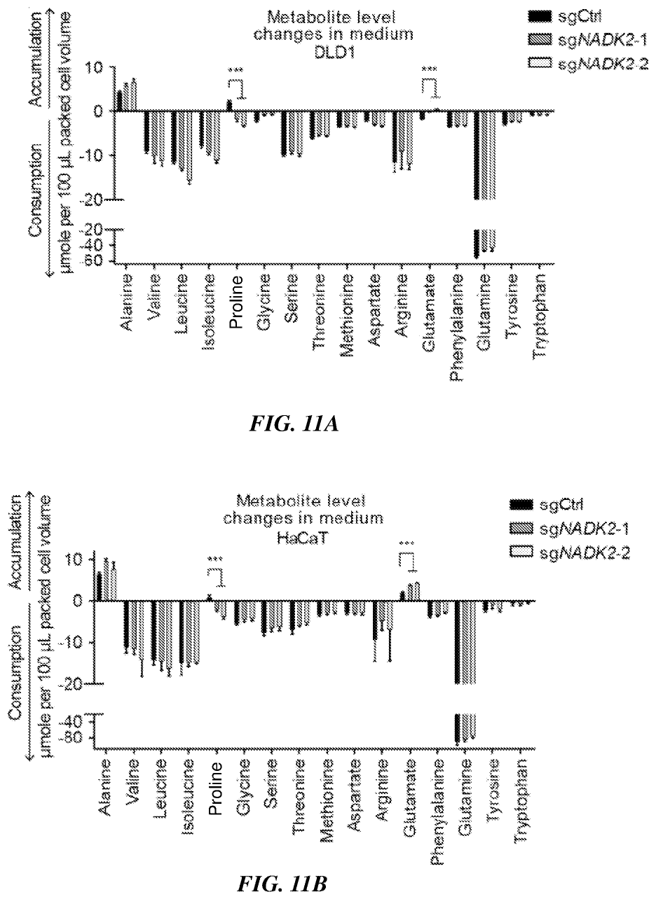

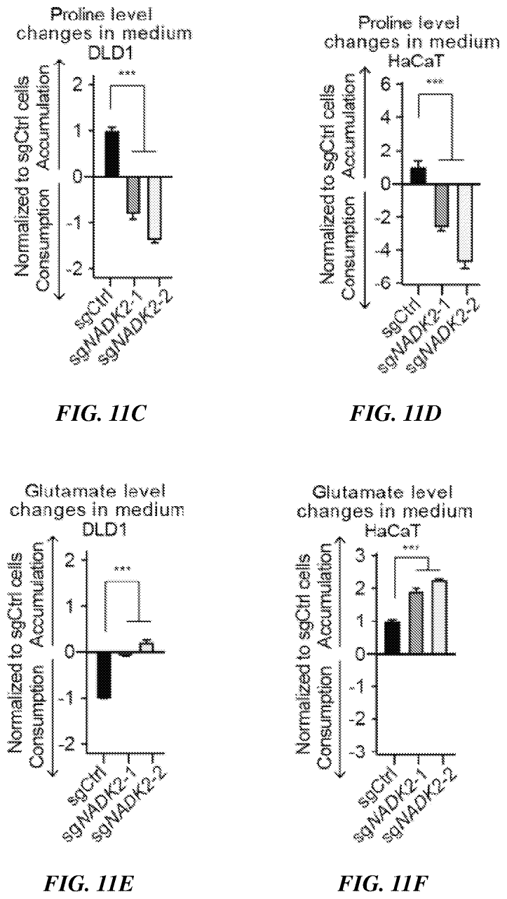

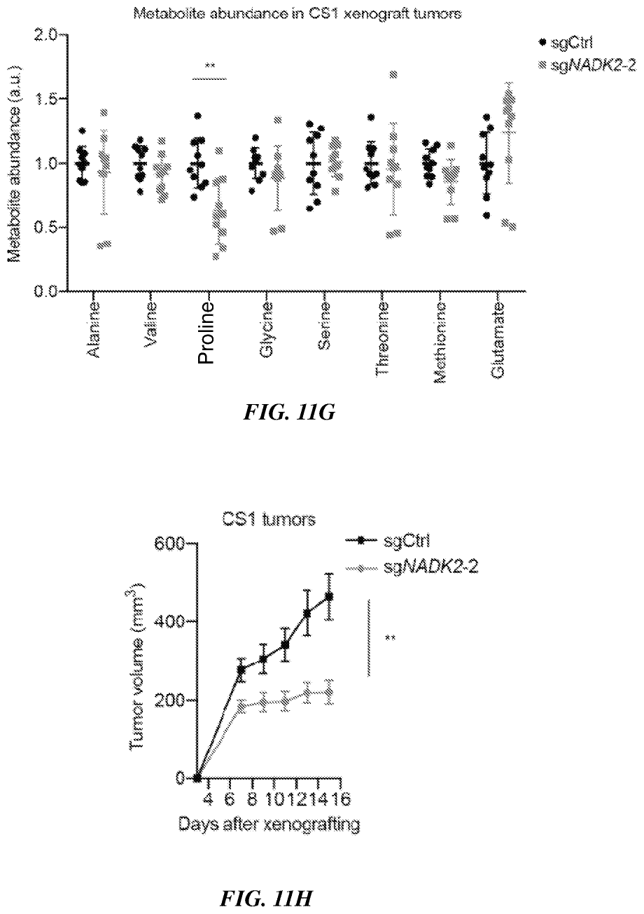

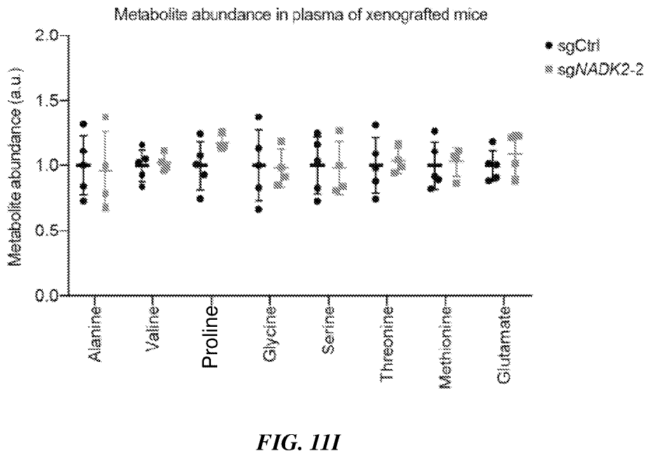

Metabolite profiling was performed on cells lacking NADK2 cultured in DMEM, and confirmed the depletion of intracellular proline, while amounts of many other amino acids were slightly increased (FIGS. 4A, 10A-10B). Loss of NADK2 also reduced proline abundance in non-proliferating (contact-inhibited) MEFs (FIGS. 10C-10D). By contrast, loss of cytosolic NADK1 did not decrease proline abundance (FIGS. 10E-10F). Likewise, the oxygen-dependent NADPH oxidase. TPNOX (18), reduced proline amounts when expressed in mitochondria (mitoTPNOX) but not in cytosol (cytoTPNOX) (FIGS. 10G-10J). To extend these observations, the consumption of nutrients from the proline-containing DMEM/F12 medium was examined. While net proline accumulation was observed in medium conditioned by control cells, proline was consumed by cells lacking NADK2 (FIGS. 4B-4C, 11A-11D). In addition, glutamate accumulation was found in medium conditioned by cells lacking NADK2 (FIGS. 4B, 4D, 11A-11B, 11E-11F), which might result from compensatory accumulation of carbon and nitrogen in the form of glutamate instead of proline. Similar analyses were performed in xenograft tumors formed by CS1 cells (FIG. 1). Proline was reduced in tumors formed by CS1 cells lacking NADK2 (FIGS. 4E, 11G), which correlated with a slower growth rate of these tumors compared to those formed by control cells (FIG. 11H). Mice grafted with control or NADK2 knockout cells displayed similar plasma levels of proline as well as other amino acids at the time of tumor resection (FIG. 11I). Thus, loss of NADK2, and the consequent depletion of mitochondrial NADP(H), results in proline auxotrophy.

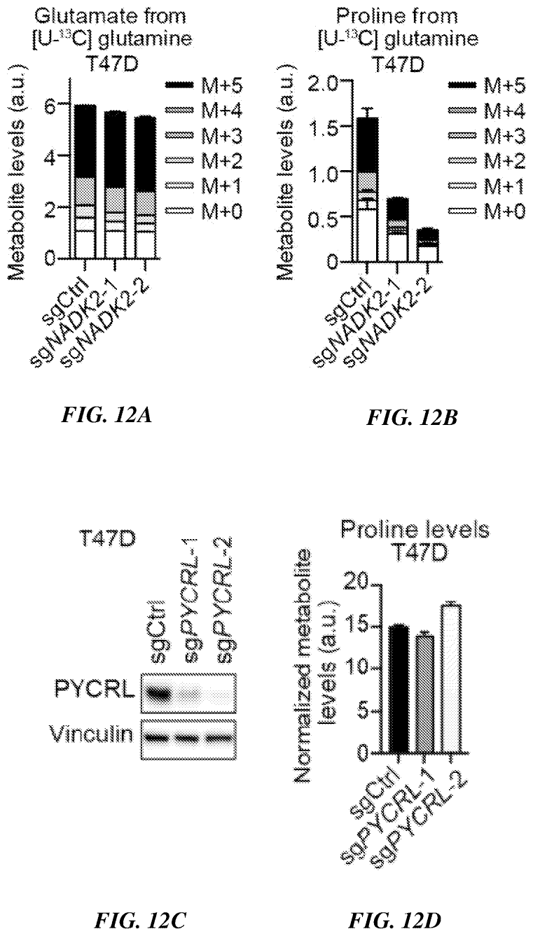

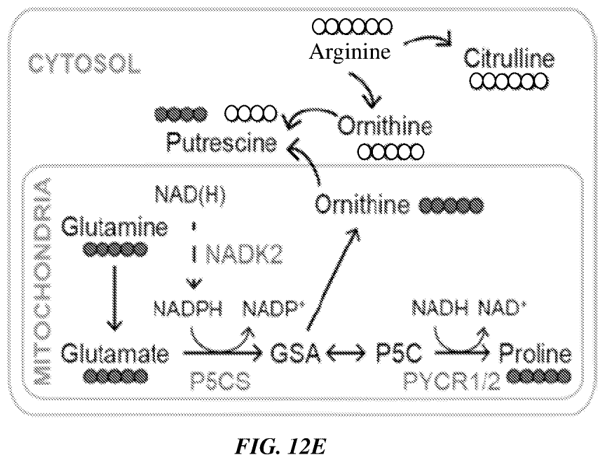

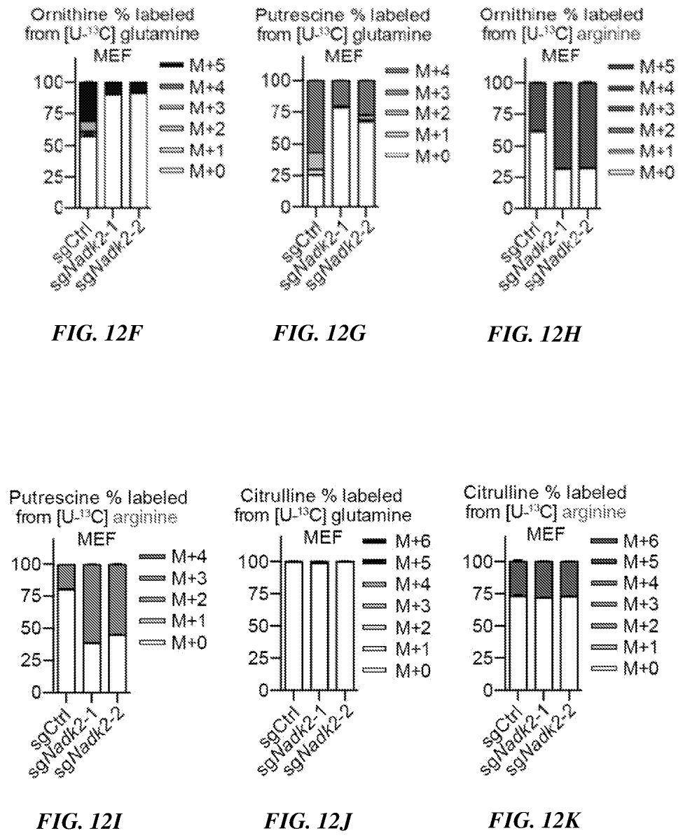

Proline biosynthesis takes place in the mitochondria, where glutamine-derived glutamate is converted to pyrroline-5-carboxylate (P5C) by pyrroline-5-carboxylate synthase (P5CS). P5C is further reduced to proline by mitochondrial pyrroline-5-carboxylate reductases (PYCR1 and PYCR2) (FIG. 4F). [U-¹³C]glutamine tracing revealed that most cellular glutamate and proline were derived from glutamine, and that glutamine-derived proline was reduced upon NADK2 loss (FIGS. 4G-4H, 12A-12B). By contrast, proline abundance was not perturbed when the cytosolic pyrroline-5-carboxylate reductase (PYCRL) was deleted (FIGS. 12C-12D).

P5CS is an NADPH-dependent enzyme, whereas PYCR1 and PYCR2 have higher affinity for NADH than for NADPH (19-21). To test if loss of NADK2 impairs conversion of glutamate to P5C by P5CS, the fact that cellular P5C is in equilibrium with glutamate-5-semialdhyde (GSA), which can be diverted to produce ornithine for polyamine biosynthesis was exploited (FIG. 4F). Intracellular arginine can also contribute to ornithine and polyamines. Isotope tracing using [U-¹³C]glutamine and [U-¹³C]arginine allowed assessing the relative contribution of these pathways to polyamine production (FIG. 12E). The fraction of ornithine and putrescine derived from [U-¹³C]glutamine decreased in cells lacking NADK2, indicating that P5CS flux from glutamate to P5C and GSA was diminished (FIGS. 4I-4J). This also resulted in a reciprocal increase in the proportional contribution of arginine to ornithine and putrescine (FIGS. 12F-12I). Because ornithine transcarbamylase expression is restricted to the liver and small intestine, loss of NADK2 did not change glutamine or arginine contribution to cellular citrulline (FIGS. 12J-12K). Thus, loss of NADK2 and the resulting decrease in mitochondrial NADP(H) blocks the reduction of glutamate to P5C required for proline biosynthesis.

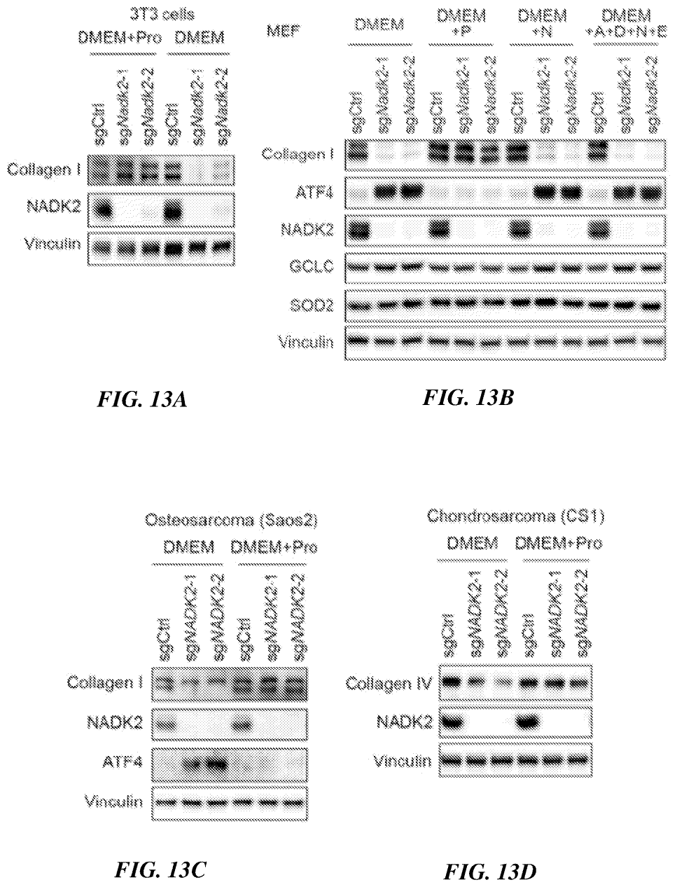

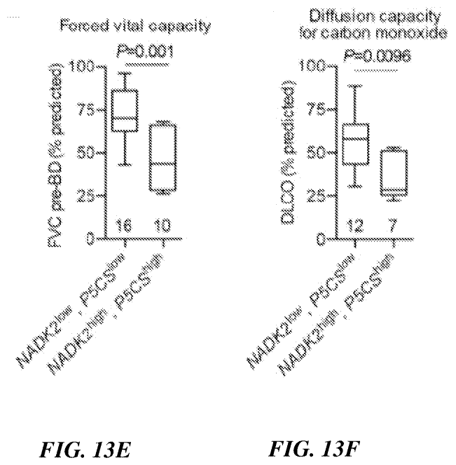

Incorporation of the proline pyrrolidine ring slows protein translation (22, 23), but endows proline-containing polypeptides with conformational rigidity. As a result, proline and its post-translationally modified form, hydroxyproline, are abundant in collagen proteins (24), so a consequence of decreased mitochondrial NADP(H) generation could be impaired collagen production. Cultured MEFs lacking NADK2 had decreased expression of collagen when grown in DMEM (FIGS. 4K, 13A). These cells accumulated activating transcription factor 4 (ATF4), indicative of amino acid shortage. Addition of 300 μM proline to the culture medium restored collagen expression and blunted ATF4 accumulation in cells lacking NADK2 (FIGS. 4K, 13A-13B). Similar results were obtained in osteosarcoma and chondrosarcoma cells that produce collagens (FIGS. 13C-13D). Fibroblasts lacking NADK2 showed decreased collagen secretion, which was rescued by proline supplementation to the medium (FIGS. 4L-4M). In patients with idiopathic pulmonary fibrosis (IPF) (25), higher NADK2 expression in the lung correlated with lower forced vital capacity (FVC) (P=0.007) and diffusion capacity for carbon monoxide (DLCO) (P=0.015), parameters that measure maximum air exhalation and the ability of lung to transfer air into the blood, respectively (FIGS. 4N-4O). Similarly, IPF patients with both high NADK2 and high P5CS expression in the lung had reduced FVC and DLCO values compared to those with low NADK2 and low P5CS expression (FIGS. 13E-13F). Thus, increased expression of NADK2 correlated with enhanced fibrotic diseases characterized by excessive collagen deposition.

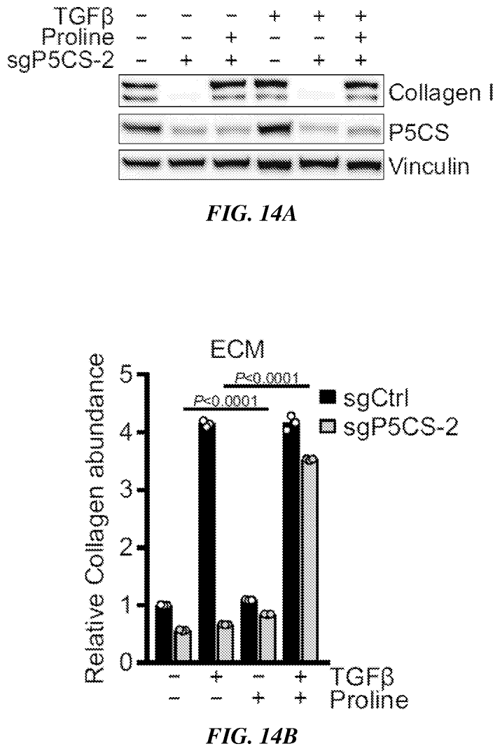

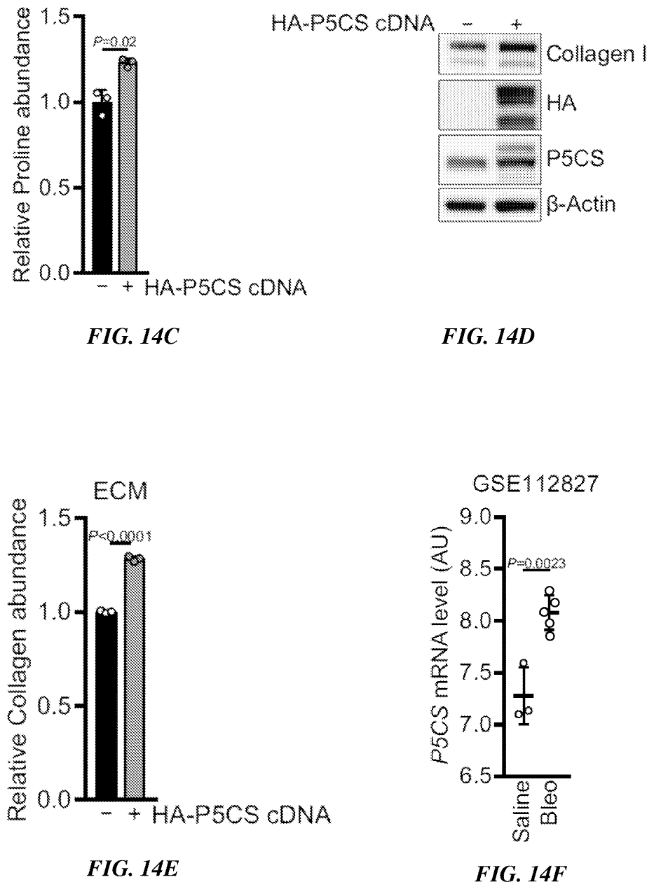

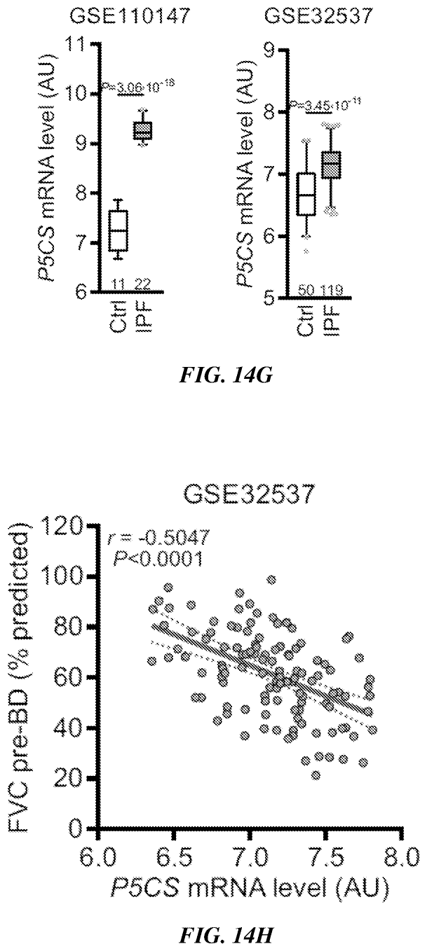

Additionally, PC5S deletion diminished expression of collagen protein both in untreated and TGFβ-treated cells, which was restored by addition of proline to the culture medium (FIG. 14A). Similar results were also obtained when measuring collagen abundance in cell-derived extracellular matrix (ECM) (FIG. 14B). Cells were genetically engineered to overexpress P5CS to test whether the upregulation of P5CS by TGFβ contributes to increased proline and collagen biosynthesis. Indeed, ectopic expression of P5CS increased the abundance of proline in cells (FIG. 14C) and elevated levels of collagen in cells and the ECM (FIGS. 14D-14E), although not to the same extent as did TGFβ stimulation. These data demonstrate that expression of P5CS is required and can be sufficient for proline and collagen biosynthesis in serum-stimulated cells growing in complete medium, and that collagen levels depend on mitochondrial proline biosynthesis.

To test whether P5CS expression could also be relevant for fibrotic diseases that are characterized by excessive collagen deposition in idiopathic pulmonary fibrosis (IPF), PC5S expression was analyzed in publicly available gene expression datasets from lungs of mice treated with bleomycin to induce pulmonary fibrosis or from lungs of IPF patients. P5CS was significantly upregulated in the bleomycin mouse model of pulmonary fibrosis (FIG. 14F) and in IPF patients compared to normal controls in two independent datasets (FIG. 14G). Moreover, the forced vital capacity (FVC) as well as the diffusing capacity for carbon monoxide (DLCO), two independent parameters of lung function, inversely correlated with expression levels of P5CS in IPF patients (FIG. 14H). Taken together, these data show that P5CS expression is critical for proline and collagen biosynthesis and correlates with disease-relevant parameters.

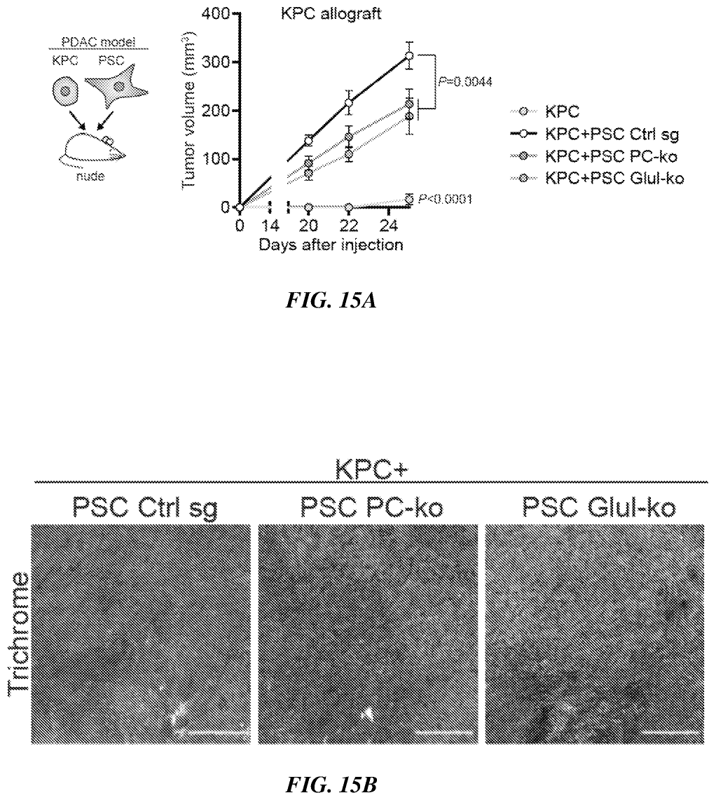

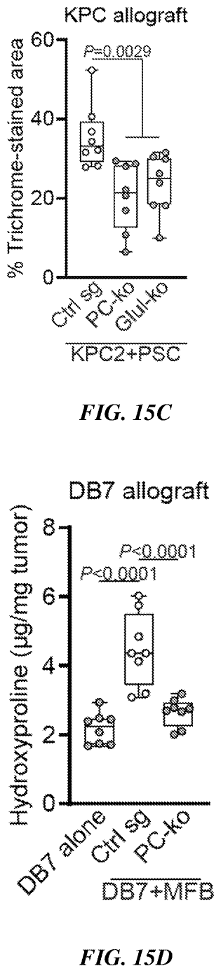

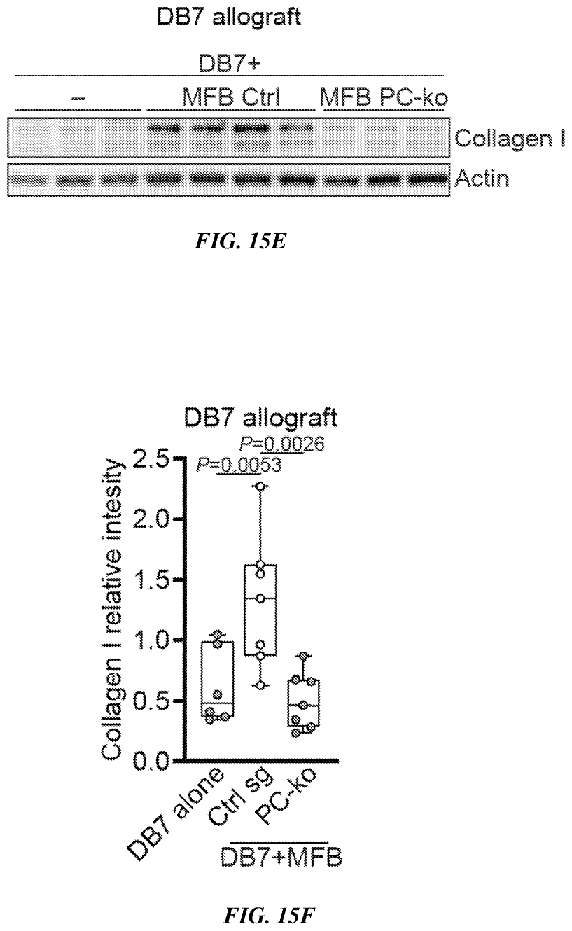

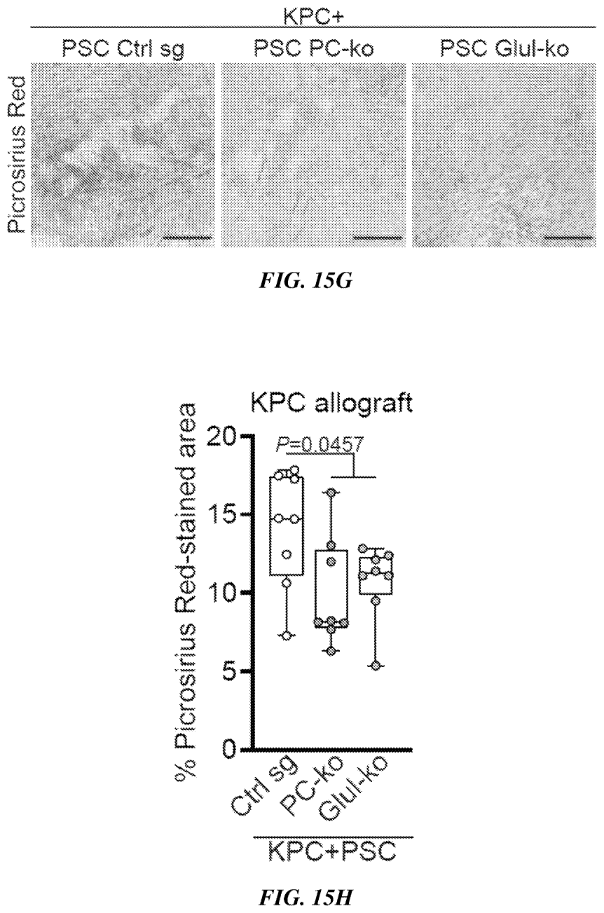

Next, the role of fibroblast pyruvate carboxylase (PC) and glutamine synthetase (GluI) in maintaining collagen levels and tumor growth in vivo was investigated. Pyruvate carboxylase converts pyruvate to oxaloacetate, a tricarboxylic acid cycle intermediate that is required to produce isocitrate, which is converted to alpha ketoglutarate (αKG) in mitochondria by IDH2. Glutamine synthetase converts glutamate to αKG in mitochondria. Low numbers of pancreatic ductal adenocarcinoma (KPC) cells were injected subcutaneously into the flanks of nude mice, cither alone or with pancreatic stellate cells (PSCs) expressing either a control, PC, or GluI single guide RNA (sgControl, sgPC, sgGluI) (FIG. 15A). The presence of PSCs promoted tumor growth substantially (FIG. 15A), as previously reported (29). While PC- or GluI-deleted PSCs retained the ability to enhance the growth of KPC-derived tumors, tumor growth was significantly reduced compared to co-injection with controls PSCs (FIG. 15A). Intratumoral fibrosis as assessed by Masson's Trichome and Picrosirius Red staining was lower in tumors formed by KPC cells that were co-injected with PC or GluI-deleted PSCs compared to control PSCs (FIGS. 15B, 15C, 15G, and 15H). Together, these data demonstrate that αKG formed by PC and glutamate synthetase are important in promoting fibrosis and tumor growth in vivo.

The ability of fibroblast pyruvate carboxylase (PC) to regulate tumor growth and collagen content was also investigated. Specifically, the possibility that the growth of DB7 murine mammary tumors could be supported by matrix proteins such as collagen secreted by primary mammary fibroblasts (MFB) was tested. Consistent with this, co-injection of MFBs substantially increased the collagen content of DB7 allograft tumors after engraftment, as measured by the levels of hydroxyproline in tumor acid hydrosylates and by Western blot (FIGS. 15D-15F). PC deletion in MFBs resulted in a more than 50% reduction of tumor collagen levels compared to co-injection of controls MFBs (FIGS. 15D-15F). Thus, fibroblast PC is required for collagen production in the tumor microenvironment.

These findings provide insights into the regulation of intracellular metabolism. In endosymbiosis with the host cell, mitochondria produce NADP(H) that supplies biosynthetic precursors to their host and appear not to use the NADP(H) for antioxidant defense in support of their own homeostasis. Compartmentalization of cellular metabolism thus has important roles in cukaryotic cells beyond the well-known collaborative production of ATP.

### Example 3: Materials and Methods and References

### Antibodies and Chemicals

Antibodies (commercial source, catalog number, detected molecular weight) used in this study were: Tubulin (Sigma, T9026, 50 kD), CS (Cell Signaling Technology, 14309, 45 kD), NADK2 (Abcam, ab181028, 45 kD), COX IV (Cell Signaling Technology, 4850T, 17 kD), Lamin A/C (Cell Signaling Technology, 4777, 75 kD and 65 kD), H3 (Abcam, ab1791, 17 kD), Vinculin (Sigma, V9131, 120 kD), CAT (Cell Signaling Technology, 12980, 60 kD), GOLGA1 (Cell Signaling Technology, 13192, 100 kD), CALR (Cell Signaling Technology, 12238, 55 kD), LAMP2 (Santa Cruz Biotechnology, sc-18822, 120 kD), CTSC (Santa Cruz Biotechnology, sc-74590, 25 kD), PRX-SO3 (Abcam, ab16830, PRX3-SO3 at 25 kD and PRX1/2-SO3 at 22 kD), PRX3 (Abcam, ab73349, 25 kD), Collagen I (Abcam, ab21286, 120 kD and 160 kD), ATF4 (Cell Signaling Technology, 11815, 47 kD), PYCRL (Thermo Fisher, MA5-25335, 30 kD), Collagen IV (Proteintech Group Inc., 55131-1-AP, 190 kD), MTHFD2 (Proteintech Group Inc., 12270-1-AP, 35 kD), SHMT2 (Cell Signaling Technology, 12762, 50 kD), Flag (Sigma, F1804, POS5-Flag at 49 kD. TPNOX-Flag at 52 kD), GCLC (Santa Cruz Biotechnology, sc-390811, 70 kD), GCLM (Proteintech Group Inc., 14241-1-AP, 30 kD), xCT (Cell Signaling Technology, 12691, 38 kD), SOD2 (Proteintech Group Inc., 24127-1-AP, 25 kD), GSR (Santa Cruz Biotechnology, sc-133245, 50 kD), NADK1 (Cell Signaling Technology, 55948, 48 kD), Cyclin D1 (Cell Signaling Technology, 55506, 35 kD).

Chemicals (commercial source, catalog number) used in this study were: Erastin (Med Chem Express, HY-15763), RSL3 (Cayman, 1219810-16-8), H2O2 (Sigma, H1009), MitoParaquat (Cayman, 18808), FK866 (Sigma, F8557), Buthionine sulfoximine (Cayman, 14484), L-alanine (Sigma, A7627), L-aspartate (Sigma, A8949), L-asparagine (Sigma, A0884), L-glutamate (Sigma, G1251), L-proline (Sigma, P0380), [U-¹³C]L-glutamine (Cambridge Isotope Laboratories, CLM-1822-H-0.25), [U-¹³C]L-arginine (Cambridge Isotope Laboratories, CLM-2265-H-0.1), [U-¹³C]glucose (Cambridge Isotope Laboratories, CLM-1396-5), [2,3,3-2H3]serine (Cambridge Isotope Laboratories, DLM-582-0.1), Lipoic acid (Sigma, T1395), Pyruvate (Life Technologies, 11360070), Biotin (Sigma, B4639), Vitamin B12 (Sigma, V6629).

### Cell Culture

The HEK293T cell line, the cancer cell lines U2OS, DLD1, T47D and Saos2, the non-malignant cell lines HaCaT and MCF10A, and the NIH-3T3 cell line were obtained from the American Type Culture Collection (ATCC). The chondrosarcoma cell lines JJ012 with an endogenous IDH1 R132G mutation and CS1 with an endogenous IDH2 R172S mutation were previously validated by sequencing the IDH1 and IDH2 genes as described (26, 27). The MEF cell line was derived by SV40 large T antigen immortalization. The MCF10A cell line was maintained in DMEM/F12 (Thermo Fisher 11320) based medium supplemented with 5% horse serum (Thermo Fisher 16050122), 20 ng/mL EGF (Peprotech, AF-100-15), 0.5 mg/mL hydrocortisone (Sigma, H0888), 100 ng/mL cholera toxin (Sigma, C8052), 10 μg/mL insulin (Sigma, 10516), and 100 unit/mL penicillin and 100 μg/mL streptomycin. Other cell lines were maintained in DMEM/F12 based medium supplemented with 10% FBS (Gemini) and 100 unit/mL penicillin and 100 μg/mL streptomycin. All cell lines were cultured in a 37° C. incubator at 20% oxygen, and were routinely verified to be mycoplasma-free by MycoAlert Mycoplasma Detection Kit (Lonza).

Contact-inhibition of MEFs was induced by seeding 125,000 cells per well in 0.1% gelatin-coated 24-well plates. Complete confluency was observed after 48 hours, and the cells were maintained for additional 96 hours, with medium change every 24 hours, before the downstream analyses.

For experiments involving nutrient and medium component manipulation, and stable isotope tracing, the denoted medium was supplemented with 10% dialyzed FBS (Gemini) and 100 unit/mL penicillin and 100 μg/mL streptomycin. The level of nutrient supplementation was determined by the amount present in DMEM/F12 medium unless otherwise specified.

### Gene Knockout and Gene Overexpression

CRISPR-Cas9 mediated gene knockout was achieved using the lentiCRISPR v2 system (Addgene 52961 and 98292), and polyclonal cell populations were used for the experiments. The human control sgRNA (sgCtrl) is targeting the silent gene PRM1 in order to achieve genome cutting, but at a non-expressed gene. Similarly, the mouse control sgRNA is targeting the ROSA26 locus. cDNA for NADK2 was obtained from Origene (RC214247), and was mutagenized to prevent targeting by guide RNA but preserve the wild-type protein sequence. cDNA for POS5 synthesized at GENEWIZ was codon optimized (see Table 2 for codon optimized POS5 cDNA) for mammalian cell expression.

TABLE 2
POS5-Flag cDNA optimized for
mammalian cell expression
Sequence

ATGTTTGTGAGGGTGAAACTGAACAAGCCCGTGAAGTGGTATAGATTCT
ACAGCACACTGGACTCCCACTCCCTCAAACTGCAGAGCGGCTCCAAGTT
CGTCAAGATCAAGCCCGTGAACAATCTGAGGAGCTCCTCCAGCGCCGAT
TTCGTGAGCCCTCCCAATTCCAAGCTCCAATCTCTGATCTGGCAGAATC
CCCTCCAGAACGTGTACATCACCAAGAAGCCTTGGACCCCCAGCACCAG
AGAAGCCATGGTGGAGTTTATCACCCATCTGCACGAGAGCTATCCCGAG
GTGAACGTCATCGTCCAGCCCGACGTGGCTGAGGAGATCAGCCAAGATT
TCAAGAGCCCCCTCGAAAACGACCCCAATAGACCCCATATTCTGTATAC
CGGCCCCGAGCAAGACATCGTCAATAGGACCGATCTGCTGGTGACACTG
GGAGGAGACGGCACCATTCTGCATGGCGTGTCCATGTTTGGCAATACCC
AAGTGCCTCCCGTGCTGGCCTTTGCTCTCGGAACACTGGGCTTTCTGCT
GCCCTTCGACTTCAAGGAGCACAAGAAGGTGTTCCAAGAGGTGATCAGC
AGCAGAGCCAAGTGCCTCCACAGAACAAGACTGGAGTGCCACCTCAAAA
AGAAGGACAGCAACTCCAGCATCGTGACCCACGCCATGAACGACATTTT
TCTGCATAGAGGCAATAGCCCCCATCTGACCAATCTGGACATCTTCATC
GATGGCGAATTTCTGACAAGGACCACCGCTGACGGCGTGGCTCTGGCTA
CACCTACCGGCTCCACCGCCTATTCTCTGTCCGCCGGCGGATCCATTGT
GAGCCCTCTGGTCCCCGCCATTCTGATGACCCCTATCTGCCCTAGGTCT
CTGTCCTTTAGACCTCTGATTCTGCCCCACTCCTCCCACATTAGAATCA
AGATCGGCAGCAAGCTCAACCAGAAACCCGTGAACTCCGTGGTCAAGCT
GTCCGTGGACGGCATCCCCCAACAAGATCTGGACGTGGGCGACGAGATT
TACGTGATCAACGAGGTGGGCACCATCTACATCGATGGCACCCAACTGC
CCACCACAAGAAAAACCGAGAACGATTTCAACAACTCCAAGAAGCCTAA
GAGGTCCGGCATTTACTGCGTGGCTAAGACAGAGAACGACTGGATCAGA
GGCATCAACGAACTGCTGGGCTTTAACAGCTCCTTCAGACTGACCAAGA
GGCAGACCGACAACGATGATTACAAGGACCACGACGGCGACTACAAGGA
TCACGACATTGATTATAAGGATGACGACGACAAGTGA
(SEQ ID NO: 1)

A FLAG tag was further fused to the C-terminus of the POS5 protein to allow antibody detection. cDNA for FLAG-tagged cytoTPNOX and mitoTPNOX were obtained from Addgene (87853 and 87854). Ectopic gene expression of cytoTPNOX and mitoTPNOX in U2OS cells was achieved through the pINDUCER20 (Addgene, 44012) tet-on viral expression system. All the other ectopic gene expression described in this study (including cytoTPNOX and mitoTPNOX in MEFs) was achieved through the pTURN-hygro-rtTA retroviral tet-on expression system. Doxycycline was used at 100 ng/mL for gene induction. The Mito-Grx1-roGFP2 and Mito-Orp1-roGFP2 constructs were obtained from Addgene (64977 and 64991). Complete antibiotic selection was applied to all genetically modified cells before proceeding to experiments. sgRNA sequences used in this study are shown in Table 3.

TABLE 3
Single guide RNA (sgRNA) sequences

| Name | Sequence | SEQ ID NO: |
|---|---|---|
| sgCtrl | GACAAAGAAGTCGCAGACGA | 2 |
| (PRM1, Human) | | |
| sgNADK2-1 | TTGAGGTTCGTCTAGTAAAG | 3 |
| sgNADK2-2 | TGATGAAGAGACTGTTCGAT | 4 |
| sgCtrl | GAAGATGGGCGGGAGTCTTC | 5 |
| (ROSA26, Mouse) | | |
| sgNadk2-1 | CAGACTTAAACCTGTCATTG | 6 |
| sgNadk2-2 | CTGCTCGAACTCGTAGCGGG | 7 |
| sgPYCRL-1 | CCAGTGTGGATGTCGACGT | 8 |
| sgPYCRL-2 | AGGCAACAAGATGGCAGCTG | 9 |
| sgMTHFD2 | TGCGGCAGGAGGTAGAAGAG | 10 |
| sgSHMT2 | TCTGAACAACAAGTACTCGG | 11 |
| sgNADK1-1 | AACTCCAGGTCTCATCGCCG | 12 |
| sgNADK1-2 | CAGCAGTAAGCAGCCGCGTC | 13 |

### Western Blot

Cells were lysed in RIPA lysis buffer (Millipore 20-188) supplemented with protease inhibitors (Thermo Fisher, 78428). Protein concentration was determined by BCA protein assay (Thermo Fisher, 23228), following which equal amount of protein was loaded and separated in polyacrylamide gels. Protein was then transferred to nitrocellulose membrane for immunoblotting.

### Subcellular Fractionation

Subcellular fraction was performed as previously described (28). Briefly, cells were washed, pelleted and lysed in cytosol extraction buffer (50 mM HEPES pH 7.4, 150 mM NaCl, 1 M hexylene glycol, 100 μM digitonin) for 10 minutes on ice. Lysates were centrifuged at 500 g for 5 min at 4° C., and supernatants were collected (cytosolic fraction) while pellets were further lysed in membrane extraction buffer (50 mM HEPES pH 7.4, 150 mM NaCl, 1 M hexylene glycol, 1% IGEPAL) and incubated at 4° C. for 10 min. Samples were then centrifuged at 3,000 g for 5 min at 4° C., and supernatants were collected (membrane fraction). Remaining pellets were resuspended in RIPA lysis buffer and incubated for 30 min at 4° C. Samples were centrifuged at 16,000 g for 15 min at 4° C., and supernatants were collected as the nuclear fraction. The same volume of extraction buffer was used for each subcellular fraction and for the whole cell lysate, such that each fraction can be compared by Western blot on the basis of equal cell number.

### Mitochondrial Immunopurification (Mito-IP)

Rapid immunopurification of mitochondria was performed following the published methodology (4). In brief, cells with control or NADK2 knockout were engineered to express the HA-tagged OMP25 protein (Addgene, 83356); or in the case of FIG. 5E, parental DLD1 cells were engineered to express the HA-tagged OMP25 protein or the Myc-tagged OMP25 protein (Addgene, 83355), 30 million cells were washed and dounce homogenized in KPBS (136 mM KCl and 10 mM KH₂PO₄, pH 7.25). The homogenate was then cleared by centrifugation and the supernatant was applied to anti-HA beads (Thermo Fisher, 88837) and incubated with rotation for 3.5 min. The resultant beads were washed with KPBS and were eluted for different downstream analyses: Triton X-100 lysis buffer (50 mM Tris-HCl, pH 7.4, 150 mM NaCl, 1 mM EDTA, 1% (vol/vol) Triton X-100, and protease inhibitors) was used to elute mitochondria for Western blot analysis; 80:20 methanol:water containing 1.5 μM ¹³C¹⁵N labeled amino acids (Cambridge Isotope Laboratories, MSK-A2-1.2) was used to elute mitochondria for liquid chromatography-mass spectrometry (LC-MS) analysis; 80:20 methanol:water was used to elute mitochondria for NAD(H) and NADP(H) measurements; glutathione lysis buffer (see below) was used to elute mitochondria for GSH measurements.

### Measurement of NAD(H) and NADP(H)

NAD(H) and NADP(H) measurements were performed using colorimetric quantification assays (Sigma, MAK037 and MAK038, respectively), with modifications as described in (6). Briefly, metabolites from whole cells or Mito-IP samples were extracted with 80:20 methanol:water. Supernatant of the extracted metabolites was dried down in a vacuum evaporator (Gene Vac EZ-2 Elite) for 2 hours. Metabolites were then resuspended in the manufacture's NADH or NADPH extraction buffer and centrifuged for 2 min at 3000 g. The supernatant was then split in half. One half was subjected to 60° C. incubation for 30 min to decompose NAD+ or NADP+. The other half was kept on ice for 30 min. 50 μL of each half of the supernatant was then transferred to a clear-bottom 96-well plate. For each assay, a series of NADH standards of 0, 1.25, 2.5, 5, 10, 20, 40, and 80 pmol/well, or NADPH standards of 0, 1.25, 2.5, 5, 10, 20, 40, and 80 pmol/well were included. 100 μL of NAD cycling buffer and enzyme mix, or NADP cycling buffer and enzyme mix (98 μL cycling buffer and 2 μL cycling enzyme mix from the manufacture) was added to each sample and incubated for 5 min to convert all NAD⁺ to NADH, or NADP⁺ to NADPH, respectively. 10 μL of manufacturer's NADH or NADPH developer was added into each well. Values were recorded with a plate reader at 450 nm at 2 hours. The amount of NADH or NADPH was calculated from the corresponding standard curves. The ice-incubated sample indicated the total abundance of NAD(H) or NADP(H), whereas the 60° C.-incubated sample indicated only the NADH or NADPH species.

### Luminescence-Based Measurement of GSH

Measurement of whole cell or mitochondrial GSH abundance or GSH/GSSG ratio was performed using GSH/GSSG-Glo assay (Promega, V6611) following the manufacture's protocol. In brief, whole cell samples were cultured in duplicate sets or Mito-IP samples were split in half following immunopurification and KPBS washes. 50 μL of total glutathione lysis reagent or oxidized glutathione lysis reagent (from the manufacture) was added to the whole cell samples, or was used to elute the Mito-IP samples. 50 μL of total glutathione lysis reagent was also added to a series of 0, 0.125, 0.25, 0.5, 1, 2, 4, and 8 μM GSH standards. After 5 min incubation at room temperature, 50 μL of luciferin generation reagent (from the manufacture) was added to each sample and incubated at room temperature for 30 min. 100 μL of luciferin detection reagent was then added to each sample. After 15 min incubation, luminescence values were measured using a Cytation 3 imaging reader. The total glutathione lysis reagent sample indicated the total abundance of GSH (both GSH and GSSG species), whereas the corresponding oxidized glutathione lysis reagent sample indicated the GSSG species.

### Metabolite Analysis Using GC-MS

For [U-¹³C]glutamine and [U-¹³C]glucose tracing studies, cells were seeded in 6-well plates, and after 40 hours transferred into medium containing 2 mM [U-¹³C]glutamine or 25 mM [U-¹³C]glucose, supplemented with 10% dialyzed FBS, and cultured for 6 hours. For other cell-based GC-MS studies, cells were seeded in 6-well plates and incubated as described in the figure legends. Metabolism was quenched by the addition of 1 mL of 80:20 methanol:water and stored at −80° C., overnight. For metabolite measurements from spent culture medium, 30 μL of cell-conditioned medium was extracted by the addition of 1 mL of 80:20 methanol:water and stored at −80° C., overnight. 30 μL of blank medium incubated for the same amount of experimental time was processed in parallel and used as a reference to determine metabolite secretion or consumption. Measured metabolite abundances were converted to approximate concentrations using the media formulation values as a reference.

The methanol-extracted metabolites were cleared by centrifugation and supernatant was dried in a vacuum evaporator (Genevac EZ-2 Elite) for 5 hours. Dried metabolites were dissolved in 40 mg/mL methoxyamine hydrochloride (Sigma, 226904) in pyridine (Thermo Fisher, TS-27530) for 90 min at 30° C., and derivatized with MSTFA with 1% TMCS (Thermo Fisher, TS-48915) for 30 min at 37° ° C. Samples were analyzed using an Agilent 7890A GC connected to an Agilent 5975C Mass Selective Detector with electron impact ionization. The GC was operated in splitless mode with constant helium gas flow at 1 mL/min. 1 μL of derivatized metabolites was injected onto an HP-5MS column, the inlet temperature was 250° C., and the GC oven temperature was ramped from 60 to 290° C., over 25 min. Peak ion chromatograms for metabolites of interest were recorded and extracted at their specific m/z with MassHunter Quantitative Analysis software v10.0 (Agilent Technologies). Ions used for quantification of metabolite levels are as follows: α-ketoglutarate m/z 304; citrate m/z 465; fumarate m/z 245; malate m/z 335; aspartate m/z 232; alanine m/z 218; glutamate m/z 363; glycine m/z 276; isoleucine m/z 260; leucine m/z 260; proline m/z 216; serine m/z 306; threonine m/z 320; tryptophan m/z 202; tyrosine m/z 354; valine m/z 218; methionine m/z 293; glutamine m/z 246; phenylalanine m/z 294; 2-hydroxyglutarate m/z 349. All peaks were manually inspected and verified relative to known spectra for each metabolite. Natural isotope abundance correction was performed using IsoCor (https://isocor.readthedocs.io/en/latest/index.html). For relative quantification, integrated peak areas were normalized to the packed cell volume of each sample.

### Metabolite Analysis Using LC-MS

For [U-¹³C]glutamine, [U-¹³C]arginine and [2,3,3-²H]serine tracing studies, cells were seeded in 6-well plates in DMEM with 150 μM proline. Cells were cultured for 40 hours and then transferred into DMEM containing 2 mM [U-¹³C]glutamine, 400 μM [U-¹³C]arginine or 400 μM [2,3,3-2H]serine, 10% dialyzed FBS, 100 unit/mL penicillin and 100 μg/mL streptomycin. Proline (150 μM) was also supplemented for [2,3,3-2H]serine tracing experiments. After 8 hours, metabolism was quenched and metabolites were extracted by aspirating medium and adding 1 mL of 80:20 methanol:water previously kept at −80° C. After overnight incubation at −80° C., cells were collected and centrifuged at 20,000 g for 20 min at 4° C. The supernatants were dried in a vacuum evaporator (Genevac EZ-2 Elite) for 3 hours. Dried extracts were resuspended in 60 μL of 60% acetonitrile in water. Samples were vortexed, incubated on ice for 20 min, and clarified by centrifugation at 20,000 g for 20 min at 4° C.

LC-MS analysis was performed with a 6545 Q-TOF mass spectrometer with Dual JetStream source (Agilent) operating in either positive or negative ionization. For positive ionization mode liquid chromatography separation was achieved on a Acquity UPLC BEH Amide column (150 mm×2.1 mm, 1.7 μm particle size, Waters). Mobile phase A was 10 mM ammonium acetate in 10:90 acetonitrile:water with 0.2% acetic acid, pH 4 and mobile phase B was 10 mM ammonium acetate in 90:10 acetonitrile:water with 0.2% acetic acid, pH 4. The gradient was 0 min, 95% B; 9 min, 70% B; 13 min, 30% B; 14 min, 30% B; 14.5 min, 95% B; 15 min, 95% B, 20 min, 95% B; 2 mins posttime. Other LC parameters were: flow rate: 400 μL/min; column temperature: 40° C., and the injection volume was 5 μL. MS parameters were: gas temp: 300° C.; gas flow: 10 L/min; nebulizer pressure: 35 psig; sheath gas temp: 350° C.; sheath gas flow: 12 L/min; VCap: 4,000 V; fragmentor: 125 V.

For negative ionization mode liquid chromatography separation was achieved on an iHILIC-(P) Classic column (100 mm×2.1 mm, 5 μm particle size, HILICON). Mobile phase A was 10 mM ammonium bicarbonate in 10:90 acetonitrile:water with 5 μM medronic acid, pH 9.4 and mobile phase B was 10 mM ammonium bicarbonate in 90:10 acetonitrile:water with 5 μM medronic acid, pH 9.4). The gradient was 0 min, 95% B; 15 min, 50% B; 18 min, 50% B; 19 min, 95% B; 19.10 min, 95% B; 25.5 min, 95% B; 2 mins posttime. Other LC parameters were: flow rate: 200 μL/min; column temperature: 40° C., and injection volume was 2 μL. MS parameters were: gas temp: 300° C.; gas flow: 10 L/min; nebulizer pressure: 40 psig; sheath gas temp: 350° C.; sheath gas flow: 12 L/min; VCap: 3,000 V; fragmentor: 125 V. Data were acquired from m/z 50-1700 with active reference masses correction (m/z: 121.05087 and 922.00980 (positive mode) or m/z: 119.03632 and 980.01638 (negative mode). Peak identification and integration were done based on in-house exact mass and retention time library built from commercial standards. Data analysis and natural isotope abundance correction were performed using MassHunter Profinder software v10.0 (Agilent Technologies).

For TTP measurements only, MS detection was performed using an Agilent 6470 triple quadrupole mass spectrometer operated in negative ionization and MRM mode. Liquid chromatography separation was using the iHILIC-(P) Classic negative method described above. MS parameters were: gas temperature 300° C.; gas flow: 10 L/min; sheath gas temperature: 350° C.; sheath gas flow: 12 L/min; VCap: 3,000 V; fragmentor: 125 V. Individual mass transitions monitored and collision energies (CE) were: TTP M+0: m/z 481.0→158.9; TTP M+1: m/z 482.0→158.9; TTP M+2: m/z 483.0→158.9. For all transitions, collision energy was 32 V, cell accelerator voltage is 4 V. Potentially confounding signals from UTP and CTP were also monitored and chromatographic separation confirmed so they did not interfere with TTP measurements. Data analysis was using MassHunter Quantitative Analysis software v10.0 (Agilent Technologies) and natural isotope abundance correction was performed using IsoCorrectoR (https://github.com/chkohler/IsoCorrectoR).

For metabolomic profiling of the Mito-IP samples, dried extracts were resuspended in 30 μL of 60:40 acetonitrile:water and an additional 7.5 μL of 100% methanol added to prevent phase separation. Samples were vortexed, incubated on ice for 20 min, and clarified by centrifugation at 20,000 g for 20 min at 4° C. LC-MS analysis was using the iHILIC-(P) Classic negative method described with 6545 Q-TOF mass spectrometer (Agilent Technologies). Whole cell extracts were analyzed in parallel and data analysis was performed using MassHunter Profinder v10.0 software (Agilent Technologies). Metabolite identifications reported were based on either (a) exact mass and retention times matched to authentic standards (denoted as RT in Tables 1A-1G) or (b) exact mass and MS2 spectra match using SIRIUS software (denoted as MS2 in Tables 1A-1G) (https://bio.informatik.uni-jena.de/software/sirius/). Metabolites were considered to be mitochondrial if the average peak area measured in anti-HA Mito-IPs from HA-tagged OMP25 cells was at least 1.5-fold more than in anti-HA Mito-IPs from the control cell expressing Myc-tagged OMP25 (scc Tables 1A-1G; FC>1.5 for [OMP25HA sgCtrl MitoIP vs. OMP25Myc MitoIP]). Outlier identification and exclusion were performed with Grubbs' test (α=0.01) for data shown in FIGS. 5F-5G.

### Measurement of Oxygen Consumption Rate

Oxygen consumption rate (OCR) was measured using a XFe96 Extracellular Flux Analyzer (Agilent). Cells were plated in Seahorse microplates (Agilent) at appropriate densities (10,000 cells/well for DLD1 and HaCaT cells, or 6,000 cells/well for MEFs), and were allowed to adhere overnight. Cell culture media were then removed and replaced with Seahorse media (DMEM containing 10 mM glucose, 2 mM glutamine, and 1 mM sodium pyruvate). OCR analysis was performed at basal level and after subsequent injections of oligomycin (2 μM), FCCP (0.5 μM), and rotenone plus antimycin mix (both 0.5 μM) according to the manufacturer's instructions. Immediately after OCR measurement, cell number and volume were determined using a Multisizer 3 Coulter Counter (Beckman). OCR results were analyzed using the Wave software (Agilent) under default settings and were normalized to packed cell volume.

### Reactive Oxygen Species (ROS) Measurement

Cellular ROS levels were measured by the CM-H2DCFDA oxidative stress indicator (Thermo Fisher, C6827) following the recommended manuals. Briefly, cells were incubated with 1 μM CM-H2DCFDA at 37° ° C. for 30 minutes. Cells were then harvested, and fluorescence signals were determined by flow cytometry.

### Cell Death Quantification

Cells were seeded in 96-well plates at appropriate cell densities (DLD1: 10000 cells/well, T47D: 15000 cells/well), and incubated overnight at 37° C. containing 5% CO₂. Contact-inhibited MEFs were seeded in 24-well plates and incubated as described above. Cell were then subjected to treatments as described in figures. Cells were stained with Hoechst 33342 (0.1 μg/ml) to monitor total cell number, and with Sytox Green (5 nM) to monitor cell death. Culture plates were read by Cytation 5 at indicated time point. Percentage of cell death was calculated as Sytox Green-positive cell number over total cell number.

### Mitochondrial Superoxide Measurement

Mitochondrial superoxide levels were measured by the MitoSox indicator (Thermo Fisher, M36008) following the recommended manuals. Briefly, mock or rotenone (Cayman, 13995) treated cells were incubated with 2.5 μM MitoSox reagent in HBSS (Thermo Fisher, 24020117) at 37 ºC for 10 minutes. Cells were then harvested, and fluorescence signals were determined by flow cytometry.

Mitochondrial H₂O₂ and Mitochondrial Glutathione Oxidation Measurement

Cells expressing Mito-Orp1-roGFP2 were treated with vehicle (DMSO) or MitoParaquat (100 μM) (MitoPQ, Cayman, 18808) for 24 hours. Cells expressing Mito-Grx1-roGFP2 were mock treated or treated with H₂O₂ (100 μM) (Sigma, H1009) for 4 hours. Cells were washed and incubated with 20 mM N-ethylmaleimide (NEM, Sigma, E3876) for 5 min to prevent further probe oxidation. Cells were harvested, fixed with 4% formaldehyde, and analyzed by flow cytometry using a 520/10-nm filter. The ratio of emission after excitation at 405 and 488 nm was calculated as a measure of mitochondrial H₂O₂ abundance (Mito-Orp1-roGFP2) or glutathione oxidation (Mito-Grx1-roGFP2). The maximal oxidized and reduced form of the probe was determined for each experiment by incubating cells in extra wells with 5 mM H₂O₂ or 10 mM DTT (Thermo Fisher, R0861) for 5 min before adding NEM. Oxidation status was expressed as percentage of maximal oxidized form of the probe.

### Extracellular Matrix Extraction and Collagen Staining

Extracellular matrix (ECM) extraction and collagen staining were performed as previously described (24). In brief, confluent MEFs were grown for two days on plates coated with 0.1% gelatin in the presence of 50 μM ascorbate (Sigma, A4034) in the indicated medium. Plates were decellularized with 20 mM ammonium hydroxide/0.5% Triton X-100 for 5 min on a rotating platform. Three times the volume of PBS was added, and ECM was equilibrated overnight at 4° C., followed by four additional PBS washes. To measure collagen abundance, extracted ECM was stained with the Picro Sirius Red Stain Kit (Abcam, ab150681) according to the manufacturer's instructions. The stain was extracted with 0.1 M NaOH, and optical density was measured at 550 nm using a microplate reader. Values were normalized to the packed cell volume of cells grown on a separate plate under the same experimental conditions.

### Tumor Xenograft Assay

Female nude mice (Mus musculus, Athymic Nude-Foxn1nu, Envigo 069) between the ages of 7 to 9 weeks old were used for the tumor xenograft experiment. 10 mice were randomly assigned into two groups (5 mice per group). 8 million CS1 cells with control or NADK2 knockout were implanted subcutaneously per flank on both flanks of each mouse. Tumor size was measured by calipers every other day starting from Day 7 post implantation. Measurements were taken in two dimensions, and tumor volume was calculated as length×width2×π/6. On Day 15 post implantation, all tumors were collected and snap-frozen in liquid nitrogen. Metabolites from powdered tumors were extracted using 40:40:20 acetonitrile:methanol:water (20 μL/mg of powdered tumor). Samples were sonicated, vortexed, and subjected to 2 freeze-thaw cycles, then centrifuged at 20,000 g for 20 min at 4° C., and an equal volume of supernatant was dried in a vacuum evaporator for 2 hours. At the time of tumor collection, blood was taken from each of the mice by retro-orbital bleeding and was immediately placed in EDTA-tubes. Blood samples were then centrifuged at 850 g for 10 min at 4° C. to separate plasma. 25 μL of plasma from each sample was taken. Metabolism was quenched and metabolites were extracted by addition of 1 mL of 80:20 methanol:water and kept at −80° C., overnight. Extracted metabolites were centrifuged at 20,000 g for 20 min at 4° C., and supernatant was dried in a vacuum evaporator for 2 hours. GC-MS was performed to examine metabolites in tumors and in plasma samples. Animal experiments described adhered to policies and practices approved by the Memorial Sloan Kettering Cancer Center Institutional Biosafety Committee (IBC) and the Institutional Animal Care and Use Committee (IACUC).

### Analysis of Gene Expression Datasets and Patient Data

Analysis of gene expression and patient data was performed as previously described (24). Briefly, processed gene expression dataset GSE32537 was downloaded from Gene Expression Omnibus (GEO) with GEOquery package and assigned to groups in R studio v3.6.1 (www.r-project.org). Available clinical data for GSE32537 was correlated to NADK2 gene expression using Pearson correlation analysis. Patients were grouped into low- or high-expressers according to the gene expression of P5CS or NADK2 being within the first (low) or forth quartile (high) of the gene expression range. Data was then filtered for values being present in both the P5CS^{high} and NADK2^{high} group, or the P5CS^{LOW} and NADK2^{low} group.

### Spheroid Outgrowth

Spheroids were generated by plating 1×104 KPC cells in ultra-low attachment spheroid microplates (Corning). The next day, spheroids were transferred to 24-well plates containing synthetic ECM or fibroblast-derived ECM using a P1000 pipette at one spheroid per well. Synthetic ECM was generated by gelating different concentrations of high-concentration rat tail collagen I (Corning) and growth-factor reduced Matrigel (Corning) at a final concentration of 20% in a 37° C. incubator for 1 h. Spheroids were cultured on top of ECM in DMEM with 10% FBS and were imaged 2-3 h after transfer on ECM (d0) and the three following days with a Zeiss AxioCam microscope. Spheroid area, including outgrowing cells, was quantified manually in Fiji.

### Measurement of Hydroxyproline Levels in Tumors

Flash frozen tumors were ground to a powder in a cryocup grinder (BioSpec) cooled with liquid nitrogen. Acid hydrolysates were generated from aliquots of 5-10 mg ground tumor by addition of 6 N HCl (100 μL/mg) and incubation at 95° C. for 16 h. Samples were cooled to room temperature and centrifuged at 20,000 g for 10 min. 100 μL supernatant was dried in a vacuum evaporator (Genevac EZ-2 Elite) for 2 h, and hydroxyproline levels were measured by GC-MS as described below.

### Mass-Spectrometry Measurement of TCA Cycle Metabolites and Amino Acids

GC-MS measurements were performed as described before (30). Ions used for quantification of metabolite levels were as follows: d5-2HG m/z 354; citrate m/z 465; alpha-ketoglutarate m/z 304; succinate m/z 247; fumarate m/z 245; malate m/z 335; aspartate m/z 232; hydroxyproline m/z 332; proline m/z 216; glutamate m/z 246; glutamine m/z 245; lactate m/z 219; pyruvate m/z 174. All peaks were manually inspected and verified relative to known spectra for each metabolite. For relative quantification of cell samples, integrated peak areas were normalized to the internal standard d5-2HG and to the packed cell volume of each sample. Absolute quantification of hydroxyproline in tumor acid hydrolysates was performed against a standard curve of commercial trans-4-hydroxy-L-proline (Sigma). In stable isotope tracing experiments, natural isotope abundance correction was performed with IsoCor software (30). LC-MS measurements were performed as described before (30). Peak identification and integration were done based on exact mass and retention time match to commercial standards. Data analysis and natural isotope abundance correction were performed with MassHunter Profinder software v10.0 (Agilent Technologies).

### Tumor Allograft Experiments

For the pancreatic ductal adenocarcinoma (PDAC) allograft model, 1×10⁵ KPC cells alone or together with 5×10⁵ PSCs were resuspended in 100 μL PBS and injected subcutaneously into the flanks of 8-10 weeks old female athymic Nude-Foxn1^{nu} mice (Envigo, 069). For the BRCA allograft model, 5×10⁵ DB7 cells alone or together with 5×10⁵ MFBs were resuspended in 100 μL PBS and injected subcutaneously into the flanks of 8-10 weeks old female FVB/N mice (JAX, 001800). In one experiment, 5×10⁵ DB7 cells were injected in 1:1 of 100 μL Matrigel (Corning) and PBS. At the beginning of each experiment, mice were randomly assigned to the different groups. No estimation of sample size was performed before the experiments. Mice were monitored daily and tumor volume was measured by calipers. Measurements were carried out blindly by members of the MSKCC Antitumor Assessment Core and were taken in two dimensions, and tumor volume was calculated as length×width2×π/6. At the end of the experiment, mice were euthanized with CO₂, and tumors were collected and aliquoted for 10% formalin fixation and/or snap freezing.

### Histology

Tissues were fixed overnight in 10% formalin, dehydrated in ethanol, embedded in paraffin, and cut into 5 μm sections. Picrosirius Red staining was performed with the Picro Sirius Red Stain Kit (Abcam) according to the manufacturer's instructions. Masson's trichrome staining was performed with the Masson's Trichrome Stain Kit (Polysciences) according to the manufacturer's instructions. For immunofluorescence staining, sections were de-paraffinized with Histo-Clear II (National Diagnostics) and rehydrated. Antigen retrieval was performed for 40 min in citrate buffer pH 6.0 (Vector Laboratories) in a steamer (IHC World). Sections were blocked in 5% BSA and 5% normal goat serum (Cell Signaling) in TBS containing 0.1% Tween-20, and incubated in primary antibodies at 4° C. in a humidified chamber overnight. Sections were incubated in secondary antibody in blocking solution for 1 h at room temperature and mounted in Vectashield Vibrance Antifade Mounting Medium with DAPI (Vector Laboratories). The following primary antibodies were used: SMA (Millipore, CBL171), CK8 (DSHB, TROMA-I). The following secondary antibodies were used: donkey anti-mouse Alexa-Fluor 488, donkey anti-rat Alexa Fluor 647 (Thermo Scientific).

### LIST OF CITED REFERENCES

1. R. P. Goodman, S. E. Calvo, V. K. Mootha, Spatiotemporal compartmentalization of hepatic NADH and NADPH metabolism. The Journal of biological chemistry 293, 7508-7516 (2018).
2. C. A. Lewis et al., Tracing compartmentalized NADPH metabolism in the cytosol and mitochondria of mammalian cells. Molecular cell 55, 253-263 (2014).
3. K. Ohashi, S. Kawai, K. Murata, Identification and characterization of a human mitochondrial NAD kinase. Nature communications 3, 1248 (2012).
4. W. W. Chen, E. Freinkman, D. M. Sabatini, Rapid immunopurification of mitochondria for metabolite profiling and absolute quantification of matrix metabolites. Nature protocols 12, 2215-2231 (2017).
5. W. W. Chen, E. Freinkman, T. Wang. K. Birsoy, D. M. Sabatini, Absolute Quantification of Matrix Metabolites Reveals the Dynamics of Mitochondrial Metabolism. Cell 166, 1324-1337 e1311 (2016).
6. G. Hoxhaj et al., Direct stimulation of NADP(+) synthesis through Akt-mediated phosphorylation of NAD kinase. Science 363, 1088-1092 (2019).
7. C.-K. C. Ding et al., MESH1 is a cytosolic NADPH phosphatase that regulates ferroptosis. Nature Metabolism 2, 270-277 (2020).
8. A. H. Shih, O. Abdel-Wahab, J. P. Patel, R. L. Levine, The role of mutations in epigenetic regulators in myeloid malignancies. Nature reviews. Cancer 12, 599-612 (2012).
9. G. S. Ducker et al., Reversal of Cytosolic One-Carbon Flux Compensates for Loss of the Mitochondrial Folate Pathway (vol 23, pg 1140, 2016). Cell metabolism 24, 640-641 (2016).
10. N. Kory et al., SFXN1 is a mitochondrial serine transporter required for one-carbon metabolism. Science 362, 791-+(2018).
11. H. Kong, N. S. Chandel, Regulation of redox balance in cancer and T cells. The Journal of biological chemistry 293, 7499-7507 (2018).
12. M. Gutscher et al., Real-time imaging of the intracellular glutathione redox potential. Nature methods 5, 553-559 (2008).
13. M. Gutscher et al., Proximity-based protein thiol oxidation by H2O2-scavenging peroxidases. The Journal of biological chemistry 284, 31532-31540 (2009).
14. E. L. Robb et al., Selective superoxide generation within mitochondria by the targeted redox cycler MitoParaquat. Free Radic Biol Med 89, 883-894 (2015).
15. M. Gao et al., Role of Mitochondria in Ferroptosis. Molecular cell 73, 354-363 e353 (2019).
16. B. R. Stockwell et al., Ferroptosis: A Regulated Cell Death Nexus Linking Metabolism, Redox Biology, and Disease. Cell 171, 273-285 (2017).
17. C. E. Outten, V. C. Culotta, A novel NADH kinase is the mitochondrial source of NADPH in Saccharomyces cerevisiae. Embo Journal 22, 2015-2024 (2003).
18. V. Cracan, D. V. Titov, H. Y. Shen, Z. Grabarek, V. K. Mootha, A genetically encoded tool for manipulation of NADP(+)/NADPH in living cells. Nat Chem Biol 13, 1088-+(2017).
19. J. De Ingeniis et al., Functional specialization in proline biosynthesis of melanoma. PloS one 7, e45190 (2012).
20. J. J. Kramer, R. C. Gooding, M. E. Jones, A radiochemical assay for a NADP+-specific gamma-glutamate semialdehyde dehydrogenase extracted from mitochondrial membrane of rat intestinal epithelial cells. Analytical biochemistry 168, 380-386 (1988).
21. J. M. Phang, Proline Metabolism in Cell Regulation and Cancer Biology: Recent Advances and Hypotheses. Antioxidants & redox signaling 30, 635-649 (2019).
22. M. Y. Pavlov et al., Slow peptide bond formation by proline and other N-alkylamino acids in translation. Proceedings of the National Academy of Sciences of the United States of America 106, 50-54 (2009).
23. F. Loayza-Puch et al., Tumour-specific proline vulnerability uncovered by differential ribosome codon reading. Nature 530, 490-494 (2016).
24. S. Schwörer et al., Proline biosynthesis is a vent for TGFbeta-induced mitochondrial redox stress. The EMBO journal, e103334 (2020).
25. I. V. Yang et al., Expression of cilium-associated genes defines novel molecular subtypes of idiopathic pulmonary fibrosis. Thorax 68, 1114-1121 (2013).
26. P. S. Ward et al., The potential for isocitrate dehydrogenase mutations to produce 2-hydroxyglutarate depends on allele specificity and subcellular compartmentalization. The Journal of biological chemistry 288, 3804-3815 (2013).
27. L. Salamanca-Cardona et al., In Vivo Imaging of Glutamine Metabolism to the Oncometabolite 2-Hydroxyglutarate in IDH1/2 Mutant Tumors. Cell metabolism 26, 830-841 e833 (2017).
28. S. Baghirova, B. G. Hughes, M. J. Hendzel, R. Schulz, Sequential fractionation and isolation of subcellular proteins from tissue or cultured cells. MethodsX 2, 440-445 (2015).
29. R. Hwang. T. Moore, T. Arumugam, V. Ramachandran, K. D. Amos, A. Rivera, B. Ji, D. B. Evans, C. D. Logsdon, Cancer-associated stromal fibroblasts promote pancreatic tumor progression, Cancer Res., 68, 918-926 (2008).
30. P. Millard, F. Letisse, S. Sokol, J.-C. Portais, IsoCor: correcting MS data in isotope labeling experiments, Bioinformatics, 28, 1294-1296 (2012).
31. The AACR Project GENIE Consortium, Cancer Discovery; 7(8): 818-831 (2017).
32. Intlekofer et al., Hypoxia Induces Production of L-2-Hydroxyglutrarate, Cell Metabolism, 304-311 (2015).

### EQUIVALENTS AND SCOPE

In the claims articles such as “a.” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of.” or “exactly one of.” “Consisting essentially of.” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising.” “including,” “carrying.” “having.” “containing.” “involving.” “holding.” “composed of.” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B.” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A method of treating a cancer characterized as having an isocitrate dehydrogenase 2 (IDH2) mutation, the method comprising:
administering to a subject in need thereof an antagonist of nicotinamide adenine dinucleotide kinase 2 (NADK2) in an amount effective to treat the cancer.

2. The method of claim 1, wherein the cancer is characterized as having increased levels of 2-hydroxyglutarate (2HG) relative to a known reference value.

3. The method of claim 1 or 2, wherein the cancer is characterized as having decreased levels of alpha-ketoglutarate (αKG) relative to a known reference value.

4. The method of claim 2 or 3, wherein the known reference value is from a cell characterized as not having the IDH2 mutation.

5. The method of claim 4, wherein the cell is a non-cancerous cell of the subject.

6. The method of any one of claims 1-5, wherein the IDH2 mutation produces a mutant IDH2 protein having a neomorphic enzymatic activity.

7. The method of claim 6, wherein the neomorphic enzymatic activity is a reduction of αKG to 2HG.

8. The method of any one of claims 1-7, wherein the IDH2 mutation is selected from R172S, exon 4 mutation, a codon 140 missense mutation, R140Q, a codon 172 missense mutation, R172K, an amplification of IDH2, a loss of IDH2, R172W, R172M, R140W, R172G, V305M, H384Q, T350P, R172T, V355I, K155N, A416V, W21S, X39 splice, R159H, A347T, D390Y, D259N, A370T, A174T, or a combination thereof.

9. The method of any one of claims 1-8, wherein the cancer is an adenocarcinoma.

10. The method of claim 9, wherein the adenocarcinoma is selected from colon adenocarcinoma, lung adenocarcinoma, high grade ovarian serous adenocarcinoma, colorectal adenocarcinoma, rectal adenocarcinoma, prostate adenocarcinoma, or a combination thereof.

11. The method of any one of claims 1-8, wherein the cancer is a carcinoma.

12. The method of claim 11, wherein the carcinoma is selected from breast invasive ductal carcinoma, intrahepatic cholangiocarcinoma, endometrial endometrioid carcinoma, bladder urothelial carcinoma, endometrial carcinoma, squamous cell lung carcinoma, or a combination thereof.

13. The method of any one of claims 1-8, wherein the cancer is selected from acute myeloid leukemia, oligodendroglioma, myelodysplastic syndrome, cutaneous melanoma, glioblastoma multiforme, angioimmunoblastic T-cell lymphoma, acute monoblastic and monocytic leukemia, or a combination thereof.

14. The method of any one of claims 1-13, wherein the cancer is characterized as not having an isocitrate dehydrogenase 1 (IDH1) mutation.

15. A method of treating a fibrotic disorder, the method comprising:
administering to a subject in need thereof an antagonist of nicotinamide adenine dinucleotide kinase 2 (NADK2) in an amount effective to treat the fibrotic disorder.

16. The method of claim 15, wherein the fibrotic disorder is characterized by increased levels of NADK2 relative to a known reference value.

17. The method of claim 15 or 16, wherein the fibrotic disorder is characterized by increased levels of pyrroline-5-carboxylate synthase (P5CS) relative to a known reference value.

18. The method of claim 16 or 17, wherein the known reference value is from a normal cell of the subject.

19. The method of any one of claims 15-18, wherein the fibrotic disorder is characterized by increased levels of an extracellular matrix protein.

20. The method of claim 19, wherein the extracellular matrix protein is collagen, elastin, fibronectin, and/or laminin.

21. The method of any one of claims 15-20, wherein the fibrotic disorder is pulmonary fibrosis or liver fibrosis.

22. A method for inhibiting cancer cell proliferation, the method comprising:
contacting cancer cells expressing a mutant isocitrate dehydrogenase 2 (IDH2) protein with an antagonist of nicotinamide adenine dinucleotide kinase 2 (NADK2), wherein the mutant IDH2 protein has a neomorphic enzymatic activity.

23. The method of claim 22, wherein the cancer cells contain increased levels of 2-hydroxyglutarate (2HG) relative to a known reference value.

24. The method of claim 22 or 23, wherein the cancer cells contain reduced levels of alpha-ketoglutarate (αKG) relative to a known reference value.

25. The method of claim 23 or 24, wherein the known reference value is from a non-cancerous cell and/or a cell that does not express the mutant IDH2 protein.

26. The method of any one of claims 22-25, wherein the neomorphic enzymatic activity is a reduction of αKG to 2HG.

27. The method of any one of claims 22-26, wherein the mutant IDH2 protein comprises one or more IDH2 mutations selected from R172S, exon 4 mutation, a codon 140 missense mutation, R140Q, a codon 172 missense mutation, R172K, an amplification of IDH2, a loss of IDH2, R172W, R172M, R140W, R172G, V305M, H384Q, T350P, R172T, V355I, K155N, A416V, W21S, X39 splice, R159H, A347T, D390Y, D259N, A370T, and A174T.

28. A method for inhibiting protein synthesis, the method comprising:
contacting a cell from a population of cells with an antagonist of nicotinamide adenine dinucleotide kinase 2 (NADK2).

29. The method of claim 28, wherein protein synthesis in the cell is decreased as compared to a cell that has not been contacted with the antagonist.

30. The method of claim 28 or 29, wherein the cell that has not been contacted with the antagonist is from the population of cells.

31. The method of any one of claims 28-30, wherein the cell from the population of cells is contacted with the antagonist in a nutrient-deficient environment.

32. The method of claim 31, wherein the nutrient-deficient environment has reduced levels of one or more amino acids compared to a nutrient-replete environment.

33. The method of claim 31 or 32, wherein the nutrient-deficient environment contains a maximum of 300 μM of proline.

34. The method of any one of claims 28-33, wherein the protein is collagen, elastin, fibronectin, and/or laminin.

35. The method of claim 34, wherein collagen synthesis is decreased in the cell contacted with the NADK2 antagonist as measured by staining collagen protein.

36. The method of claim 35, wherein collagen protein is stained by Picrosirius red staining.

37. The method of any one of claims 28-32, wherein proline biosynthesis is decreased in the cell contacted with the NADK2 antagonist as measured by gas chromatography-mass spectrometry (GC-MS) and/or liquid chromatography-mass spectrometry (LC-MS).

38. The method of claim 37, wherein proline is labeled with an isotopologue.

39. A method for inhibiting cell proliferation, the method comprising:
providing a population of cells in a nutrient-deficient environment; and
contacting a test cell portion of the population with an antagonist of nicotinamide adenine dinucleotide kinase 2 (NADK2), wherein the test cell portion has decreased proliferation compared to a control cell portion of the population.

40. The method of claim 39, wherein the control cell portion has not been contacted with the antagonist.

41. The method of claim 39 or 40, wherein the nutrient-deficient environment is deficient in one or more amino acids.

42. The method of claim 41, wherein the nutrient-deficient environment is deficient in proline.

43. The method of any one of claims 39-42, wherein cell proliferation is measured by cell number fold change compared to a cell not contacted with the antagonist.

44. A composition, comprising:
i) a nutrient-deficient cell culture medium; and
ii) an antagonist of nicotinamide adenine dinucleotide kinase 2 (NADK2).

45. The composition of claim 44, wherein the nutrient-deficient cell culture medium is deficient in one or more amino acids.

46. The composition of claim 44 or 45, further comprising:
iii) a population of cells.

47. The composition of claim 46, wherein the population of cells comprises cancer cells.

48. The composition of claim 47, wherein the cancer cells express a mutant isocitrate dehydrogenase 2 (IDH2) protein.

49. The composition of claim 48, wherein the mutant IDH2 protein has a neomorphic enzymatic activity.

50. The composition of claim 49, wherein the neomorphic enzymatic activity is a reduction of alpha-ketoglutarate (αKG) to 2-hydroxyglutarate (2HG).

51. The composition of any one of claims 48-50, wherein the mutant IDH2 protein comprises one or more IDH2 mutations selected from R172S, exon 4 mutation, a codon 140 missense mutation, R140Q, a codon 172 missense mutation, R172K, an amplification of IDH2, a loss of IDH2, R172W, R172M, R140W, R172G, V305M, H384Q, T350P, R172T, V355I, K155N, A416V, W21S, X39 splice, R159H, A347T, D390Y, D259N, A370T, and A174T.

52. The composition of any one of claims 47-51, wherein the cancer cells contain increased levels of 2HG relative to a known reference value.

53. The composition of any one of claims 47-52, wherein the cancer cells contain reduced levels of αKG relative to a known reference value.

54. The composition of claim 52 or 53, wherein the known reference value is from a non-cancerous cell and/or a cell that does not express a mutant IDH2 protein.

55. The composition of any one of claims 47-54, wherein the cancer is an adenocarcinoma.

56. The composition of claim 55, wherein the adenocarcinoma is selected from colon adenocarcinoma, lung adenocarcinoma, high grade ovarian serous adenocarcinoma, colorectal adenocarcinoma, rectal adenocarcinoma, prostate adenocarcinoma, or a combination thereof.

57. The composition of any one of claims 47-54, wherein the cancer is a carcinoma.

58. The composition of claim 57, wherein the carcinoma is selected from breast invasive ductal carcinoma, intrahepatic cholangiocarcinoma, endometrial endometrioid carcinoma, bladder urothelial carcinoma, endometrial carcinoma, squamous cell lung carcinoma, or a combination thereof.

59. The composition of any one of claims 47-54, wherein the cancer is selected from acute myeloid leukemia, oligodendroglioma, myelodysplastic syndrome, cutaneous melanoma, glioblastoma multiforme, angioimmunoblastic T-cell lymphoma, acute monoblastic and monocytic leukemia, or a combination thereof.

60. The composition of any one of claims 47-59, wherein the cancer is characterized as not having an isocitrate dehydrogenase 1 (IDH1) mutation.

61. The composition of any one of claims 44-60, wherein the nutrient-deficient cell culture medium comprises 10% serum, 100 units/mL penicillin, and/or 100 μg/mL streptomycin.

62. A method for decreasing protein synthesis, the method comprising:
providing a cell expressing nicotinamide adenine dinucleotide kinase 2 (NADK2) in a nutrient-deficient environment; and
contacting the cell with an antagonist of NADK2, wherein the cell contacted with the antagonist has decreased protein synthesis compared to a control cell not contacted with the antagonist.

63. The method of claim 62, wherein the protein is collagen, elastin, fibronectin, and/or laminin.

64. The method of claim 62 or 63, wherein the nutrient-deficient environment is deficient in one or more amino acids.

65. The method of any one of claims 62-64, wherein the nutrient-deficient environment is in vitro.

66. The method of any one of claims 62-64, wherein the nutrient-deficient environment is in vivo.

67. The method of any one of claims 62-66, wherein the cell contacted with the antagonist has reduced survival and/or proliferation compared to the control cell not contacted with the antagonist.

68. The method of any one of claims 62-67, wherein the cell contacted with the antagonist expresses pyrroline-5-carboxylate synthase (P5CS).

69. The method of any one of claims 62-68, wherein the cell contacted with the antagonist is associated with a fibrotic disorder.

70. The method of claim 69, wherein the fibrotic disorder is pulmonary fibrosis or liver fibrosis.

71. The method of claim 69 or 70, wherein the cell contacted with the antagonist expresses increased levels of NADK2 compared to a cell not associated with a fibrotic disorder.

72. The method of any one of claims 69-71, wherein the cell contacted with the antagonist expresses increased levels of P5CS compared to a cell not associated with a fibrotic disorder.

73. The method of any one of claims 66-72, wherein the nutrient-deficient environment comprises a subject on a restrictive diet.
